SEQUENCE LISTING (216 sequences)

SEQ ID NO: 1 - protein, 30 aa, Artificial Sequence
  Description of Artificial Sequence Synthetic polypeptide
  MISC_FEATURE (1)..(30): This sequence may encompass 1-6 “Gly Gly Gly Gly Ser” repeating units
GGGGSGGGGSGGGGSGGGGSGGGGSGGGGS

SEQ ID NO: 2 - protein, 4 aa, Artificial Sequence
  Description of Artificial Sequence Synthetic peptide
GSGG

SEQ ID NO: 3 - protein, 9 aa, Artificial Sequence
  Description of Artificial Sequence Synthetic peptide
GGGGSGGGS

SEQ ID NO: 4 - protein, 18 aa, Artificial Sequence
  Description of Artificial Sequence Synthetic peptide
KESGSVSSEQLAQFRSLD

SEQ ID NO: 5 - protein, 14 aa, Artificial Sequence
  Description of Artificial Sequence Synthetic peptide
EGKSSGSGSESKST

SEQ ID NO: 6 - protein, 6 aa, Artificial Sequence
  Description of Artificial Sequence Synthetic 6xHis tag
HHHHHH

SEQ ID NO: 7 - protein, 306 aa, Unknown
  Description of Unknown Protein sequence of 3VGR19-CD-H
MQVQLQESGGGSVQAGGSLRLSCVASGYSVSKNSMGWFRQAPGKEREGVAGINEGGTATYYANSVKGRFTISQDNAKNTVYLQMNTLKPEDTAIYYCAAGSPDPPPLIESVVKYWGQGTQVTVSSGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 8 - DNA, 921 bp, Unknown
  Description of Unknown Polynucleotide sequence encodes 3VGR19-CD-H
atgcaggttcagctgcaagaaagcggtggtggtagcgttcaggcaggcggtagcctgcgtctgagctgtgttgcaagcggttatagcgttagcaaaaattcaatgggttggtttcgtcaggcaccgggtaaagaacgtgaaggtgttgcaggtattaatgaaggtggcaccgcaacctattatgccaatagcgttaaaggtcgttttaccattagccaggataatgccaaaaataccgtttacctgcagatgaataccctgaaaccggaagataccgccatctattattgtgcagcaggtagtccggatccgcctccgctgattgaaagcgttgtgaaatattggggtcagggcacccaggttaccgttagctcaggtggtggtggcagcggtggcggtggttctgggggtggtggttcagtcacaggaggcatggcttcaaagtgggaccagaagggcatggacattgcctatgaggaggctgctctgggctacaaggagggaggggtcccaattggtggctgcctcatcaacaacaaggatggcagtgtcctgggcaggggccacaacatgaggttccagaagggcagtgccaccctgcatggggagatcagcaccctggagaactgtggcaggctggagggcaaggtctacaaggacaccactctgtacaccaccctcagcccttgtgacatgtgcacaggggccatcatcatgtatggcattcccaggtgtgtggtgggagagaatgtcaacttcaagtcaaaaggagagaagtacctccagaccaggggccatgaggtggttgtggtggatgatgagaggtgcaagaagattatgaagcagttcattgatgagagaccccaggactggtttgaggacattggggagggcagcagccatcatcatcatcatcactaa

SEQ ID NO: 9 - protein, 306 aa, Unknown
  Description of Unknown Protein sequence of 4VGR17-CD-H
MQVQLQESGGGSVQAGGSLRLSCVASGYSVSKNSMGWFRQAPGKEREGVAGINYGGTATYYANSVKGRFTISQDNAKNTVYLQMNTLKPEDTAIYYCAAGSPDPPPLIEGVVKYWGQGTQVTVSSGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 10 - DNA, 921 bp, Unknown
  Description of Unknown Polynucleotide sequence encodes 4VGR17-CD-H
atgcaggttcagctgcaagaaagcggtggtggtagcgttcaggcaggcggtagcctgcgtctgagctgtgttgcaagcggttatagcgttagcaaaaattcaatgggttggtttcgtcaggcaccgggtaaagaacgtgaaggtgttgcaggtattaattatggtggcaccgcaacctattatgccaatagcgttaaaggtcgttttaccattagccaggataatgccaaaaataccgtttacctgcagatgaataccctgaaaccggaagataccgccatctattattgtgcagcaggtagtccggatccgcctccgctgattgaaggtgtggtgaaatattggggtcagggcacccaggttaccgttagctcaggtggtggtggcagcggtggcggtggttctgggggtggtggttcagtcacaggaggcatggcttcaaagtgggaccagaagggcatggacattgcctatgaggaggctgctctgggctacaaggagggaggggtcccaattggtggctgcctcatcaacaacaaggatggcagtgtcctgggcaggggccacaacatgaggttccagaagggcagtgccaccctgcatggggagatcagcaccctggagaactgtggcaggctggagggcaaggtctacaaggacaccactctgtacaccaccctcagcccttgtgacatgtgcacaggggccatcatcatgtatggcattcccaggtgtgtggtgggagagaatgtcaacttcaagtcaaaaggagagaagtacctccagaccaggggccatgaggtggttgtggtggatgatgagaggtgcaagaagattatgaagcagttcattgatgagagaccccaggactggtttgaggacattggggagggcagcagccatcatcatcatcatcactaa

SEQ ID NO: 11 - protein, 306 aa, Unknown
  Description of Unknown Protein sequence of 4VGR38-CD-H
MQVQLQESGGGSVQAGGSLTLSCEVSGYSVNSNSMGWFRQAPGKEREGVAGINEGGVSTYYANFVQGRFTISQDNAKNMVYLQMNSLKPEDTAIYYCAAGAPDPPPLIESIVKYWGQGTQVTVSSGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 12 - DNA, 921 bp, Unknown
  Description of Unknown Polynucleotide sequence encodes 4VGR38-CD-H
atgcaggttcagctgcaagaaagcggtggtggtagcgttcaggcaggcggtagcctgaccctgagctgtgaagttagcggttatagcgttaatagcaatagcatgggttggtttcgtcaggcaccgggtaaagaacgtgaaggtgttgcaggtattaatgaaggtggtgttagcacctattatgccaattttgttcagggtcgttttaccattagccaggataatgcaaaaaacatggtttacctgcagatgaatagcctgaaaccggaagataccgcaatctattattgtgcagcgggtgcaccggatccgcctccgctgattgaaagcattgtgaaatattggggtcagggcacccaggttaccgttagctcaggtggtggtggcagcggtggcggtggttctgggggtggtggttcagtcacaggaggcatggcttcaaagtgggaccagaagggcatggacattgcctatgaggaggctgctctgggctacaaggagggaggggtcccaattggtggctgcctcatcaacaacaaggatggcagtgtcctgggcaggggccacaacatgaggttccagaagggcagtgccaccctgcatggggagatcagcaccctggagaactgtggcaggctggagggcaaggtctacaaggacaccactctgtacaccaccctcagcccttgtgacatgtgcacaggggccatcatcatgtatggcattcccaggtgtgtggtgggagagaatgtcaacttcaagtcaaaaggagagaagtacctccagaccaggggccatgaggtggttgtggtggatgatgagaggtgcaagaagattatgaagcagttcattgatgagagaccccaggactggtttgaggacattggggagggcagcagccatcatcatcatcatcactaa

SEQ ID NO: 13 - protein, 306 aa, Unknown
  Description of Unknown Protein sequence of VHH122-CD-H
MEVQLQESGGGLVQAGDSLRLSCLVSGRSFNSYTMGWFRQAPGKEREFVAAILWSGPTTYYADSVKGRFTISRDNAKNTVYLQMNSLKPEDTAVYYCAAALGVLVLAPGNVYSYWGQGTQVTVSSGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 14 - DNA, 921 bp, Unknown
  Description of Unknown Polynucleotide sequence encodes VHH122-CD-H
atggaagttcagctgcaagaaagcggtggtggtctggttcaggcaggcgatagcctgcgtctgagctgtctggttagcggtcgtagctttaatagctataccatgggttggtttcgtcaggcaccgggtaaagaacgtgaatttgttgcagcaattctgtggtcaggtccgaccacctattatgcagatagcgttaaaggtcgttttaccattagccgtgataatgccaaaaataccgtttacctgcagatgaatagcctgaaaccggaagataccgcagtgtattattgtgcagcagcactgggtgttctggtgctggctccgggtaatgtttatagttattggggtcagggcacccaggttaccgttagctcaggtggtggtggtagcggtggcggtggctctggtggtggcggttcagtcacaggaggcatggcttcaaagtgggaccagaagggcatggacattgcctatgaggaggctgctctgggctacaaggagggaggggtcccaattggtggctgcctcatcaacaacaaggatggcagtgtcctgggcaggggccacaacatgaggttccagaagggcagtgccaccctgcatggggagatcagcaccctggagaactgtggcaggctggagggcaaggtctacaaggacaccactctgtacaccaccctcagcccttgtgacatgtgcacaggggccatcatcatgtatggcattcccaggtgtgtggtgggagagaatgtcaacttcaagtcaaaaggagagaagtacctccagaccaggggccatgaggtggttgtggtggatgatgagaggtgcaagaagattatgaagcagttcattgatgagagaccccaggactggtttgaggacattggggagggcagcagccatcatcatcatcatcactaa

SEQ ID NO: 15 - protein, 306 aa, Unknown
  Description of Unknown Protein sequence of 7D12-CD-H
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 16 - DNA, 921 bp, Unknown
  Description of Unknown Polynucleotide sequence encodes 7D12-CD-H
atgcaggtgaaactggaagaaagcggtggtggtagcgttcagaccggtggtagcctgcgtctgacctgtgcagcaagcggtcgtaccagccgtagctatggtatgggttggtttcgtcaggcaccgggtaaagaacgtgaatttgttagcggtattagctggcgtggtgatagcaccggttatgcagatagcgttaaaggtcgttttaccattagccgtgataatgccaaaaataccgttgacctgcagatgaatagcctgaaaccggaagataccgcaatctattattgtgcagcagcagccggtagcgcctggtatggcaccctgtatgaatatgattattggggtcagggcacccaggttaccgttagctcaggtggtggtggcagcggtggcggtggttcaggtggcggaggtagcgtcacaggaggcatggcttcaaagtgggaccagaagggcatggacattgcctatgaggaggctgctctgggctacaaggagggaggggtcccaattggtggctgcctcatcaacaacaaggatggcagtgtcctgggcaggggccacaacatgaggttccagaagggcagtgccaccctgcatggggagatcagcaccctggagaactgtggcaggctggagggcaaggtctacaaggacaccactctgtacaccaccctcagcccttgtgacatgtgcacaggggccatcatcatgtatggcattcccaggtgtgtggtgggagagaatgtcaacttcaagtcaaaaggagagaagtacctccagaccaggggccatgaggtggttgtggtggatgatgagaggtgcaagaagattatgaagcagttcattgatgagagaccccaggactggtttgaggacattggggagggcagcagccatcatcatcatcatcactaa

SEQ ID NO: 17 - protein, 306 aa, Unknown
  Description of Unknown Protein sequence of 7D12-CDoem3-H
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 18 - DNA, 921 bp, Unknown
  Description of Unknown Polynucleotide sequence encodes 7D12-CDoem3-H
atgcaggtgaaactggaagaaagcggtggtggtagcgttcagaccggtggtagcctgcgtctgacctgtgcagcaagcggtcgtaccagccgtagctatggtatgggttggtttcgtcaggcaccgggtaaagaacgtgaatttgttagcggtattagctggcgtggtgatagcaccggttatgcagatagcgttaaaggtcgttttaccattagccgtgataatgccaaaaataccgttgacctgcagatgaatagcctgaaaccggaagataccgcaatctattattgtgcagcagcagccggtagcgcctggtatggcaccctgtatgaatatgattattggggtcagggcacccaggttaccgttagctcaggtggtggtggcagcggtggcggtggttcaggtggcggaggtagcgttaccggtggtatggcaagcaaatgggatcagaaaggtatggatattgcatatgaagaagcactgctgggttataaagaaggtggcgttccgattggtggttgtctgattaataacaaagatggtagcgttctgggtcgcggtcataatatgcgttttcagaaaggcagcgcaaccctgcatggtgaaattagcaccctggaaaattgtggtcgtctggaaggtaaagtgtataaagataccaccctgtataccacactgagcccgtgtgatatgtgtaccggtgccattattatgtatggtattccgcgttgtgtgattggcgaaaacgttaactttaaaagcaaaggcgagaaatatctgcagacccgtggtcatgaagttgttgttgtggatgatgaacgttgcaaaaaactgatgaaacagtttatcgatgaacgtccgcaggattggtttgaagatattggtgaaggcagcagccatcatcatcatcatcactaa

SEQ ID NO: 19 - protein, 297 aa, Unknown
  Description of Unknown Protein sequence of 7D12-CDoem3
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGE

SEQ ID NO: 20 - DNA, 894 bp, Unknown
  Description of Unknown Polynucleotide sequence encodes 7D12-CDoem3
atgcaggtgaaactggaagaaagcggtggtggtagcgttcagaccggtggtagcctgcgtctgacctgtgcagcaagcggtcgtaccagccgtagctatggtatgggttggtttcgtcaggcaccgggtaaagaacgtgaatttgttagcggtattagctggcgtggtgatagcaccggttatgcagatagcgttaaaggtcgttttaccattagccgtgataatgccaaaaataccgttgacctgcagatgaatagcctgaaaccggaagataccgcaatctattattgtgcagcagcagccggtagcgcctggtatggcaccctgtatgaatatgattattggggtcagggcacccaggttaccgttagctcaggtggtggtggcagcggtggcggtggttcaggtggcggaggtagcgttaccggtggtatggcaagcaaatgggatcagaaaggtatggatattgcatatgaagaagcactgctgggttataaagaaggtggcgttccgattggtggttgtctgattaataacaaagatggtagcgttctgggtcgcggtcataatatgcgttttcagaaaggcagcgcaaccctgcatggtgaaattagcaccctggaaaattgtggtcgtctggaaggtaaagtgtataaagataccaccctgtataccacactgagcccgtgtgatatgtgtaccggtgccattattatgtatggtattccgcgttgtgtgattggcgaaaacgttaactttaaaagcaaaggcgagaaatatctgcagacccgtggtcatgaagttgttgttgtggatgatgaacgttgcaaaaaactgatgaaacagtttatcgatgaacgtccgcaggattggtttgaagatattggtgaataa

SEQ ID NO: 21 - protein, 157 aa, Unknown
  Description of Unknown Protein sequence of yeast cytosine deaminase
VTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 22 - protein, 157 aa, Unknown
  Description of Unknown Protein sequence of mutant yeast cytosine deaminase
VTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGE

SEQ ID NO: 23 - protein, 124 aa, Unknown
  Description of Unknown Protein sequence of 3VGR19
QVQLQESGGGSVQAGGSLRLSCVASGYSVSKNSMGWFRQAPGKEREGVAGINEGGTATYYANSVKGRFTISQDNAKNTVYLQMNTLKPEDTAIYYCAAGSPDPPPLIESVVKYWGQGTQVTVSS

SEQ ID NO: 24 - protein, 124 aa, Unknown
  Description of Unknown Protein sequence of 4VGR17
QVQLQESGGGSVQAGGSLRLSCVASGYSVSKNSMGWFRQAPGKEREGVAGINYGGTATYYANSVKGRFTISQDNAKNTVYLQMNTLKPEDTAIYYCAAGSPDPPPLIEGVVKYWGQGTQVTVSS

SEQ ID NO: 25 - protein, 124 aa, Unknown
  Description of Unknown Protein sequence of 4VGR38
QVQLQESGGGSVQAGGSLTLSCEVSGYSVNSNSMGWFRQAPGKEREGVAGINEGGVSTYYANFVQGRFTISQDNAKNMVYLQMNSLKPEDTAIYYCAAGAPDPPPLIESIVKYWGQGTQVTVSS

SEQ ID NO: 26 - protein, 124 aa, Unknown
  Description of Unknown Protein sequence of VHH122
EVQLQESGGGLVQAGDSLRLSCLVSGRSFNSYTMGWFRQAPGKEREFVAAILWSGPTTYYADSVKGRFTISRDNAKNTVYLQMNSLKPEDTAVYYCAAALGVLVLAPGNVYSYWGQGTQVTVSS

SEQ ID NO: 27 - protein, 124 aa, Unknown
  Description of Unknown Protein sequence of 7D12
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 28 - protein, 5 aa, Unknown
  Description of Unknown 3VGR19 CDR1 sequence
KNSMG

SEQ ID NO: 29 - protein, 17 aa, Unknown
  Description of Unknown 3VGR19 CDR2 sequence
GINEGGTATYYANSVKG

SEQ ID NO: 30 - protein, 15 aa, Unknown
  Description of Unknown 3VGR19 CDR3 sequence
GSPDPPPLIESVVKY

SEQ ID NO: 31 - protein, 5 aa, Unknown
  Description of Unknown 4VGR17 CDR1 sequence
KNSMG

SEQ ID NO: 32 - protein, 17 aa, Unknown
  Description of Unknown 4VGR17 CDR2 sequence
GINYGGTATYYANSVKG

SEQ ID NO: 33 - protein, 15 aa, Unknown
  Description of Unknown 4VGR17 CDR3 sequence
GSPDPPPLIEGVVKY

SEQ ID NO: 34 - protein, 5 aa, Unknown
  Description of Unknown 4VGR38 CDR1 sequence
SNSMG

SEQ ID NO: 35 - protein, 17 aa, Unknown
  Description of Unknown 4VGR38 CDR2 sequence
GINEGGVSTYYANFVQG

SEQ ID NO: 36 - protein, 15 aa, Unknown
  Description of Unknown 4VGR38 CDR3 sequence
GAPDPPPLIESIVKY

SEQ ID NO: 37 - protein, 5 aa, Unknown
  Description of Unknown VHH122 CDR1 sequence
SYTMG

SEQ ID NO: 38 - protein, 17 aa, Unknown
  Description of Unknown VHH122 CDR2 sequence
AILWSGPTTYYADSVKG

SEQ ID NO: 39 - protein, 15 aa, Unknown
  Description of Unknown VHH122 CDR3 sequence
ALGVLVLAPGNVYSY

SEQ ID NO: 40 - protein, 5 aa, Unknown
  Description of Unknown 7D12 CDR1 sequence
SYGMG

SEQ ID NO: 41 - protein, 17 aa, Unknown
  Description of Unknown 7D12 CDR2 sequence
GISWRGDSTGYADSVKG

SEQ ID NO: 42 - protein, 15 aa, Unknown
  Description of Unknown 7D12 CDR3 sequence
AAGSAWYGTLYEYDY

SEQ ID NO: 43 - protein, 30 aa, Unknown
  Description of Unknown 3VGR19 FR1 sequence
QVQLQESGGGSVQAGGSLRLSCVASGYSVS

SEQ ID NO: 44 - protein, 14 aa, Unknown
  Description of Unknown 3VGR19 FR2 sequence
WFRQAPGKEREGVA

SEQ ID NO: 45 - protein, 32 aa, Unknown
  Description of Unknown 3VGR19 FR3 sequence
RFTISQDNAKNTVYLQMNTLKPEDTAIYYCAA

SEQ ID NO: 46 - protein, 11 aa, Unknown
  Description of Unknown 3VGR19 FR4 sequence
WGQGTQVTVSS

SEQ ID NO: 47 - protein, 30 aa, Unknown; identical to SEQ ID NO: 43
  Description of Unknown 4VGR17 FR1 sequence

SEQ ID NO: 48 - protein, 14 aa, Unknown
  Description of Unknown 4VGR17 FR2 sequence
WFRQAPGKEREGVA

SEQ ID NO: 49 - protein, 32 aa, Unknown; identical to SEQ ID NO: 45
  Description of Unknown 4VGR17 FR3 sequence

SEQ ID NO: 50 - protein, 11 aa, Unknown
  Description of Unknown 4VGR17 FR4 sequence
WGQGTQVTVSS

SEQ ID NO: 51 - protein, 30 aa, Unknown
  Description of Unknown 4VGR38 FR1 sequence
QVQLQESGGGSVQAGGSLTLSCEVSGYSVN

SEQ ID NO: 52 - protein, 14 aa, Unknown
  Description of Unknown 4VGR38 FR2 sequence
WFRQAPGKEREGVA

SEQ ID NO: 53 - protein, 32 aa, Unknown
  Description of Unknown 4VGR38 FR3 sequence
RFTISQDNAKNMVYLQMNSLKPEDTAIYYCAA

SEQ ID NO: 54 - protein, 11 aa, Unknown
  Description of Unknown 4VGR38 FR4 sequence
WGQGTQVTVSS

SEQ ID NO: 55 - protein, 30 aa, Unknown
  Description of Unknown VHH122 FR1 sequence
EVQLQESGGGLVQAGDSLRLSCLVSGRSFN

SEQ ID NO: 56 - protein, 14 aa, Unknown
  Description of Unknown VHH122 FR2 sequence
WFRQAPGKEREFVA

SEQ ID NO: 57 - protein, 32 aa, Unknown
  Description of Unknown VHH122 FR3 sequence
RFTISRDNAKNTVYLQMNSLKPEDTAVYYCAA

SEQ ID NO: 58 - protein, 11 aa, Unknown
  Description of Unknown VHH122 FR4 sequence
WGQGTQVTVSS

SEQ ID NO: 59 - protein, 30 aa, Unknown
  Description of Unknown 7D12 FR1 sequence
QVKLEESGGGSVQTGGSLRLTCAASGRTSR

SEQ ID NO: 60 - protein, 14 aa, Unknown
  Description of Unknown 7D12 FR2 sequence
WFRQAPGKEREFVS

SEQ ID NO: 61 - protein, 32 aa, Unknown
  Description of Unknown 7D12 FR3 sequence
RFTISRDNAKNTVDLQMNSLKPEDTAIYYCAA

SEQ ID NO: 62 - protein, 11 aa, Unknown
  Description of Unknown 7D12 FR4 sequence
WGQGTQVTVSS

SEQ ID NO: 63 - protein, 10 aa, Unknown
  Description of Unknown T cell epitope 1 sequence
SVQTGGSLRL

SEQ ID NO: 64 - protein, 9 aa, Unknown
  Description of Unknown T cell epitope 2 sequence
LKPEDTAIY

SEQ ID NO: 65 - protein, 10 aa, Unknown
  Description of Unknown T cell epitope 3 sequence
IYYCAAAAGS

SEQ ID NO: 66 - protein, 9 aa, Unknown
  Description of Unknown T cell epitope 4 sequence
YTTLSPCDM

SEQ ID NO: 67 - protein, 9 aa, Unknown
  Description of Unknown T cell epitope 5 sequence
MCTGAIIMY

SEQ ID NO: 68 - protein, 11 aa, Unknown
  Description of Unknown T cell epitope 6 sequence
VVVVDDERCKK

SEQ ID NO: 69 - protein, 124 aa, Unknown
  Description of Unknown Protein sequence of 2D3
EVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 70 - protein, 118 aa, Unknown
  Description of Unknown Protein sequence of 5F7
EVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSS

SEQ ID NO: 71 - protein, 119 aa, Unknown
  Description of Unknown Protein sequence of 47D5
KVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSS

SEQ ID NO: 72 - protein, 306 aa, Unknown
  Description of Unknown 2D3-CDoem3-H sequence
MEVQLVESGGSLVQPGGSLRLSCAASGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 73 - protein, 300 aa, Unknown
  Description of Unknown 5F7-CDoem3-H sequence
MEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 74 - protein, 301 aa, Unknown
  Description of Unknown 47D5-CDem3-H sequence
MKVQLVESGGGLVQPGGSLRLSCAASGSIFGFNDMAWYRQAPGKQRELVALISRVGVTSSADSVKGRFTISRVNAKDTVYLQMNSLKPEDTAVYYCYMDQRLDGSTLAYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 75 - protein, 127 aa, Unknown
  Description of Unknown BCD090-M2 sequence
QVQLVQSGGGLVQAGGSLRLSCAFSGRTFSMYTMGWFRQAPGKEREFVAANRGRGLSPDIADSVNGRFTISRDNAKNTLYLQMDSLKPEDTAVYYCAADLQYGSSWPQRSSAEYDYWGQGTTVTVSS

SEQ ID NO: 76 - protein, 309 aa, Unknown
  Description of Unknown BCD090-M2-CDoem3-H sequence
MQVQLVQSGGGLVQAGGSLRLSCAFSGRTFSMYTMGWFRQAPGKEREFVAANRGRGLSPDIADSVNGRFTISRDNAKNTLYLQMDSLKPEDTAVYYCAADLQYGSSWPQRSSAEYDYWGQGTTVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 77 - protein, 120 aa, Unknown
  Description of Unknown Anti-CEA vhh (ABS29544.1) sequence
EVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSS

SEQ ID NO: 78 - protein, 302 aa, Unknown
  Description of Unknown Anti-CEA (ABS29544.1)-CDoem3-H sequence
MEVQLVESGGGFVQAGESLTLSCTSSTLTFTPYRMAWYRQAPGKQRDLVADISSGDGRTTNYADFAKGRFTISRDNIKNTVFLRMTNLKPEDTAVYYCNTFVSFVGIARSWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 79 - protein, 123 aa, Unknown [sequence omitted]

SEQ ID NO: 80 - protein, 305 aa, Unknown
  Description of Unknown NbCEA5-CDoem3-H sequence
MQVQLVESGGGSVQAGGSLRLSCAASGDTYGSYWMGWFRQAPGKEREGVAAINRGGGYTVYADSVKGRFTISRDTAKNTVYLQMNSLRPDDTADYYCAASGVLGGLHEDWFNYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 81 - protein, 194 aa, Unknown
  Description of Unknown GE10-CD-H sequence
MLHLWVPEPWTQTGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 82 - protein, 194 aa, Unknown
  Description of Unknown GE11-CD-H sequence
MYHWYGYTPQNVIGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 83 - protein, 199 aa, Unknown
  Description of Unknown Inherbin1-CD-H sequence
MMLYNPTTYQMDVNPEGKGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 84 - protein, 199 aa, Unknown
  Description of Unknown Inherbin3-CD-H sequence
MLVYNKLTFQLEPNPHTKGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 85 - protein, 357 aa, Unknown
  Description of Unknown Endo-CD sequence
MHSHRDFQPVLHLVALNSPLSGGMRGIRGADFQCFQQARAVGLAGTFRAFLSSRLQDLYSIVRRADRAAVPIVNLKDELLFPSWEALFSGSEGPLKPGARIFSFDGKDVLRHPTWPQKSVWHGSDPNGRRLTESYCETWRTEAPSATGQASSLLGGRLLGQSAASCHHAYIVLCIENSFMTASKSGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 86 - protein, 306 aa, Unknown
  Description of Unknown 3VGR10-CD-H sequence
MQVQLQESGGGSVQAGGSLRLSCVASGYSVSKNSMGWFRQAPGKEREGVAGINEGGTATYYANSVKGRFTISQDNARNTVYLQMNTLKPEDTAIYYCAAGSPDPPPLIESVVKYWGQGTQVTVSSGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 87 - protein, 304 aa, Unknown
  Description of Unknown 3VGR17-CD-H sequence
MQVQLQESGGGSVQAGGSLRLSCAASGYTYCSYDMGWYRQAPGKEREFVSAIDNYGRTGYADSVKGRFTISQDNAKNTSYLQMNSLKPEDTAIYYCKVDVCPDPSRPGRPGYWGQGTQVTVSSGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 88 - protein, 290 aa, Unknown
  Description of Unknown DOM-16-39-CD-H sequence
MDIQMTQSPTSLSASVGDRVTITCRASQWIGNLLDWYQQKPGEAPKLLIYYASFLQSGVPSRFSGGGFGTDFTLTISSLQPEDFATYYCQQANPAPLTFGQGTKVEIKRGGGGSGGGGSGGGGSVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSSHHHHHH

SEQ ID NO: 89 - protein, 802 aa, Unknown
  Description of Unknown SP-RituxanHC-CD-L-CD sequence
MKKNIAFLLASMFVFSIATNAYAQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGEGSTSGSGKSSEGKGVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 90 - protein, 236 aa, Unknown
  Description of Unknown SP-RituxanLC sequence
MKKNIAFLLASMFVFSIATNAYAQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 630 aa, Unknown
  Description of Unknown SP-HerceptinHC-CD sequence
MKKNIAFLLASMFVFSIATNAYAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 92 - protein, 237 aa, Unknown
  Description of Unknown SP-HerceptinLC sequence
MKKNIAFLLASMFVFSIATNAYADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 306 aa, Unknown
  Description of Unknown TC3-001 S12V sequence
MQVKLEESGGGVVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 94 - protein, 306 aa, Unknown
  Description of Unknown TC3-002 V13A sequence
MQVKLEESGGGSAQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 95 - protein, 306 aa, Unknown
  Description of Unknown TC3-003 T15V sequence
MQVKLEESGGGSVQVGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 96 - protein, 306 aa, Unknown
  Description of Unknown TC3-004 G16D sequence
MQVKLEESGGGSVQTDGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 97 - protein, 306 aa, Unknown
  Description of Unknown TC3-005 S18D sequence
MQVKLEESGGGSVQTGGDLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 98 - protein, 306 aa, Unknown
  Description of Unknown TC3-006 S18E sequence
MQVKLEESGGGSVQTGGELRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 99 - protein, 306 aa, Unknown
  Description of Unknown TC3-007 K88R sequence
MQVKLEESGGGSVQTGGSLRLTCAASGRTSRSYGMGWFRQAPGKEREFVSGISWRGDSTGYADSVKGRFTISRDNAKNTVDLQMNSLRPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 100 - protein, 306 aa, Unknown
  Description of Unknown TC3-008 P89A sequence
MQVKLEESGGGSVQTGGSLRLTCAASGRTSRSYGMGWFRQAPGKEREFVSGISWRGDSTGYADSVKGRFTISRDNAKNTVDLQMNSLKAEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 101 - protein, 306 aa, Unknown
  Description of Unknown TC3-009 K88A sequence
MQVKLEESGGGSVQTGGSLRLTCAASGRTSRSYGMGWFRQAPGKEREFVSGISWRGDSTGYADSVKGRFTISRDNAKNTVDLQMNSLAPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 102 - protein, 306 aa, Unknown
  Description of Unknown TC3-010 K88D sequence
MQVKLEESGGGSVQTGGSLRLTCAASGRTSRSYGMGWFRQAPGKEREFVSGISWRGDSTGYADSVKGRFTISRDNAKNTVDLQMNSLDPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 103 - protein, 306 aa, Unknown
  Description of Unknown TC3-011 K88S sequence
MQVKLEESGGGSVQTGGSLRLTCAASGRTSRSYGMGWFRQAPGKEREFVSGISWRGDSTGYADSVKGRFTISRDNAKNTVDLQMNSLSPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 104 - protein, 306 aa, Unknown
  Description of Unknown TC3-012 K88T sequence
MQVKLEESGGGSVQTGGSLRLTCAASGRTSRSYGMGWFRQAPGKEREFVSGISWRGDSTGYADSVKGRFTISRDNAKNTVDLQMNSLTPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 105 - protein, 306 aa, Unknown
  Description of Unknown TC3-013 P89D sequence
MQVKLEESGGGSVQTGGSLRLTCAASGRTSRSYGMGWFRQAPGKEREFVSGISWRGDSTGYADSVKGRFTISRDNAKNTVDLQMNSLKDEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 106 - protein, 306 aa, Unknown
  Description of Unknown TC3-014 P89E sequence
MQVKLEESGGGSVQTGGSLRLTCAASGRTSRSYGMGWFRQAPGKEREFVSGISWRGDSTGYADSVKGRFTISRDNAKNTVDLQMNSLKEEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 107 - protein, 306 aa, Unknown
  Description of Unknown TC3-015 T92D sequence
MQVKLEESGGGSVQTGGSLRLTCAASGRTSRSYGMGWFRQAPGKEREFVSGISWRGDSTGYADSVKGRFTISRDNAKNTVDLQMNSLKPEDDAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 108 - protein, 306 aa, Unknown
  Description of Unknown TC3-016 T92E sequence
MQVKLEESGGGSVQTGGSLRLTCAASGRTSRSYGMGWFRQAPGKEREFVSGISWRGDSTGYADSVKGRFTISRDNAKNTVDLQMNSLKPEDEAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 109 - protein, 306 aa, Unknown
  Description of Unknown TC3-017 T92G sequence
MQVKLEESGGGSVQTGGSLRLTCAASGRTSRSYGMGWFRQAPGKEREFVSGISWRGDSTGYADSVKGRFTISRDNAKNTVDLQMNSLKPEDGAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 110 - protein, 306 aa, Unknown
  Description of Unknown TC3-018 T92H sequence
MQVKLEESGGGSVQTGGSLRLTCAASGRTSRSYGMGWFRQAPGKEREFVSGISWRGDSTGYADSVKGRFTISRDNAKNTVDLQMNSLKPEDHAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 111 - protein, 306 aa, Unknown
  Description of Unknown TC3-019 T92Q sequence
MQVKLEESGGGSVQTGGSLRLTCAASGRTSRSYGMGWFRQAPGKEREFVSGISWRGDSTGYADSVKGRFTISRDNAKNTVDLQMNSLKPEDQAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 112 - protein, 306 aa, Unknown
  Description of Unknown TC3-020 A93D sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTDIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 113 - protein, 306 aa, Unknown
  Description of Unknown TC3-022 I94V sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAVYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 114 - protein, 306 aa, Unknown
  Description of Unknown TC3-023 I94A sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAAYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 115 - protein, 306 aa, Unknown
  Description of Unknown TC3-024 I94T sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTATYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 116 - protein, 306 aa, Unknown
  Description of Unknown TC3-025 I94R sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 117 - protein, 306 aa, Unknown
  Description of Unknown TC3-026 I94Q sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 118 - protein, 306 aa, Unknown
  Description of Unknown TC3-027 I94N sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTANYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 119 - protein, 306 aa, Unknown
  Description of Unknown TC3-028 Y224A sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLATTLSPCDMCTGAIIMYGIPRCVIGENVNFKSKGEKYLQTRGHEVVVVDDERCKKLMKQFIDERPQDWFEDIGEGSSHHHHHH

SEQ ID NO: 120 - protein, 306 aa, Unknown
  Description of Unknown TC3-029 Y224H sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLHTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 121 - protein, 306 aa, Unknown
  Description of Unknown TC3-030 T225D sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYDTLSPCDMCTGAIIMYGIPRCVIGENVNFKSKGEKYLQTRGHEVVVVDDERCKKLMKQFIDERPQDWFEDIGEGSSHHHHHH

SEQ ID NO: 122 - protein, 306 aa, Unknown
  Description of Unknown TC3-031 T226E sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTELSPCDMCTGAIIMYGIPRCVIGENVNFKSKGEKYLQTRGHEVVVVDDERCKKLMKQFIDERPQDWFEDIGEGSSHHHHHH

SEQ ID NO: 123 - protein, 306 aa, Unknown
  Description of Unknown TC3-032 M232N sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDNCTGAIIMYGIPRCVIGENVNFKSKGEKYLQTRGHEVVVVDDERCKKLMKQFIDERPQDWFEDIGEGSSHHHHHH

SEQ ID NO: 124 - protein, 306 aa, Unknown
  Description of Unknown TC3-033 M232A sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDACTGAIIMYGIPRCVIGENVNFKSKGEKYLQTRGHEVVVVDDERCKKLMKQFIDERPQDWFEDIGEGSSHHHHHH

SEQ ID NO: 125 - protein, 306 aa, Unknown
  Description of Unknown TC3-034 M232K sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDKCTGAIIMYGIPRCVIGENVNFKSKGEKYLQTRGHEVVVVDDERCKKLMKQFIDERPQDWFEDIGEGSSHHHHHH

SEQ ID NO: 126 - protein, 306 aa, Unknown
  Description of Unknown TC3-035 M232Q sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDQCTGAIIMYGIPRCVIGENVNFKSKGEKYLQTRGHEVVVVDDERCKKLMKQFIDERPQDWFEDIGEGSSHHHHHH

SEQ ID NO: 127 - protein, 306 aa, Unknown
  Description of Unknown TC3-036 V268A sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 128 - protein, 306 aa, Unknown
  Description of Unknown TC3-037 V268T sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 129 - protein, 306 aa, Unknown
  Description of Unknown TC3-038 V269A sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVAVVDDERCKKLMKQFIDERPQDWFEDIGEGSSHHHHHH

SEQ ID NO: 130 - protein, 306 aa, Unknown
  Description of Unknown TC3-039 V269L sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVLVVDDERCKKLMKQFIDERPQDWFEDIGEGSSHHHHHH

SEQ ID NO: 131 - protein, 306 aa, Unknown
  Description of Unknown TC3-040 V269I sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVIVVDDERCKKLMKQFIDERPQDWFEDIGEGSSHHHHHH

SEQ ID NO: 132 - protein, 306 aa, Unknown
  Description of Unknown TC3-041 V269T sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 133 - protein, 306 aa, Unknown
  Description of Unknown TC3-042 V270A sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVAVDDERCKKLMKQFIDERPQDWFEDIGEGSSHHHHHH

SEQ ID NO: 134 - protein, 306 aa, Unknown
  Description of Unknown TC3-043 V270T sequence
MQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVTVDDERCKKLMKQFIDERPQDWFEDIGEGSSHHHHHH

SEQ ID NO: 135 - protein, 306 aa, Unknown
  Description of Unknown TC3-044 S12V, T15V, K88R, P89A, I94V, V268A sequence
MQVKLEESGGGVVQVGGSLRLTCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 136 - protein, 306 aa, Unknown
  Description of Unknown TC3-045 S12V, T15V, K88R, P89A, I94V, V268T sequence
MQVKLEESGGGVVQVGGSLRLTCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 137 - protein, 306 aa, Unknown
  Description of Unknown TC3-046 S12V, T15V, K88R, P89A, I94V, V269T sequence
MQVKLEESGGGVVQVGGSLRLTCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 138 - protein, 306 aa, Unknown
  Description of Unknown TC3-047 V13A, K88D, I94R, V268A sequence
MQVKLEESGGGSAQTGGSLRLTCAASGRTSRSYGMGWFRQAPGKEREFVSGISWRGDSTGYADSVKGRFTISRDNAKNTVDLQMNSLDPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 139 - protein, 306 aa, Unknown
  Description of Unknown TC3-048 G16D, K88D, I94R, V268A sequence
MQVKLEESGGGSVQTDGSLRLTCAASGRTSRSYGMGWFRQAPGKEREFVSGISWRGDSTGYADSVKGRFTISRDNAKNTVDLQMNSLDPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 140 - protein, 306 aa, Unknown
  Description of Unknown TC3-049 S18D, K88D, I94R, V268A sequence
MQVKLEESGGGSVQTGGDLRLTCAASGRTSRSYGMGWFRQAPGKEREFVSGISWRGDSTGYADSVKGRFTISRDNAKNTVDLQMNSLDPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 141 - protein, 306 aa, Unknown
  Description of Unknown TC3-050 V13A, K88T, I94R, V268A sequence
MQVKLEESGGGSAQTGGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 142 - protein, 306 aa, Unknown
  Description of Unknown TC3-051 G16D, K88T, I94R, V268A sequence
MQVKLEESGGGSVQTDGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 143 - protein, 306 aa, Unknown
  Description of Unknown TC3-052 S18D, K88T, I94R, V268A sequence
MQVKLEESGGGSVQTGGDLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 144 - protein, 306 aa, Unknown
  Description of Unknown TC3-053 V13A, K88D, I94Q, V268A sequence
MQVKLEESGGGSAQTGGSLRLTCAASGRTSRSYGMGWFRQAPGKEREFVSGISWRGDSTGYADSVKGRFTISRDNAKNTVDLQMNSLDPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 145 - protein, 306 aa, Unknown
  Description of Unknown TC3-054 G16D, K88D, I94Q, V268A sequence
MQVKLEESGGGSVQTDGSLRLTCAASGRTSRSYGMGWFRQAPGKEREFVSGISWRGDSTGYADSVKGRFTISRDNAKNTVDLQMNSLDPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 146 - protein, 306 aa, Unknown
  Description of Unknown TC3-055 S18D, K88D, I94Q, V268A sequence
MQVKLEESGGGSVQTGGDLRLTCAASGRTSRSYGMGWFRQAPGKEREFVSGISWRGDSTGYADSVKGRFTISRDNAKNTVDLQMNSLDPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 147 - protein, 306 aa, Unknown
  Description of Unknown TC3-056 V13A, K88T, I94Q, V268A sequence
MQVKLEESGGGSAQTGGSLRLTCAASGRTSRSYGMGWFRQAPGKEREFVSGISWRGDSTGYADSVKGRFTISRDNAKNTVDLQMNSLTPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 148 - protein, 306 aa, Unknown
  Description of Unknown TC3-057 G16D, K88T, I94Q, V268A sequence
MQVKLEESGGGSVQTDGSLRLTCAASGRTSRSYGMGWFRQAPGKEREFVSGISWRGDSTGYADSVKGRFTISRDNAKNTVDLQMNSLTPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 149 - protein, 306 aa, Unknown
  Description of Unknown TC3-058 S18D, K88T, I94Q, V268A sequence
MQVKLEESGGGSVQTGGDLRLTCAASGRTSRSYGMGWFRQAPGKEREFVSGISWRGDSTGYADSVKGRFTISRDNAKNTVDLQMNSLTPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 150 - protein, 306 aa, Unknown
  Description of Unknown TC3-059 V13A, K88D, I94R, V268T sequence
MQVKLEESGGGSAQTGGSLRLTCAASGRTSRSYGMGWFRQAPGKEREFVSGISWRGDSTGYADSVKGRFTISRDNAKNTVDLQMNSLDPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 151 - protein, 306 aa, Unknown
  Description of Unknown TC3-060 G16D, K88D, I94R, V268T sequence
MQVKLEESGGGSVQTDGSLRLTCAASGRTSRSYGMGWFRQAPGKEREFVSGISWRGDSTGYADSVKGRFTISRDNAKNTVDLQMNSLDPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 152 - protein, 306 aa, Unknown
  Description of Unknown TC3-061 S18D, K88D, I94R, V268T sequence
MQVKLEESGGGSVQTGGDLRLTCAASGRTSRSYGMGWFRQAPGKEREFVSGISWRGDSTGYADSVKGRFTISRDNAKNTVDLQMNSLDPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 153 - protein, 306 aa, Unknown
  Description of Unknown TC3-062 V13A, K88T, I94R, V268T sequence
MQVKLEESGGGSAQTGGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 154 - protein, 306 aa, Unknown
  Description of Unknown TC3-063 G16D, K88T, I94R, V268T sequence
MQVKLEESGGGSVQTDGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 155 - protein, 306 aa, Unknown
  Description of Unknown TC3-064 S18D, K88T, I94R, V268T sequence
MQVKLEESGGGSVQTGGDLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 156 - protein, 306 aa, Unknown
  Description of Unknown TC3-065 V13A, K88D, I94Q, V268T sequence
MQVKLEESGGGSAQTGGSLRLTCAASGRTSRSYGMGWFRQAPGKEREFVSGISWRGDSTGYADSVKGRFTISRDNAKNTVDLQMNSLDPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 157 - protein, 306 aa, Unknown
  Description of Unknown TC3-066 G16D, K88D, I94Q, V268T sequence
MQVKLEESGGGSVQTDGSLRLTCAASGRTSRSYGMGWFRQAPGKEREFVSGISWRGDSTGYADSVKGRFTISRDNAKNTVDLQMNSLDPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 158 - protein, 306 aa, Unknown
  Description of Unknown TC3-067 S18D, K88D, I94Q, V268T sequence
MQVKLEESGGGSVQTGGDLRLTCAASGRTSRSYGMGWFRQAPGKEREFVSGISWRGDSTGYADSVKGRFTISRDNAKNTVDLQMNSLDPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 159 - protein, 306 aa, Unknown
  Description of Unknown TC3-068 V13A, K88T, I94Q, V268T sequence
MQVKLEESGGGSAQTGGSLRLTCAASGRTSRSYGMGWFRQAPGKEREFVSGISWRGDSTGYADSVKGRFTISRDNAKNTVDLQMNSLTPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 160 - protein, 306 aa, Unknown
  Description of Unknown TC3-069 G16D, K88T, I94Q, V268T sequence
MQVKLEESGGGSVQTDGSLRLTCAASGRTSRSYGMGWFRQAPGKEREFVSGISWRGDSTGYADSVKGRFTISRDNAKNTVDLQMNSLTPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 161 - protein, 306 aa, Unknown
  Description of Unknown TC3-070 S18D, K88T, I94Q, V268T sequence
MQVKLEESGGGSVQTGGDLRLTCAASGRTSRSYGMGWFRQAPGKEREFVSGISWRGDSTGYADSVKGRFTISRDNAKNTVDLQMNSLTPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHETVVVDDERCKKLMKQFIDERPQDWFEDIGEGSSHHHHHH

SEQ ID NO: 162 - protein, 306 aa, Unknown
  Description of Unknown TC3-071 V13A, K88D, I94R, V269T sequence
MQVKLEESGGGSAQTGGSLRLTCAASGRTSRSYGMGWFRQAPGKEREFVSGISWRGDSTGYADSVKGRFTISRDNAKNTVDLQMNSLDPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 163 - protein, 306 aa, Unknown
  Description of Unknown TC3-072 G16D, K88D, I94R, V269T sequence
MQVKLEESGGGSVQTDGSLRLTCAASGRTSRSYGMGWFRQAPGKEREFVSGISWRGDSTGYADSVKGRFTISRDNAKNTVDLQMNSLDPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 164 - protein, 306 aa, Unknown
  Description of Unknown TC3-073 S18D, K88D, I94R, V269T sequence
MQVKLEESGGGSVQTGGDLRLTCAASGRTSRSYGMGWFRQAPGKEREFVSGISWRGDSTGYADSVKGRFTISRDNAKNTVDLQMNSLDPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 165 - protein, 306 aa, Unknown
  Description of Unknown TC3-074 V13A, K88T, I94R, V269T sequence
MQVKLEESGGGSAQTGGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 166 - protein, 306 aa, Unknown
  Description of Unknown TC3-075 G16D, K88T, I94R, V269T sequence
MQVKLEESGGGSVQTDGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 167 - protein, 306 aa, Unknown
  Description of Unknown TC3-076 S18D, K88T, I94R, V269T sequence
MQVKLEESGGGSVQTGGDLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 168 - protein, 306 aa, Unknown
  Description of Unknown TC3-077 V13A, K88D, I94Q, V269T sequence
MQVKLEESGGGSAQTGGSLRLTCAASGRTSRSYGMGWFRQAPGKEREFVSGISWRGDSTGYADSVKGRFTISRDNAKNTVDLQMNSLDPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 169 - protein, 306 aa, Unknown
  Description of Unknown TC3-078 G16D, K88D, I94Q, V269T sequence
MQVKLEESGGGSVQTDGSLRLTCAASGRTSRSYGMGWFRQAPGKEREFVSGISWRGDSTGYADSVKGRFTISRDNAKNTVDLQMNSLDPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 170 - protein, 306 aa, Unknown
  Description of Unknown TC3-079 S18D, K88D, I94Q, V269T sequence
MQVKLEESGGGSVQTGGDLRLTCAASGRTSRSYGMGWFRQAPGKEREFVSGISWRGDSTGYADSVKGRFTISRDNAKNTVDLQMNSLDPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 171 - protein, 306 aa, Unknown
  Description of Unknown TC3-080 V13A, K88T, I94Q, V269T sequence
MQVKLEESGGGSAQTGGSLRLTCAASGRTSRSYGMGWFRQAPGKEREFVSGISWRGDSTGYADSVKGRFTISRDNAKNTVDLQMNSLTPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 172 - protein, 306 aa, Unknown
  Description of Unknown TC3-081 G16D, K88T, I94Q, V269T sequence
MQVKLEESGGGSVQTDGSLRLTCAASGRTSRSYGMGWFRQAPGKEREFVSGISWRGDSTGYADSVKGRFTISRDNAKNTVDLQMNSLTPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 173 - protein, 306 aa, Unknown
  Description of Unknown TC3-082 S18D, K88T, I94Q, V269T sequence
MQVKLEESGGGSVQTGGDLRLTCAASGRTSRSYGMGWFRQAPGKEREFVSGISWRGDSTGYADSVKGRFTISRDNAKNTVDLQMNSLTPEDTAQYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGEGSSHHHHHH

SEQ ID NO: 174 - protein, 291 aa, Unknown
  Description of Unknown TC3-084 h7D12v2-CDoem3-H sequence
MEVQLVESGGGLVQTGRSLRLSCAASGRTSRSYGMGWFRQAPGKEREFVSGISWRGDSTGYADSVKGRFTISRDNAKNSVYLQMNSLRAEDTALYYCAAAAGSAWYGTLYEYDYWGQGTLVTVSSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 175 - protein, 291 aa, Unknown
  Description of Unknown TC3-083 h7D12v1-CDoem3-H sequence
MQVQLVESGGGLVQTGRSLRLSCAASGRTSRSYGMGWFRQAPGKEREFVSGISWRGDSTGYADSVKGRFTISRDNAKNSVYLQMNSLRAEDTAIYYCAAAAGSAWYGTLYEYDYWGQGTLVTVSSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 176 - protein, 291 aa, Unknown
  Description of Unknown TC3-085 h7D12v3-CDoem3-H sequence
MEVQLVESGGGLVQPGRSLRLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 177 - protein, 291 aa, Unknown
  Description of Unknown TC3-086 h7D12v4-CDoem3-H sequence
MEVQLVESGGGLVQPGRSLRLSCAASGFTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGEGSSHHHHHH

SEQ ID NO: 178 - protein, 306 aa, Unknown
  Description of Unknown TC3-087 S12V, T15V, K88R, P89A, I94V, V268A sequence
MQVKLEESGGGVVQVGGSLRLTCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEATVVDDERCKKLMKQFIDERPQDWFEDIGEGSSHHHHHH

SEQ ID NO: 179 - protein, 306 aa, Unknown
  Description of Unknown TC3-088 S12V, T15V, K88R, P89A, I94V, Y224H, V268A sequence
MQVKLEESGGGVVQVGGSLRLTCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLHTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 180 - protein, 306 aa, Unknown
  Description of Unknown TC3-089 V13A, K88T, I94R, V268A, V269T sequence
MQVKLEESGGGSAQTGGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEATVVDDERCKKLMKQFIDERPQDWFEDIGEGSSHHHHHH

SEQ ID NO: 181 - protein, 306 aa, Unknown
  Description of Unknown TC3-090 V13A, K88T, I94R, Y224H, V268A sequence
MQVKLEESGGGSAQTGGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLHTTLSPCDMCTGAIIMYGIPRCVIGENVNFKSKGEKYLQTRGHEAVVVDDERCKKLMKQFIDERPQDWFEDIGEGSSHHHHHH

SEQ ID NO: 182 - protein, 297 aa, Unknown
  Description of Unknown TC4_44 (S12V, T15V, K88R, P89A, I94V, V268A) sequence
MQVKLEESGGGVVQVGGSLRLTCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGE

SEQ ID NO: 183 - protein, 297 aa, Unknown
  Description of Unknown TC4_50 (V13A, K88T, I94R, V268A) sequence
MQVKLEESGGGSAQTGGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGE

SEQ ID NO: 184 - protein, 297 aa, Unknown
  Description of Unknown TC4_51 (G16D, K88T, I94R, V268A) sequence
MQVKLEESGGGSVQTDGSLRLTCAASGRTSRSYGMGWFRQAPGKEREFVSGISWRGDSTGYADSVKGRFTISRDNAKNTVDLQMNSLTPEDTARYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGE

SEQ ID NO: 185 - protein, 297 aa, Unknown
  Description of Unknown TC4_87 (S12V, T15V, K88R, P89A, I94V, V268A, V269T) sequence
MQVKLEESGGGVVQVGGSLRLTCAASGRTSRSYGMGWFRQAPGKEREFVSGISWRGDSTGYADSVKGRFTISRDNAKNTVDLQMNSLRAEDTAVYYCAAAAGSAWYGTLYEYDYWGQGTQVTVSSGGGGSGGGGSGGGGSVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEATVVDDERCKKLMKQFIDERPQDWFEDIGE

SEQ ID NO: 186 - protein, 158 aa, Unknown
  Description of Unknown Protein sequence of yeast cytosine deaminase WITH methionine
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 187 - protein, 158 aa, Unknown
  Description of Unknown Protein sequence of mutant yeast cytosine deaminase WITH methionine
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVVVVDDERCKKLMKQFIDERPQDWFEDIGE

SEQ ID NO: 188 - protein, 15 aa, Artificial Sequence
  Description of Artificial Sequence Synthetic peptide
GGGGSGGGGSGGGGS

SEQ ID NO: 189 - protein, 157 aa, Unknown
  Description of Unknown Protein sequence of Cdoem V128A WITHOUT methionine
VTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGE

SEQ ID NO: 190 - protein, 158 aa, Unknown
  Description of Unknown Protein sequence of Cdoem V129A WITH methionine
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEAVVVDDERCKKLMKQFIDERPQDWFEDIGE

SEQ ID NO: 191 - protein, 157 aa, Unknown
  Description of Unknown Protein sequence of Cdoem V129T WITHOUT methionine
VTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGE

SEQ ID NO: 192 - protein, 158 aa, Unknown
  Description of Unknown Protein sequence of Cdoem V130T WITH methionine
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEVTVVDDERCKKLMKQFIDERPQDWFEDIGE

SEQ ID NO: 193 - protein, 157 aa, Unknown
  Description of Unknown Protein sequence of Cdoem V128A, V129T WITHOUT methionine
VTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEATVVDDERCKKLMKQFIDERPQDWFEDIGE

SEQ ID NO: 194 - protein, 158 aa, Unknown
  Description of Unknown Protein sequence of Cdoem V129A, V130T WITH methionine
MVTGGMASKWDQKGMDIAYEEALLGYKEGGVPIGGCLINNKDGSVLGRGHNMRFQKGSATLHGEISTLENCGRLEGKVYKDTTLYTTLSPCDMCTGAIIMYGIPRCVIGENVNFKSKGEKYLQTRGHEATVVDDERCKKLMKQFIDERPQDWFEDIGE

SEQ ID NO: 195 - DNA, 897 bp, Unknown
  Description of Unknown Polynucleotide sequence encodes TC4_44 (S12V, T15V, K88R, P89A, I94V, V268A)
atgcaggtgaaactggaagaaagcggtggtggtgttgttcaggttggtggtagcctgcgtctgacctgtgcagcaagcggtcgtaccagccgtagctatggtatgggttggtttcgtcaggcaccgggtaaagaacgtgaatttgttagcggtattagctggcgtggtgatagcaccggttatgcagatagcgttaaaggtcgttttaccattagccgtgataatgccaaaaataccgttgacctgcagatgaatagcctgcgtgcagaagataccgcagtttattattgtgcagcagcagccggtagcgcctggtatggcaccctgtatgaatatgattattggggtcagggcacccaggttaccgttagctcaggtggtggtggcagcggtggcggtggttcaggtggcggaggtagcgttaccggtggtatggcaagcaaatgggatcagaaaggtatggatattgcatatgaagaagcactgctgggttataaagaaggtggcgttccgattggtggttgtctgattaataacaaagatggtagcgttctgggtcgcggtcataatatgcgctttcagaaaggcagcgcaaccctgcatggtgaaattagcaccctggaaaattgtggtcgtctggaaggtaaagtgtataaagataccaccctgtataccacactgagcccgtgtgatatgtgtaccggtgccattattatgtatggtattccgcgttgtgtgattggcgaaaacgttaactttaaaagcaaaggcgagaaatatctgcagacccgtggtcatgaagcagttgttgtggatgatgaacgttgcaaaaaactgatgaaacagtttatcgatgaacgtccgcaggattggtttgaagatattggtgaataataa

SEQ ID NO: 196 - DNA, 897 bp, Unknown
  Description of Unknown Polynucleotide sequence encodes TC4_50 (V13A, K88T, I94R, V268A)
atgcaggtgaaactggaagaaagcggtggtggtagcgcacagaccggtggtagcctgcgtctgacctgtgcagcaagcggtcgtaccagccgtagctatggtatgggttggtttcgtcaggcaccgggtaaagaacgtgaatttgttagcggtattagctggcgtggtgatagcaccggttatgcagatagcgttaaaggtcgttttaccattagccgtgataatgccaaaaataccgttgacctgcagatgaatagcctgaccccggaagataccgcacgttattattgtgcagcagcagccggtagcgcctggtatggcaccctgtatgaatatgattattggggtcagggcacccaggttaccgttagctcaggtggtggtggcagcggtggcggtggttcaggtggcggaggtagcgttaccggtggtatggcaagcaaatgggatcagaaaggtatggatattgcatatgaagaagcactgctgggttataaagaaggtggcgttccgattggtggttgtctgattaataacaaagatggtagcgttctgggtcgcggtcataatatgcgctttcagaaaggcagcgcaaccctgcatggtgaaattagcaccctggaaaattgtggtcgtctggaaggtaaagtgtataaagataccaccctgtataccacactgagcccgtgtgatatgtgtaccggtgccattattatgtatggtattccgcgttgtgtgattggcgaaaacgttaactttaaaagcaaaggcgagaaatatctgcagacccgtggtcatgaagcagttgttgtggatgatgaacgttgcaaaaaactgatgaaacagtttatcgatgaacgtccgcaggattggtttgaagatattggtgaataataa

SEQ ID NO: 197 - DNA, 897 bp, Unknown
  Description of Unknown Polynucleotide sequence encodes TC4_51 (G16D, K88T, I94R, V268A)
atgcaggtgaaactggaagaaagcggtggtggtagcgttcagaccgatggtagcctgcgtctgacctgtgcagcaagcggtcgtaccagccgtagctatggtatgggttggtttcgtcaggcaccgggtaaagaacgtgaatttgttagcggtattagctggcgtggtgatagcaccggttatgcagatagcgttaaaggtcgttttaccattagccgtgataatgccaaaaataccgttgacctgcagatgaatagcctgaccccggaagataccgcacgttattattgtgcagcagcagccggtagcgcctggtatggcaccctgtatgaatatgattattggggtcagggcacccaggttaccgttagctcaggtggtggtggcagcggtggcggtggttcaggtggcggaggtagcgttaccggtggtatggcaagcaaatgggatcagaaaggtatggatattgcatatgaagaagcactgctgggttataaagaaggtggcgttccgattggtggttgtctgattaataacaaagatggtagcgttctgggtcgcggtcataatatgcgctttcagaaaggcagcgcaaccctgcatggtgaaattagcaccctggaaaattgtggtcgtctggaaggtaaagtgtataaagataccaccctgtataccacactgagcccgtgtgatatgtgtaccggtgccattattatgtatggtattccgcgttgtgtgattggcgaaaacgttaactttaaaagcaaaggcgagaaatatctgcagacccgtggtcatgaagcagttgttgtggatgatgaacgttgcaaaaaactgatgaaacagtttatcgatgaacgtccgcaggattggtttgaagatattggtgaataataa

SEQ ID NO: 198 - DNA, 897 bp, Unknown
  Description of Unknown Polynucleotide sequence encodes TC4_87 (S12V, T15V, K88R, P89A, I94V, V268A, V269T)
atgcaggtgaaactggaagaaagcggtggtggtgttgttcaggttggtggtagcctgcgtctgacctgtgcagcaagcggtcgtaccagccgtagctatggtatgggttggtttcgtcaggcaccgggtaaagaacgtgaatttgttagcggtattagctggcgtggtgatagcaccggttatgcagatagcgttaaaggtcgttttaccattagccgtgataatgccaaaaataccgttgacctgcagatgaatagcctgcgtgcagaagataccgcagtttattattgtgcagcagcagccggtagcgcctggtatggcaccctgtatgaatatgattattggggtcagggcacccaggttaccgttagctcaggtggtggtggcagcggtggcggtggttcaggtggcggaggtagcgttaccggtggtatggcaagcaaatgggatcagaaaggtatggatattgcatatgaagaagcactgctgggttataaagaaggtggcgttccgattggtggttgtctgattaataacaaagatggtagcgttctgggtcgcggtcataatatgcgctttcagaaaggcagcgcaaccctgcatggtgaaattagcaccctggaaaattgtggtcgtctggaaggtaaagtgtataaagataccaccctgtataccacactgagcccgtgtgatatgtgtaccggtgccattattatgtatggtattccgcgttgtgtgattggcgaaaacgttaactttaaaagcaaaggcgagaaatatctgcagacccgtggtcatgaagcaaccgttgtggatgatgaacgttgcaaaaaactgatgaaacagtttatcgatgaacgtccgcaggattggtttgaagatattggtgaataataa

SEQ ID NO: 199 - protein, 5 aa, Unknown
  Description of Unknown 2D3 CDR1 sequence
DYAMS

SEQ ID NO: 200 - protein, 17 aa, Unknown
  Description of Unknown 2D3 CDR2 sequence
SINWSGTHTDYADSVKG

SEQ ID NO: 201 - protein, 15 aa, Unknown
  Description of Unknown 2D3 CDR3 sequence
NWRDAGTTWFEKSGS

SEQ ID NO: 202 - protein, 5 aa, Unknown
  Description of Unknown 5F7 CDR1 sequence
INTMG

SEQ ID NO: 203 - protein, 16 aa, Unknown
  Description of Unknown 5F7 CDR2 sequence
LISSIGDTYYADSVKG

SEQ ID NO: 204 - protein, 10 aa, Unknown
  Description of Unknown 5F7 CDR3 sequence
FRTAAQGTDY

SEQ ID NO: 205 - protein, 5 aa, Unknown
  Description of Unknown 47D5 CDR1 sequence
FNDMA

SEQ ID NO: 206 - protein, 16 aa, Unknown
  Description of Unknown 47D5 CDR2 sequence
LISRVGVTSSADSVKG

SEQ ID NO: 207 - protein, 11 aa, Unknown
  Description of Unknown 47D5 CDR3 sequence
DQRLDGSTLAY

SEQ ID NO: 208 - protein, 5 aa, Unknown
  Description of Unknown BCD090-M2 CDR1 sequence
MYTMG

SEQ ID NO: 209 - protein, 17 aa, Unknown
  Description of Unknown BCD090-M2 CDR2 sequence
ANRGRGLSPDIADSVNG

SEQ ID NO: 210 - protein, 18 aa, Unknown
  Description of Unknown BCD090-M2 CDR3 sequence
DLQYGSSWPQRSSAEYDY

SEQ ID NO: 211 - protein, 5 aa, Unknown
  Description of Unknown anti-CEA vhh (ABS29544.1) CDR1 sequence
PYRMA

SEQ ID NO: 212 - protein, 18 aa, Unknown
  Description of Unknown anti-CEA vhh (ABS29544.1) CDR2 sequence
DISSGDGRTTNYADFAKG

SEQ ID NO: 213 - protein, 10 aa, Unknown
  Description of Unknown anti-CEA vhh (ABS29544.1) CDR3 sequence
FVSFVGIARS

SEQ ID NO: 214 - protein, 5 aa, Unknown
  Description of Unknown NbCEA5 CDR1 sequence
SYWMG

SEQ ID NO: 215 - protein, 17 aa, Unknown
  Description of Unknown NbCEA5 CDR2 sequence
AINRGGGYTVYADSVKG

SEQ ID NO: 216 - protein, 14 aa, Unknown
  Description of Unknown NbCEA5 CDR3 sequence
SGVLGGLHEDWFNY

(19) United States
(12) Patent Application Publication
(10) Pub. No.: US 2019/0202931 A1
(43) Pub. Date: Jul. 4, 2019

(54) SINGLE-DOMAIN ANTIBODY-CYTOSINE DEAMINASE FUSION PROTEINS

(71) Applicants: LUMOSA THERAPEUTICS, CO., LTD., Taipei (TW); SHANGHAI LUMOSA THERAPEUTICS, CO., LTD., Shanghai (CN)

(72) Inventors: Pei-Jiun CHEN, Taipei (TW); Shu-Hua LEE, Taipei (TW)

(73) Assignees: LUMOSA THERAPEUTICS, CO., LTD., Taipei (TW); SHANGHAI LUMOSA THERAPEUTICS, CO., LTD., Shanghai (CN)

(21) Appl. No.: 16/239,887

(22) Filed: Jan. 4, 2019

Related U.S. Application Data
(60) Provisional application No. 62/613,653, filed on Jan. 4, 2018.

(51) Int. Cl. C07K 16/32; C07K 16/28; C12N 9/78; C07K 14/725; A61P 35/00

(52) U.S. Cl. CPC C07K 16/32; C07K 16/2887; C12N 9/78; C12Y 305/04001; C07K 14/7051; A61K 38/00; C07K 16/2863; C07K 2317/569; C07K 2319/30; C07K 2317/565; A61P 35/00

(57) ABSTRACT

The disclosure relates to fusion proteins, methods of making fusion proteins, and methods of using fusion proteins, wherein the fusion proteins comprise a functional single-domain antibody (sdAb) or a functional variant thereof and a cytosine deaminase (CD) protein or a functional variant thereof, optionally connected via a peptide linker. The fusion proteins of the disclosure also have CD activity. The disclosure also relates to pharmaceutical compositions or formulations comprising such fusion proteins and pharmaceutically acceptable excipients, as well as medical uses of these fusion proteins.

### RELATED APPLICATIONS

This application claims priority to U.S. Provisional Application No. 62/613,653, filed Jan. 4, 2018, which is incorporated by reference in its entirety.

### REFERENCE TO SEQUENCE LISTING SUBMITTED ELECTRONICALLY

An official copy of the sequence listing is submitted electronically via EFS-Web as an ASCII formatted sequence listing with a file named 13075_0003_SL.txt, created on Jan. 3, 2019, and having a size of 385,734 bytes. The sequence listing contained in this ASCII formatted document is part of the specification and is herein incorporated by reference in its entirety.

### FIELD

The disclosure relates to fusion proteins, methods of making fusion proteins, and methods of using fusion proteins. More specifically, the disclosure relates to fusion proteins comprising a functional single-domain antibody (sdAb) (e.g., a VHH or a nanobody), or a functional variant thereof, against a cellular protein and a cytosine deaminase (CD) protein, or a functional variant thereof, optionally connected via a peptide linker. The sdAb or functional variant thereof may be connected to the N-terminus or to the C-terminus of the CD protein or functional variant thereof, optionally via a peptide linker. The fusion proteins of the disclosure also have CD activity. The disclosure also relates to pharmaceutical compositions or formulations comprising such fusion proteins and pharmaceutically acceptable excipients, as well as medical uses of these fusion proteins.

### BACKGROUND

The chemotherapeutic drug 5-fluorouracil (5-FU) has been widely used in cancer treatment, but systemic therapy with 5-FU is associated with severe toxic side effects. This agent inhibits cell growth by interfering with transcription and can be useful in the treatment of cancer and other proliferative diseases. Cytosine deaminase (CD) converts the non-toxic prodrug 5-fluorocytosine (5-FC) to the cytotoxic agent 5-fluorouracil. 5-FC is not toxic to human cells because of the lack of CD in human cells. The cytosine deaminase gene, co-administered with the 5-fluorocytosine prodrug, is one of the most widely tested suicide systems in cancer gene therapy. Expression of a CD gene within a tumor can induce, after 5-FC treatment of the subject, the local production of 5-fluorouracil resulting in intratumor chemotherapy. Combining the administration of 5-FC and a CD is often associated with systemic toxicity. There is therefore a need for alternative means for utilizing the CD/5-FC combination strategy in the treatment of proliferative diseases.

Antibody-directed enzyme-prodrug therapy (ADEPT) has been developed to overcome this limitation. The activating enzyme, conjugated to a tumor-specific antibody, is delivered into tumor cells, followed by administration of the prodrug that is inert until it is activated by the enzyme. Thus, systemic toxicity can be avoided.

Park et al. disclosed a recombinant fusion protein containing the hyaluronan binding domain of TSG-6 (Link) and yeast cytosine deaminase (CD). In their studies, various Link-CD constructs were expressed in BL21-Codon Plus® (DE3) RIPL E. coli cells, such as GST-tag, (Gly4Ser)3 (SEQ ID NO: 188) linker. Most of the expressed proteins aggregated in the inclusion bodies, despite efforts to increase accumulation of soluble protein. On average, about 500 μg/L of the purified protein was recovered from the soluble fraction per liter culture medium (Mol Pharm. 2009; 6(3): 801-812).

Deckert et al. disclosed an A33scFv-cytosine deaminase recombinant protein and demonstrated specific antigen binding and enzyme activity thereof. A33scFv-CD was expressed by a T7-RNA polymerase-controlled bacterial system using BL21 Escherichia coli λDE3 lysogens. Fusion proteins were expressed as inclusion bodies with a final culture yield of about 100 μg/L (British Journal of Cancer (2003) 88, 937-939).

Expressing a heterogeneous protein consisting of human antibody sequences and bacterial enzyme in a single expression system is difficult. Coelho et al. attempted to produce A33scFv-cytosine deaminase recombinant protein in Pichia pastoris to select a high yield clone for production. In their studies, a high-producing pPICZαA-transformant Pichia clone was selected and the target protein can be secreted into culture supernatant. The total yield after purification was about 1.0 mg/L (International Journal of Oncology 31: 951-957, 2007).

These results all indicated that fusions of a tumor-specific antibody or antigen-binding fragment with CD are not suitable for industrial production. Therefore, there is a need for CD fusion proteins that have promising production yield for industrial use and that demonstrate specific antigen binding and enzyme activity.

### SUMMARY

This disclosure provides fusion proteins, methods of making fusion proteins, and methods of using fusion proteins. The following are non-limiting exemplary embodiments of the disclosure.

In some embodiments of the disclosure, the fusion protein comprises formula I or formula II:

N-(L)n-C (formula I);

C-(L)n-N (formula II);

wherein N is a single-domain antibody (sdAb) or a functional variant thereof, L is a peptide linker and n=0-50, and C is a cytosine deaminase (CD) protein or a functional variant thereof.

In some aspects of these embodiments, the fusion protein comprises formula I and the C-terminus of a peptide linker or the C-terminus of a sdAb or functional variant thereof is fused to the N-terminus of a CD protein or functional variant thereof. For example, in some aspects, a peptide linker is not present and the C-terminus of a sdAb or functional variant thereof is fused to the N-terminus of a CD protein or functional variant thereof. In some aspects, at least one peptide linker is present and the C-terminus of a sdAb or functional variant thereof is fused to the N-terminus of a peptide linker, and the C-terminus of a peptide linker is fused to the N-terminus of another peptide linker or to the N-terminus of a CD protein or functional variant thereof.

In other aspects of these embodiments, the fusion protein comprises formula II and the C-terminus of a peptide linker or the C-terminus of a CD protein or functional variant thereof is fused to the N-terminus of a sdAb or functional variant thereof. For example, in some aspects, a peptide linker is not present and the C-terminus of a CD protein or functional variant thereof is fused to the N-terminus of a sdAb or functional variant thereof. In some aspects, at least one peptide linker is present and the C-terminus of a CD protein or functional variant thereof is fused to the N-terminus of a peptide linker, and the C-terminus of a peptide linker is fused to the N-terminus of another peptide linker or to the N-terminus of a sdAb or functional variant thereof.

In some embodiments, the fusion protein consists essentially of formula I. In some embodiments, the fusion protein is formula I.

In some embodiments, the fusion protein consists essentially of formula II. In some embodiments, the fusion protein is formula II.

In some embodiments, the sdAb or functional variant thereof binds to a target, wherein the target is selected from the group consisting of a cell membrane molecule, a secreted molecule, and an intracellular molecule. In some embodiments, the target is a tumor-associated antigen or a tumor-specific antigen.

In some embodiments, the target is selected from the group consisting of EGFR, 5T4, A33, AFP, Beta-catenin, BRCA1, BRCA2, C242, CCR4, CD152, CD19, CD20, CD200, CD22, CD221, CD23, CD30, CD3, CD37, CD40, CD44, CD5, CD51, CD52, CD56, CD64, CD74, CD80, CDCP1, c-KIT, COX-2, cMET, CSF1R, CTLA-4, EGF2, ErbB2, ErbB3, FGFR1, FGFR2, FGFR3, FLT3, HER2, HER3, HIF-Ia, HLA-DR, IGF-IR, mTOR, NPC-1C, P53, PDGFRα, PDGFRβ, PLGF, PSA, RGMa, RoN, TNF, TP53, TPD52, VEGFR1, VEGFR2, VEGFR3, CA-IX, αvβ3, α5β1, FAP, glycoprotein 75, TAG-72, MUC16, NR-LU-13, SLAMF7, EGP40, BAFF, PRL-3, carcinoembryonic antigen (CEA), prostate-specific membrane antigen, MART-1, gp100, Cancer-testis (CT) antigens (e.g. NY-ESO-1, MAGE-A3, MAGE-A1), hTERT, Mesothelin, MCC, Mum-1, ERBB2IP, EpCAM, TfR, integrin α6β4, HGFR, PTP-LAR, CD147, CDCP1, CEACAM6, JAM1, integrin α3β1, integrin αvβ3, PD-L1, AXL, CDH6, DLL3, EDNRB, EFNA4, NEPP3, EPHA2, FOLR1, LewisY, GPNMB, GUCY2C, HAVCR1, Integrin α, LYPD3, MUC1, NECTIN4, NOTCH3, PTK7, SLC34A2, SLC39A6, SLC44A4, SLITRK6, STEAP1, TACSTD2, TPBG, TIM-1, GD2, and nicotinic acetylcholine receptor (nAChR).

In some embodiments, the target is selected from the group consisting of EGFR, c-KIT, cMET, HER2, HER3, FGFR1, FGFR2, FGFR3, IGF-IR, P53, PDGFRα, VEGFR1, VEGFR2, VEGFR3, CA-IX, αvβ3, α5β1, MUC16, carcinoembryonic antigen (CEA), prostate-specific membrane antigen, Cancer-testis (CT) antigens (e.g., NY-ESO-1, MAGE-A3, MAGE-A1), Mesothelin, EpCAM, integrin α6β4, CEACAM6, integrin α3β1, integrin αvβ3, PD-L1, AXL, CDH6, DLL3, EDNRB, EFNA4, NEPP3, EPHA2, FOLR1, LewisY, GPNMB, GUCY2C, HAVCR1, Integrin α, LYPD3, MUC1, NECTIN4, NOTCH3, PTK7, SLC34A2, SLC39A6, SLC44A4, SLITRK6, STEAP1, TACSTD2, TPBG, TIM-1, GD2, and nicotinic acetylcholine receptor (nAChR).

In some embodiments, the target is VEGFR2, EGFR, CEA, HER2, or HER3.

In some embodiments, the target is VEGFR2. In some embodiments, the target is EGFR.

In some embodiments, the sdAb or functional variant thereof comprises a complementarity determining region 1 (CDR1) selected from the group consisting of SEQ ID NOs: 28, 31, and 34; a CDR2 selected from the group consisting of SEQ ID NOs: 29, 32, and 35; and a CDR3 selected from the group consisting of SEQ ID NOs: 30, 33, and 36. In some aspects, the sdAb or functional variant thereof consists essentially of a CDR1 selected from the group consisting of SEQ ID NOs: 28, 31, and 34; a CDR2 selected from the group consisting of SEQ ID NOs: 29, 32, and 35; and a CDR3 selected from the group consisting of SEQ ID NOs: 30, 33, and 36. In some aspects, the sdAb or functional variant thereof consists of a CDR1 selected from the group consisting of SEQ ID NOs: 28, 31, and 34; a CDR2 selected from the group consisting of SEQ ID NOs: 29, 32, and 35; and a CDR3 selected from the group consisting of SEQ ID NOs: 30, 33, and 36.

In some embodiments, the sdAb or functional variant thereof comprises the amino acid sequence of SEQ ID NO: 23 (3VGR19). In some aspects, the sdAb or functional variant thereof consists essentially of the amino acid sequence of SEQ ID NO: 23 (3VGR19). In some aspects, the sdAb or functional variant thereof consists of the amino acid sequence of SEQ ID NO: 23 (3VGR19).

In some embodiments, the sdAb or functional variant thereof comprises the amino acid sequence of SEQ ID NO: 24 (4VGR17). In some aspects, the sdAb or functional variant thereof consists essentially of the amino acid sequence of SEQ ID NO: 24 (4VGR17). In some aspects, the sdAb or functional variant thereof consists of the amino acid sequence of SEQ ID NO: 24 (4VGR17). In some embodiments, the sdAb or functional variant thereof comprises the amino acid sequence of SEQ ID NO: 25 (4VGR38). In some aspects, the sdAb or functional variant thereof consists essentially of the amino acid sequence of SEQ ID NO: 25 (4VGR38). In some aspects, the sdAb or functional variant thereof consists of the amino acid sequence of SEQ ID NO: 25 (4VGR38).

In some embodiments, the sdAb or functional variant thereof comprises a CDR1 selected from the group consisting of SEQ ID NOs: 37 and 40, a CDR2 selected from the group consisting of SEQ ID NOs: 38 and 41, and a CDR3 selected from the group consisting of SEQ ID NOs: 39 and 42. In some aspects, the sdAb or functional variant thereof consists essentially of a CDR1 selected from the group consisting of SEQ ID NOs: 37 and 40, a CDR2 selected from the group consisting of SEQ ID NOs: 38 and 41, and a CDR3 selected from the group consisting of SEQ ID NOs: 39 and 42. In some aspects, the sdAb or functional variant thereof consists of a CDR1 selected from the group consisting of SEQ ID NOs: 37 and 40, a CDR2 selected from the group consisting of SEQ ID NOs: 38 and 41, and a CDR3 selected from the group consisting of SEQ ID NOs: 39 and 42.

In some embodiments, the sdAb or functional variant thereof comprises the amino acid sequence of SEQ ID NO: 26 (VHH122). In some aspects, the sdAb or functional variant thereof consists essentially of the amino acid sequence of SEQ ID NO: 26 (VHH122). In some aspects, the sdAb or functional variant thereof consists of the amino acid sequence of SEQ ID NO: 26 (VHH122). In some embodiments, the sdAb or functional variant thereof comprises the amino acid sequence of SEQ ID NO: 27 (7D12). In some aspects, the sdAb or functional variant thereof consists essentially of the amino acid sequence of SEQ ID NO: 27 (7D12). In some aspects, the sdAb or functional variant thereof consists of the amino acid sequence of SEQ ID NO: 27 (7D12).

In some embodiments, the sdAb or functional variant thereof comprises a CDR1 selected from the group consisting of SEQ ID NOs: 199, 202, and 205; a CDR2 selected from the group consisting of SEQ ID NOs: 200, 203, and 206; and a CDR3 selected from the group consisting of SEQ ID NOs: 201, 204, and 207. In some embodiments, the sdAb or functional variant thereof consists essentially of a CDR1 selected from the group consisting of SEQ ID NOs: 199, 202, and 205; a CDR2 selected from the group consisting of SEQ ID NOs: 200, 203, and 206; and a CDR3 selected from the group consisting of SEQ ID NOs: 201, 204, and 207. In some embodiments, the sdAb or functional variant thereof consists of a CDR1 selected from the group consisting of SEQ ID NOs: 199, 202, and 205; a CDR2 selected from the group consisting of SEQ ID NOs: 200, 203, and 206; and a CDR3 selected from the group consisting of SEQ ID NOs: 201, 204, and 207.

In some embodiments, the sdAb or functional variant thereof comprises the amino acid sequence of SEQ ID NO: 69 (2D3). In some aspects, the sdAb or functional variant thereof consists essentially of the amino acid sequence of SEQ ID NO: 69 (2D3). In some aspects, the sdAb or functional variant thereof consists of the amino acid sequence of SEQ ID NO: 69 (2D3). In some embodiments, the sdAb or functional variant thereof comprises the amino acid sequence of SEQ ID NO: 70 (5F7). In some aspects, the sdAb or functional variant thereof consists essentially of the amino acid sequence of SEQ ID NO: 70 (5F7). In some aspects, the sdAb or functional variant thereof consists of the amino acid sequence of SEQ ID NO: 70 (5F7). In some embodiments, the sdAb or functional variant thereof comprises the amino acid sequence of SEQ ID NO: 71 (47D5). In some aspects, the sdAb or functional variant thereof consists essentially of the amino acid sequence of SEQ ID NO: 71 (47D5). In some aspects, the sdAb or functional variant thereof consists of the amino acid sequence of SEQ ID NO: 71 (47D5).

In some embodiments, the sdAb or functional variant thereof comprises a CDR1 of SEQ ID NO: 208, a CDR2 of SEQ ID NO: 209, and a CDR3 of SEQ ID NO: 210. In some embodiments, the sdAb or functional variant thereof consists essentially of a CDR1 of SEQ ID NO: 208, a CDR2 of SEQ ID NO: 209, and a CDR3 of SEQ ID NO: 210. In some embodiments, the sdAb or functional variant thereof consists of a CDR1 of SEQ ID NO: 208, a CDR2 of SEQ ID NO: 209, and a CDR3 of SEQ ID NO: 210.

In some embodiments, the sdAb or functional variant thereof comprises the amino acid sequence of SEQ ID NO: 75 (BCD090-M2). In some aspects, the sdAb or functional variant thereof consists essentially of the amino acid sequence of SEQ ID NO: 75 (BCD090-M2). In some aspects, the sdAb or functional variant thereof consists of the amino acid sequence of SEQ ID NO: 75 (BCD090-M2).

In some embodiments, the sdAb or functional variant thereof comprises a CDR1 selected from the group consisting of SEQ ID NOs: 211 and 214, a CDR2 selected from the group consisting of SEQ ID NOs: 212 and 215, and a CDR3 selected from the group consisting of SEQ ID NOs: 213 and 216. In some embodiments, the sdAb or functional variant thereof consists essentially of a CDR1 selected from the group consisting of SEQ ID NOs: 211 and 214, a CDR2 selected from the group consisting of SEQ ID NOs: 212 and 215, and a CDR3 selected from the group consisting of SEQ ID NOs: 213 and 216. In some embodiments, the sdAb or functional variant thereof consists of a CDR1 selected from the group consisting of SEQ ID NOs: 211 and 214, a CDR2 selected from the group consisting of SEQ ID NOs: 212 and 215, and a CDR3 selected from the group consisting of SEQ ID NOs: 213 and 216.

In some embodiments, the sdAb or functional variant thereof comprises the amino acid sequence of SEQ ID NO: 77 (ABS29544.1). In some aspects, the sdAb or functional variant thereof consists essentially of the amino acid sequence of SEQ ID NO: 77 (ABS29544.1). In some aspects, the sdAb or functional variant thereof consists of the amino acid sequence of SEQ ID NO: 77 (ABS29544.1). In some embodiments, the sdAb or functional variant thereof comprises the amino acid sequence of SEQ ID NO: 79 (NbCEA5). In some aspects, the sdAb or functional variant thereof consists essentially of the amino acid sequence of SEQ ID NO: 79 (NbCEA5). In some aspects, the sdAb or functional variant thereof consists of the amino acid sequence of SEQ ID NO: 79 (NbCEA5).

In some embodiments, at least one peptide linker is present and comprises the amino acid sequence (GGGGS)n (SEQ ID NO: 1), wherein n is 1, 2, 3, 4, 5, or 6. In some embodiments, at least one peptide linker is present and comprises the amino acid sequence of SEQ ID NO: 4 or SEQ ID NO: 5. In some embodiments, at least one peptide linker is present and comprises the amino acid sequence (GGGGS)3 (SEQ ID NO: 188).

In some embodiments, the CD protein or functional variant thereof is a bacterial CD protein or a functional variant thereof or a yeast CD protein or a functional variant thereof.

In some embodiments, the CD protein or functional variant thereof comprises an amino acid sequence selected from the group consisting of SEQ ID NOs: 21, 22, 186, and 187. In some aspects, the CD protein or functional variant thereof consists essentially of an amino acid sequence selected from the group consisting of SEQ ID NOs: 21, 22, 186, and 187. In some aspects, the CD protein or functional variant thereof consists of an amino acid sequence selected from the group consisting of SEQ ID NOs: 21, 22, 186, and 187. In some aspects, the CD comprises, consists essentially of, or consists of the amino acid sequence of SEQ ID NO: 22. In some aspects, the CD comprises, consists essentially of, or consists of the amino acid sequence of SEQ ID NO: 187.

In some embodiments, the CD protein or functional variant thereof comprises an amino acid sequence that is at least 90% identical, at least 91% identical, at least 92% identical, at least 93% identical, at least 94% identical, at least 95% identical, at least 96% identical, at least 97% identical, at least 98% identical, at least 99% identical, or 100% identical to any one of SEQ ID NOs: 21, 22, 186, or 187.

In some embodiments, the fusion protein comprises a functional variant of a CD protein having the starting amino acid sequence of SEQ ID NO: 21 or 22, wherein the functional variant comprises at least one mutation compared to the starting sequence selected from the group consisting of Y84A, Y84H, T85D, T86E, M92N, M92A, M92K, M92Q, V128A, V128T, V129A, V129L, V129I, V129T, V130A, and V130T. In some embodiments, the fusion protein comprises a functional variant of a CD protein having the starting amino acid sequence of SEQ ID NO: 186 or 187, wherein the functional variant comprises at least one mutation compared to the starting sequence selected from the group consisting of Y85A, Y85H, T86D, T87E, M93N, M93A, M93K, M93Q, V129A, V129T, V130A, V130L, V130I, V130T, V131A, and V131T.

In some embodiments, the functional variant of CD comprises an amino acid sequence selected from SEQ ID NOs: 22, 187, 189, 190, 191, 192, 193, and 194. In some embodiments, the functional variant of CD consists essentially of an amino acid sequence selected from SEQ ID NOs: 22, 187, 189, 190, 191, 192, 193, and 194. In some embodiments, the functional variant of CD consists of an amino acid sequence selected from SEQ ID NOs: 22, 187, 189, 190, 191, 192, 193, and 194.

In some embodiments, the fusion protein comprises an anti-VEGFR2 sdAb or functional variant thereof and a CD protein or functional variant thereof. In some aspects of these embodiments, the fusion protein comprises an amino acid sequence selected from the group consisting of SEQ ID NO: 7, SEQ ID NO: 7 without a HIS-tag (i.e., amino acids 1-297 of SEQ ID NO: 7), SEQ ID NO: 9, SEQ ID NO: 9 without a HIS-tag (i.e., amino acids 1-297 of SEQ ID NO: 9), SEQ ID NO: 11, and SEQ ID NO: 11 without a HIS-tag (i.e., amino acids 1-297 of SEQ ID NO: 11).

In some embodiments, the fusion protein comprises an anti-EGFR sdAb or functional variant thereof and a CD protein or functional variant thereof. In some aspects of these embodiments, the fusion protein comprises an amino acid sequence selected from the group consisting of SEQ ID NO: 13, SEQ ID NO: 13 without a HIS-tag (i.e., amino acids 1-297 of SEQ ID NO: 13), SEQ ID NO: 15, SEQ ID NO: 15 without a HIS-tag (i.e., amino acids 1-297 of SEQ ID NO: 15), SEQ ID NO: 17, and SEQ ID NO: 17 without a HIS-tag (i.e., amino acids 1-297 of SEQ ID NO: 17, i.e. SEQ ID NO: 19).

In some embodiments, the fusion protein comprises an anti-HER2 sdAb or functional variant thereof and a CD protein or functional variant thereof. In some aspects of these embodiments, the fusion protein comprises an amino acid sequence selected from the group consisting of SEQ ID NO: 72, SEQ ID NO: 72 without a HIS-tag (i.e., amino acids 1-297 of SEQ ID NO: 72), SEQ ID NO: 73, SEQ ID NO: 73 without a HIS-tag (i.e., amino acids 1-291 of SEQ ID NO: 73), SEQ ID NO: 74, and SEQ ID NO: 74 without a HIS-tag (i.e., amino acids 1-292 of SEQ ID NO: 74).

In some embodiments, the fusion protein comprises an anti-HER3 sdAb or functional variant thereof and a CD protein or functional variant thereof. In some aspects of these embodiments, the fusion protein comprises an amino acid sequence of SEQ ID NO: 76 and SEQ ID NO: 76 without a HIS-tag (i.e., amino acids 1-300 of SEQ ID NO: 76).

In some embodiments, the fusion protein comprises an anti-CEA sdAb or functional variant thereof and a CD protein or functional variant thereof. In some aspects of these embodiments, the fusion protein comprises an amino acid sequence selected from the group consisting of SEQ ID NO: 78, SEQ ID NO: 78 without a HIS-tag (i.e., amino acids 1-293 of SEQ ID NO: 78), SEQ ID NO: 80, and SEQ ID NO: 80 without a HIS-tag (i.e., amino acids 1-296 of SEQ ID NO: 80).

In some embodiments, the fusion protein comprises a de-immunized sdAb (e.g., a functional variant of sdAb having one or more de-immunizing mutations) and/or a de-immunized CD (e.g., a functional variant of CD having one or more de-immunizing mutations). For example, in some embodiments, the fusion protein comprises at least one de-immunizing mutation in at least one T cell epitope selected from the group consisting of Epitope 1 (SEQ ID NO: 63), Epitope 2 (SEQ ID NO: 64), Epitope 3 (SEQ ID NO: 65), Epitope 4 (SEQ ID NO: 66), Epitope 5 (SEQ ID NO: 67), and Epitope 6 (SEQ ID NO: 68). In some embodiments, the fusion protein comprises an amino acid sequence selected from SEQ ID NOs: 93-185. In some embodiments, the fusion protein consists essentially of an amino acid sequence selected from SEQ ID NOs: 93-185. In some embodiments, the fusion protein consists of an amino acid sequence selected from SEQ ID NOs: 93-185.

In some embodiments, the fusion protein comprises amino acids 1-297 of an amino acid sequence selected from the group consisting of SEQ ID NOs: 93-181. In some embodiments, the fusion protein consists essentially of amino acids 1-297 of an amino acid sequence selected from the group consisting of SEQ ID NOs: 93-181. In some embodiments, the fusion protein consists of amino acids 1-297 of an amino acid sequence selected from the group consisting of SEQ ID NOs: 93-181.

In some embodiments the fusion protein consists essentially of an amino acid sequence selected from SEQ ID NOs: 182-185.

In some embodiments, the fusion protein comprises the amino acid sequence of SEQ ID NO: 19. In some embodiments, the fusion protein consists essentially of the amino acid sequence of SEQ ID NO: 19. In some embodiments, the fusion protein consists of the amino acid sequence of SEQ ID NO: 19.

In some embodiments, the fusion protein consists essentially of an amino acid sequence selected from SEQ ID NOs: 19, 182, 183, 184, and 185.

In some aspects, the disclosure relates to a pharmaceutical composition or pharmaceutical formulation comprising at least one fusion protein of the disclosure. In some aspects, the pharmaceutical composition or pharmaceutical formulation comprises at least one fusion protein of the disclosure and at least one pharmaceutically acceptable carrier or excipient.

In some aspects, the disclosure relates to a method of treating cancer in a subject in need thereof, the method comprising administering to the subject and effective amount of at least one fusion protein or pharmaceutical composition of the disclosure. In some embodiments, the at least one fusion protein or the pharmaceutical composition is administered parenterally.

In some embodiments, the method of treating cancer in a subject in need thereof further comprises administering to the subject an effective amount of a substrate for cytosine deaminase. In some embodiments, the substrate comprises a prodrug of 5-fluorouracil. In some embodiments, the prodrug of 5-fluorouracil is selected from the group consisting of 5-fluorocytosine (5-FC), Toca FC, analogs of 5-FC, and photoactivatable compounds, salts or esters thereof. In some embodiments, the prodrug administered to the subject is 5-FC.

In some embodiments, the cancer is selected from the group consisting of colon cancer, stomach cancer, pancreatic cancer, breast cancer, basal cell carcinoma, Bowen's disease, cervical cancer, ocular surface squamous neoplasia, melanoma, renal cell carcinoma, lung cancer, bladder cancer, gall bladder cancer, laryngeal cancer, liver cancer, thyroid cancer, salivary gland cancer, prostate cancer, colorectal cancer, head and neck cancer, cholangiocarcinoma, esophagus cancer, bone cancer, endometrial cancer, ovarian cancer, soft tissue sarcoma, and Merkel cell carcinoma. In some embodiments, the cancer is a solid tumor. In some embodiments, the solid tumor is colon cancer, colorectal cancer, pancreatic cancer, or head and neck cancer.

In some aspects, the disclosure relates to a nucleic acid molecule comprising a nucleic acid sequence encoding a fusion protein of any one of the preceding embodiments.

In some aspects, the disclosure relates to a nucleic acid molecule comprising a codon-optimized nucleic acid sequence encoding a sdAb or functional variant thereof of any fusion protein of any one of the preceding embodiments. In some aspects, the disclosure relates to a nucleic acid molecule comprising a codon-optimized nucleic acid sequence encoding a CD or functional variant thereof of any fusion protein of any one of the preceding embodiments.

In some embodiments, the nucleic acid molecule comprises the nucleic acid sequence of SEQ ID NO: 8. In some embodiments, the nucleic acid molecule comprises the nucleic acid sequence of SEQ ID NO: 10. In some embodiments, the nucleic acid molecule comprises the nucleic acid sequence of SEQ ID NO: 12. In some embodiments, the nucleic acid molecule comprises the nucleic acid sequence of SEQ ID NO: 14. In some embodiments, the nucleic acid molecule comprises the nucleic acid sequence of SEQ ID NO: 16. In some embodiments, the nucleic acid molecule comprises the nucleic acid sequence of SEQ ID NO: 18. In some embodiments, the nucleic acid molecule comprises the nucleic acid sequence of SEQ ID NO: 20. In some embodiments, the nucleic acid molecule comprises the nucleic acid sequence of SEQ ID NO: 195. In some embodiments, the nucleic acid molecule comprises the nucleic acid sequence of SEQ ID NO: 196. In some embodiments, the nucleic acid molecule comprises the nucleic acid sequence of SEQ ID NO: 197. In some embodiments, the nucleic acid molecule comprises the nucleic acid sequence of SEQ ID NO: 198.

In some aspects, the disclosure relates to a vector comprising a nucleic acid encoding a fusion protein of any of the preceding embodiments.

In some aspects, the disclosure relates to a host cell comprising a vector or nucleic acid encoding a fusion protein of any of the preceding embodiments.

In some aspects, the disclosure relates to a method of making a fusion protein according to any one of the preceding embodiments, the method comprising expressing a nucleic acid encoding the fusion protein in a host cell. In some embodiments, the host cell is engineered to improve the activity, cytoplasmic production, and/or stability of proteins having disulfide bonds.

In some embodiments, the disclosed method of making a fusion protein yields a fusion protein having disulfide bonds for which the activity, cytoplasmic production, and/or stability of the fusion protein is increased by an amount selected from 2-fold, 5-fold, 10-fold, 20-fold, 50-fold, and 100-fold as compared to the fusion protein produced by a non-engineered version of the same host cell.

In some embodiments, the host cell is a non-mammalian cell. In some embodiments, the host cell is a yeast cell or a bacterial cell. In some embodiments, the host cell is an E. coli cell, an Archaebacterial cell, or an Actinobacterial cell. In some embodiments, the host cell is an E. coli strain that provides a cytoplasmic environment for disulfide bond formation. In some aspects, the cytoplasmic environment is achieved by optimizing the thioredoxin and/or glutathione pathway, and/or by expressing cytosolic disulfide bond isomerase. In some embodiments, the host cell is an E. coli strain that constitutively expresses a chromosomal copy of a cytosolic disulfide bond isomerase. In some aspects of these embodiments, the cytosolic disulfide bond isomerase is DsbC. In some aspects, the E. coli strain is SHuffle® T7, SHuffle® T7 Express, SHuffle® Express, Origami™, or Rosetta-gami™.

### BRIEF DESCRIPTION OF DRAWINGS

The drawings depict only example embodiments of the present disclosure and, therefore, do not limit its scope.

FIG. 1A shows the protein design of a full-antibody-CD-CD fusion protein. FIG. 1B shows the protein design and expression profile of a full-antibody-CD fusion protein expressed in mammalian cells.

FIG. 2A shows the vector design of an antigen-targeting domain-CD fusion protein. FIG. 2B summarizes the expression profile and functional analysis results of tested fusion proteins; v=verified; N/D=Not detected; N/A=Not available. FIG. 2C and FIG. 2D show the expression profile of several VHH-CD fusion proteins of the disclosure. 10: E. coli culture before induction. 15: E. coli culture 5 hours post-induction. Cytosol (C): the soluble fraction of cell lysate. Inclusion body (I): the insoluble fraction of cell lysate. M: Marker.

FIG. 3 shows an SDS-PAGE analysis of several sdAb-CD fusion proteins from small scale purification. M=Marker.

FIG. 4A and FIG. 4B show size-exclusion chromatography analysis of several sdAb-CD fusion proteins.

FIG. 5A and FIG. 5B show the cytosine deaminase (CDase) activity of several sdAb-CD fusion proteins.

FIG. 6A and FIG. 6B show ELISA assays demonstrating the binding ability of sdAb-CD fusion proteins to human VEGFR2 and human EGFR (FIG. 6A) and human HER2, HER3 and CEA (FIG. 6B). OD=optical density.

FIG. 7A and FIG. 7B show the purification profile from large scale purification of adAb-CD fusion proteins 3VGR19-CD-H (FIG. 7A) and 7D12-CDome3 (FIG. 7B). LS=low speed supernatant; F1=flow through 1 of Ni Sepharose column; F2=flow through 2 of Ni-Sepharose column; M=marker; Q1=1^{st} Q-Sepharose column; Q2=2^{nd} Q-Sepharose column; QF1=flow through of 1^{st} Q-Sepharose column; QF2=flow through of 2^{nd} Q-Sepharose column; X1=fraction 1; X2=fraction 2.

FIG. 8 shows the expression profile of sdAb-CD (3VGR19-CD) fusion proteins in SHuffle® T7 Express and T7 Express cells. S=supernatant (cytosol); P=pellet (inclusion body); M=marker; I₀=E. coli culture before induction; 15=E. coli culture 5 hours post-induction. The band corresponding to the fusion protein is indicated with an arrow.

FIG. 9A (MDA-MB-231) and FIG. 9B (A431) show the results of cell-based cytotoxic assays with several fusion proteins of the disclosure in antigen-expressing cells.

FIG. 10A shows the results of cell-based cytotoxic assays with the fusion proteins CDoem3-H and 7D12-CDoem3-H in EGFR-expressing cells. FIG. 10B shows the results of cell-based cytotoxic assays with the fusion proteins CDoem3 and 7D12-CDoem3 in EGFR-expressing cells. NP=negative protein control (CDoem3-H).

FIG. 11A shows the tumor growth curve of the A431 xenograft model after treatment with 7D12-CDoem3-H and 7D12-CDoem3 in combination with 5-fluorocytosine (5-FC); FIG. 11B shows the tumor weight of the A431 xenograft model after treatment with 7D12-CDoem3-H and 7D12-CDoem3 in combination with 5-FC.

FIG. 12 shows expression profile and functional analysis results for single mutation variants of 7D12-CDoem3-H.

FIG. 13A an FIG. 13B show expression profile and functional analysis results of multi-mutation variants of 7D12-CDoem3-H.

FIG. 14 shows a summary of T cell proliferation and IL-2 ELISpot responses of 7D12-CDoem3 and 7D12-CDoem3 variants.

FIG. 15 shows the cytosine deaminase activity and the binding ability of 7D12-CDoem3 and 7D12-CDoem3 variants to human EGFR.

### DETAILED DESCRIPTION

The disclosure provides dual-function fusion proteins comprising a single-domain antibody (sdAb) or functional variant thereof with a cytosine deaminase (CD) or functional variant thereof. The fusion proteins are made using the methods described herein with good biological activity and superior yields (e.g., about 1 g/L), amenable to commercial use. The fusion proteins of the disclosure can be expressed in high yields as cytosolic proteins in E. coli, and the protein stability can be further improved in an E. coli strain that has been engineered to optimize disulfide bond formation in the cytoplasm. Also provided herein are compositions and methods of using the fusion proteins of the disclosure in the treatment of cancer and other proliferative diseases.

The disclosure demonstrates that various sdAb-CD fusion proteins according to the disclosure are stable and have cytotoxic effects on cancer cells. Soluble proteins were detected by SDS-PAGE and the relative high purity of the fusion proteins is shown by SEC-HPLC. The CD and sdAb portions of the fusion proteins both maintained their original function in all of the fusion proteins produced. CD activity was demonstrated by a 5-FC-to-5-FU conversion assay and the binding of the fusion proteins to human VEGFR2, EGFR, HER3, HER2, or CEA was shown by ELISA. Through cytotoxicity tests on cancer cell lines, the sdAb-CD fusion proteins proved their ability to target and bind VEGFR2 and EGFR and convert non-toxic 5-FC to toxic 5-FU, thereby killing the antigen expressing cancer cells. In the A431 mice xenograft model, the sdAb-CD fusion proteins proved to be functional by inhibiting and suppressing tumor growth.

Expression of several fusion proteins of the disclosure using the methods described herein resulted in fusion proteins with good biological activity and superior yields (e.g., about 1 g/L), amenable to commercial use.

The disclosure also provides de-immunized sdAb-CD fusion proteins that are stable for production and which demonstrated CD activity and antigen-binding activity.

Unless specifically stated or obvious from context, as used herein, the term “or” is understood to be inclusive. Unless specifically stated or obvious from context, as used herein, the terms “a,” “an,” and “the” are understood to be singular or plural.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone).

Unless specifically stated or obvious from context, as used herein, the term “about” is understood as within a range of normal tolerance in the art, for example within two standard deviations of the mean (e.g., the stated value). “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

Any compositions or methods provided herein can be combined with one or more of any of the other compositions and methods provided herein.

The “activity” of an enzyme is a measure of its ability to catalyze a reaction, i.e., to “function,” and may be expressed as the rate at which the product of the reaction is produced. For example, enzyme activity can be represented as the amount of product produced per unit of time or per unit of enzyme (e.g., concentration or weight), or in terms of affinity or dissociation constants. As used interchangeably herein, a “cytosine deaminase activity,” “biological activity of cytosine deaminase,” or “functional activity of a cytosine deaminase” refers to an activity exerted by CD or a functional variant thereof, or by a fusion protein of the disclosure, on a CD substrate, as determined in vivo or in vitro according to standard techniques. Assays for measuring CD activity are known in the art. For example, CD activity can be measured by determining the rate of conversion of 5-FC to 5-FU or cytosine to uracil. The detection of 5-FC, 5-FU, cytosine, and uracil can be performed by the methods described in the Examples section, by chromatography, and/or by other methods known in the art.

In this disclosure, “consisting essentially of” or “consists essentially of” allows for the presence of more than that which is recited so long as basic or novel characteristics of that which is recited is not changed by the presence of more than that which is recited, but excludes prior art embodiments. For example, a polypeptide/protein or nucleic acid sequence that “consists essentially of” a recited sequence may include one or more additional amino acids or nucleic acids, respectively, that do not destroy the biological activity of the recited sequence.

As used herein, “fusion polypeptide” or “fusion protein” refers to a protein comprising two or more different polypeptides or active fragments thereof that are not naturally present in the same protein. A fusion protein has a single contiguous polypeptide backbone, optionally with a peptide linker between any of the two or more different polypeptides. Fusion proteins can be prepared using conventional techniques in molecular biology to join the two or more genes in frame into a single nucleic acid sequence, and then expressing the nucleic acid in an appropriate host cell under conditions in which the fusion protein is produced.

As used herein, “functional variant” refers to a variant of a polypeptide or protein having substantial or significant sequence identity to the polypeptide or protein and retaining at least one of the biological activities of the polypeptide or protein. A functional variant of a polypeptide or protein can be prepared by means known in the art in view of the present disclosure. A functional variant can include one or more modifications to the amino acid sequence of the polypeptide or protein. In some embodiments, the modifications change one or more physicochemical properties of the polypeptide or protein, for example, by improving the thermal stability of the polypeptide or protein, altering the substrate specificity, changing the optimal pH, reduce immunogenicity, and the like. In some embodiments, the modifications alter one or more of the biological activities of the polypeptide or protein, so long as they do not destroy or abolish all of the biological activities of the polypeptide or protein.

According to some embodiments of the invention, a functional variant of a polypeptide or protein comprises one or more of an amino acid substitution, preferably a conservative amino acid substitution, to the polypeptide or protein that does not significantly affect the biological activity of the polypeptide or protein. Conservative amino acid substitutions include, but are not limited to, amino acid substitutions within the group of basic amino acids (arginine, lysine, and histidine), amino acid substitutions with the group of acidic amino acids (glutamic acid and aspartic acid), amino acid substitutions within the group of polar amino acids (glutamine and asparagine), amino acid substitutions within the group of hydrophobic amino acids (leucine, isoleucine, and valine), amino acid substitutions within the group of aromatic amino acids (phenylalanine, tryptophan, and tyrosine), and amino acid substitutions within the group of small amino acids (glycine, alanine, serine, threonine, and methionine). Non-standard or unnatural amino acids (such as 4-hydroxyproline, 6-N-methyl lysine, 2-aminoisobutyric acid, isovaline, and alpha-methyl serine) may also or alternatively be used to substitute standard amino acid residues in a polypeptide or protein.

According to some embodiments of the invention, a functional variant of a polypeptide or protein comprises a deletion and/or insertion of one or more amino acids to the polypeptide or protein. For example, a functional variant of CD can include a deletion and/or insertion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids to CD. For example, a functional variant of a sdAb can include a deletion and/or insertion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids to the sdAb. In some embodiments, a functional variant of a sdAb can include a deletion and/or insertion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids to the framework (FR) region of the sdAb. In some embodiments, the functional variant of a polypeptide or protein comprises a deletion of the first methionine.

According to some embodiments of the invention, a functional variant of a polypeptide or protein comprises a substitution and a deletion and/or insertion to the parent protein. In some aspects, the substitution is a conservative substitution and/or the deletion and/or insertion is a small deletion and/or small insertion.

As used herein, “single domain antibody,” “sdAb,” “sdAb protein,” “VHH” (variable domain of heavy chain antibody), and “nanobody” are used interchangeably. As used herein, “CD,” “CDase,” and “CD protein” are used interchangeably.

The terms “identical” or percent “identity,” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The term “substantially identical” refers to two or more sequences or subsequences that have a specified percentage of amino acid residues or nucleotides that are the same (i.e., at least about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. The definition includes sequences that have deletions and/or additions, as well as those that have substitutions. As described below, algorithms can account for gaps and the like. When not specified, identity or substantial identity is determined over the entire length of the reference sequence. When specified, identity can be determined over a region that is at least about 10 amino acids or nucleotides in length, at least about 25 amino acids or nucleotides in length, or over a region that is 50-100 amino acids or nucleotides in length.

The percent identity can be measured using sequence comparison software or algorithms or by visual inspection. Various algorithms and software are known in the art that can be used to obtain alignments of amino acid or nucleotide sequences (see e.g., Karlin et al., 1990, Proc. Natl. Acad. Sci. USA, 87:2264-2268, as modified in Karlin et al., 1993, Proc. Natl. Acad. Sci. USA, 90:5873-5877), and incorporated into the NBLAST and XBLAST programs (Altschul et al., 1991, Nucleic Acids Res., 25:3389-3402). In certain embodiments, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, Calif.), or MegAlign (DNASTAR).

The term “isolated,” when used to describe a protein or nucleic acid, refers to a molecule that is substantially free of other elements present in its natural environment. For instance, an isolated protein is substantially free of cellular material or other proteins from the cell or tissue source from which it is derived. The term “isolated” also refers to preparations where the isolated protein is sufficiently pure to be administered as a pharmaceutical composition, or at least 70-80% (w/w) pure, more preferably, at least 80-90% (w/w) pure, even more preferably, 90-95% pure; and, most preferably, at least 95%, 96%, 97%, 98%, 99%, or 100% (w/w) pure.

As used herein, “reference” in the context of comparison data refers to a standard of comparison.

As used herein, “specifically binds” refers to an agent (e.g., sdAb or functional variant thereof) that recognizes and binds a molecule (e.g., VEGFR2, EGFR), but which does not substantially recognize and bind other molecules in a sample, for example, other molecules in a biological sample. For example, two molecules that specifically bind form a complex that is relatively stable under physiologic conditions. Specific binding is characterized by a high affinity and a low-to-moderate number of binding sites, as distinguished from nonspecific binding, which usually has a low affinity with a moderate-to-high number of binding sites. As used herein, the term “specifically binds to” or is “specific for” refers to measurable and reproducible interactions such as binding between a target and an antibody (e.g., sdAb or functional variant thereof), which is determinative of the presence of the target in the presence of a heterogeneous population of molecules inducing biological molecules. For example, a sdAb or functional variant thereof that specifically binds to a target (which can be an epitope) is a sdAb or functional variant thereof that binds this target with greater affinity, avidity, more readily, and/or with greater duration than it binds to other targets. In some embodiments, the extent of binding of a sdAb or functional variant thereof to an unrelated target is less than about 10% of the binding of the sdAb or functional variant thereof to the target as measured, e.g., by a radioimmunoassay (RIA). In certain embodiments, a sdAb or functional variant thereof that specifically binds to a target has a dissociation constant of (K_{D}) of <1×10⁻⁶ M, <1×10⁻⁷ M, <1×10⁻⁸ M, <1×10⁻⁹M, or <1×10⁻¹⁰ M, <1×10⁻¹¹M, <1×10⁻¹² M. In certain embodiments, a sdAb or functional variant thereof specifically binds to an epitope on a protein that is conserved among the protein from different species. In some embodiments, specific binding can include, but does not require, exclusive binding (i.e., it can bind to only one protein).

As used herein, “subject” refers to a mammal, including, but not limited to, a human or non-human mammal, such as a bovine, equine, canine, ovine, or feline.

Ranges provided herein are understood to be shorthand for and thus encompass all of the values within the range. For example, a range of 1 to 50 is understood to include any number, combination of numbers, or sub-range chosen from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, including non-integers thereof.

The term “tumor” or “neoplasm” refers to an abnormal mass of tissue containing neoplastic cells. Neoplasms and tumors may be benign, premalignant, or malignant.

The term “cancer” or “malignant neoplasm” refers to a cell that displays uncontrolled growth, invasion upon adjacent tissues, and often metastasis to other locations of the body. This includes hematologic and lymphoid cancers.

The term “binding affinity” generally refers to the strength of the sum total of non-covalent interactions between a single binding site of a molecule (e.g., of a sdAb) and its binding partner (e.g., an antigen). Unless indicated otherwise, as used herein, “binding affinity,” “bind to,” “binds to,” or “binding to” refers to an intrinsic binding affinity that reflects a 1:1 interaction between members of a binding pair (e.g., antibody Fab fragment and antigen). The affinity of a molecule X for its partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative and exemplary embodiments for measuring binding affinity, e.g., binding strength are described in the following.

Various publications, articles, and patents are cited or described in the background and throughout the specification; each of these references is herein incorporated by reference in its entirety. Discussion of documents, acts, materials, devices, articles, or the like which have been included in the present specification is for the purpose of providing context for the invention. Such discussion is not an admission that any or all of these matters form part of the prior art with respect to any inventions disclosed or claimed.

The disclosure provides fusion proteins comprising formula (I) or formula (II):

N-(L)n-C (formula I);

C-(L)n-N (formula II);

wherein N is a single-domain antibody (sdAb) or functional variant thereof, L is a peptide linker and n=0-50, and C is a cytosine deaminase (CD) protein or a functional variant thereof. In some embodiments, the fusion protein consists essentially of formula I. In some embodiments, the fusion protein consists of formula I. In some embodiments, the fusion protein consists essentially of formula II. In some embodiments, the fusion protein consists of formula II.

In some embodiments, the fusion protein comprises formula I such that the C-terminus of the peptide linker or the C-terminus of the sdAb or functional variant thereof is fused to the N-terminus of the CD protein or functional variant thereof. For example, in some aspects, the peptide linker is not present and the C-terminus of the sdAb or functional variant thereof is fused to the N-terminus of the CD protein or functional variant thereof. In some aspects, the peptide linker is present and the C-terminus of the sdAb or functional variant thereof is fused to the N-terminus of the peptide linker, and the C-terminus of the peptide linker is fused to the N-terminus of the CD protein or functional variant thereof.

In other embodiments, the fusion protein comprises formula II such that the C-terminus of the peptide linker or the C-terminus of the CD protein or functional variant thereof is fused to the N-terminus of the sdAb or functional variant thereof. For example, in some aspects, the peptide linker is not present and the C-terminus of the CD protein or functional variant thereof is fused to the N-terminus of the sdAb or functional variant thereof. In some aspects, the peptide linker is present and the C-terminus of the CD protein or functional variant thereof is fused to the N-terminus of the peptide linker, and the C-terminus of the peptide linker is fused to the N-terminus of the sdAb or functional variant thereof.

### Exemplary Single-Domain Antibodies of the Fusion Proteins of the Disclosure

A sdAb comprises a single chain with a single variable domain having three complementarity determining regions (CDRs). Single-domain antibodies traditionally comprise the variable fragments of Camelid heavy-chain only antibodies (HcAbs), i.e., the variable domain of the heavy-chain of heavy-chain antibodies, VHH. Some VHH polypeptides are also referred to as the trademarked name Nanobody® (Nb;Ablynx). However, in this disclosure, the use of the term nanobody does not limit the single-domain antibody to a Nanobody® provided by Ablynx. In some embodiments, the sdAb or functional variant thereof is selected from heavy chain only IgG class antibodies of camels, alpacas, or llamas. In some embodiments, the sdAb or functional variant thereof is an engineered polypeptide generated by CDR grafting. For example, the sdAb or functional variant thereof is generated by replacing all or some of the CDR region of the camelid derived sdAbs with the CDR region of other known antibodies having desired targets. In some embodiments, the sdAb or functional variant thereof is a recombinant derivative of heavy-chain-only antibodies found in sharks. In some embodiments, the sdAb or functional variant thereof is synthetically generated using techniques that are well known in the art. In certain embodiments, a sdAb or functional variant thereof is a human sdAb (Domantis, Inc., Waltham, Mass.; see, e.g., U.S. Pat. No. 6,248,516 B1).

In some embodiments, the sdAb or functional variant thereof is a humanized VHH from a Camelid. As used herein, the term “humanized” sdAb means a sdAb that has had one or more amino acid residues in the amino acid sequence of the naturally occurring VHH sequence replaced by one or more amino acid residues that occur at the corresponding position in a VH domain from a conventional four-chain antibody from a human. This substitution can be performed by methods that are well known in the art. For example, the framework regions (FRs) of the sdAbs can be replaced by human variable FRs.

The CDRs are of primary importance for epitope recognition of a sdAb or functional variant thereof. Changes may be made to the amino acid residues that make up the CDRs without interfering with the ability of the sdAb or functional variant thereof to recognize and bind its cognate epitope. For example, changes that do not affect target epitope recognition, yet increase the binding affinity of the sdAb or functional variant thereof for the epitope, may be made. In some embodiments, these changes are conservative amino acid substitutions and/or small deletions or small insertions.

In some embodiments, the sdAbs or functional variants thereof comprise the variable domain of any one of a heavy chain-only IgG class of antibodies. The single variable domain comprises three complementarity-determining regions (CDRs). A sdAb or functional variant thereof can be an immunoglobulin and/or polypeptide with the (general) structure FR1-CDR1-FR2-CDR2-FR3-CDR3-FR4, in which FR1, FR2, FR3, and FR4 refer to framework regions 1, 2, 3, and 4, respectively, and in which CDR1, CDR2, and CDR3 refer to complementarity determining regions 1, 2, and 3, respectively. The numbering of the amino acid residues of a sdAb or functional variant thereof is according to the general numbering for VH domains given by Kabat et al. (“Sequence of proteins of immunological interest,” US Public Health Services, NIH Bethesda, Md., Publication No. 91). According to this numbering, FR1 of a sdAb comprises the amino acid residues at positions 1-30, complementarity-determining region 1 (CDR1) of a sdAb or functional variant thereof comprises the amino acid residues at positions 31-35, FR2 of a sdAb or functional variant thereof comprises the amino acids at positions 36-49, CDR2 of a sdAb or functional variant thereof comprises the amino acid residues at positions 50-65, FR3 of a sdAb or functional variant thereof comprises the amino acid residues at positions 66-94, CDR3 of a sdAb or functional variant thereof comprises the amino acid residues at positions 95-102, and FR4 of a sdAb or functional variant thereof comprises the amino acid residues at positions 103-113.

In some embodiments, the sdAb or functional variant thereof is a half-life extended sdAb. Methods of increasing the half-life of polypeptides are well known in the art.

The effectiveness of the fusion proteins of the present disclosure as therapeutic agents depends on the selection of an appropriate sdAb target. The target may be of any kind presently known, or that becomes known, and includes peptides and non-peptides (e.g., cell surface lipids, glycan or other post-translational modifications).

In some embodiments, the sdAbs or functional variants thereof of the disclosure bind to extracellular targets on the surface of cancer cells. In some embodiments, the sdAb or functional variant thereof binds to a target, wherein the target is selected from a cell membrane molecule, a secreted molecule, and an intracellular molecule.

In some embodiments, the target is selected from the group consisting of EGFR, 5T4, A33, AFP, Beta-catenin, BRCA1, BRCA2, C242, CCR4, CD152, CD19, CD20, CD200, CD22, CD221, CD23, CD30, CD3, CD37, CD40, CD44, CD5, CD51, CD52, CD56, CD64, CD74, CD80, CDCP1, c-KIT, COX-2, cMET, CSF1R, CTLA-4, EGF2, ErbB2, ErbB3, FGFR1, FGFR2, FGFR3, FLT3, HER2, HER3, HIF-Ia, HLA-DR, IGF-IR, mTOR, NPC-1C, P53, PDGFRα, PDGFRβ, PLGF, PSA, RGMa, RoN, TNF, TP53, TPD52, VEGFR1, VEGFR2, VEGFR3, CA-IX, αvβ3, α5β1, FAP, glycoprotein 75, TAG-72, MUC16, NR-LU-13, SLAMF7, EGP40, BAFF, PRL-3, carcinoembryonic antigen (CEA), prostate-specific membrane antigen, MART-1, gp100, Cancer-testis (CT) antigens (e.g. NY-ESO-1, MAGE-A3, MAGE-A1), hTERT, MCC, Mum-1, ERBB2IP, EpCAM, TfR, integrin α6β4, HGFR, PTP-LAR, CD147, CDCP1, CEACAM6, JAM1, integrin α3β1, integrin αvβ3, PD-L1, AXL, CDH6, DLL3, EDNRB, EFNA4, NEPP3, EPHA2, FOLR1, LewisY, GPNMB, GUCY2C, HAVCR1, Integrin α, LYPD3, Mesothelin, MUC1, NECTIN4, NOTCH3, PTK7, SLC34A2, SLC39A6, SLC44A4, SLITRK6, STEAP1, TACSTD2, TPBG, TIM-1, GD2, and nicotinic acetylcholine receptor (nAChR).

In some embodiments, the target is selected from the group consisting of EGFR, c-KIT, cMET, HER2, HER3, FGFR1, FGFR2, FGFR3, IGF-IR, P53, PDGFRα, VEGFR1, VEGFR2, VEGFR3, CA-IX, αvβ3, α5β1, MUC16, carcinoembryonic antigen (CEA), prostate-specific membrane antigen, Cancer-testis (CT) antigens (e.g., NY-ESO-1, MAGE-A3, MAGE-A1), Mesothelin, EpCAM, integrin α6β4, CEACAM6, integrin α3β1, integrin αvβ3, PD-L1, AXL, CDH6, DLL3, EDNRB, EFNA4, NEPP3, EPHA2, FOLR1, LewisY, GPNMB, GUCY2C, HAVCR1, Integrin α, LYPD3, MUC1, NECTIN4, NOTCH3, PTK7, SLC34A2, SLC39A6, SLC44A4, SLITRK6, STEAP1, TACSTD2, TPBG, TIM-1, GD2, and nicotinic acetylcholine receptor (nAChR).

In certain embodiments, the target is VEGFR2, EGFR, CEA, HER2, or HER3.

In certain embodiments, the sdAb or functional variant thereof of a fusion protein of the disclosure targets EGFR. In some embodiments, the sdAb or functional variant thereof is derived from VHH122, described in J. Biomol. Screen. 2009 January; 14(1):77-85. In some embodiments, the sdAb or functional variant thereof is derived from 7D12, described as Structure 21, 1214-1224, Jul. 2, 2013. PDB: 4KRM_I, and U.S. Patent Publication No. US2009/0252681.

In certain embodiments, the sdAb or functional variant thereof of a fusion protein of the disclosure targets VEGFR2. In some embodiments, the sdAb or functional variant thereof is derived from 3VGR19, described in Mol. Immunol. 2012 February; 50(1-2):35-41. In some embodiments, the sdAb or functional variant thereof is derived from 4VGR17, described in Mol. Immunol. 2012 February; 50(1-2):35-41. In some embodiments, the sdAb or functional variant thereof is derived from 4VGR38, described in Mol. Immunol. 2012 February; 50(1-2):35-41.

In certain embodiments, the sdAb or functional variant thereof of a fusion protein of the disclosure targets HER2. In some embodiments, the sdAb or functional variant thereof is derived from 2D3, 5F7, or 47D5, described in U.S. Patent Publication US 2011/0059090.

In certain embodiments, the sdAb or functional variant thereof of a fusion protein of the disclosure targets HER3. In some embodiments, the sdAb or functional variant thereof is derived from BCD090-M2 (PDB ID: 6EZW), described in Version 2. F1000Res. 2018; 7: 57.

In certain embodiments, the sdAb or functional variant thereof of a fusion protein of the disclosure binds CEA. In some embodiments, the sdAb or functional variant thereof is derived from ABS29544.1 or NbCEA5, described in US20160280795A1, FEBS J. 2009 July; 276(14):3881-93 and J Nucl Med. 2010 July; 51(7):1099-106.

The biological activity of a sdAb or functional variant thereof of a fusion protein of the disclosure can be assessed by, for example, determining its binding affinity to a target or an epitope thereof. In some embodiments, the affinity of the sdAb or functional variant thereof of the fusion protein for a target or an epitope thereof, can be, for example, from about 1 picomolar (pM) to about 100 micromolar (μM) (e.g., from about 1 picomolar (pM) to about 1 nanomolar (nM), from about 1 nM to about 1 micromolar (μM), or from about 1 μM to about 100 μM). In some embodiments, the sdAb or functional variant thereof of the fusion protein can bind to a target (e.g., VEGFR2, EGFR, HER2, HER3, or CEA) with a K_{D} less than or equal to 1 nanomolar (e.g., 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In some embodiments, the sdAb or functional variant thereof of the fusion protein of the disclosure can bind to the target with a K_{D} less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values). The affinity of a sdAb or functional variant thereof of a fusion protein disclosed herein for an antigen or epitope of interest can be measured using any art-recognized assay. Such methods include, for example, fluorescence activated cell sorting (FACS), separable beads (e.g., magnetic beads), surface plasmon resonance (SPR), solution phase competition (KINEXA™), antigen panning, and/or ELISA (see, e.g., Janeway et al. (eds.), Immunobiology, 5th ed., Garland Publishing, New York, N.Y., 2001).

The sdAbs (or functional variants thereof) and nucleic acids used to make the fusion proteins of the disclosure can be prepared as described by the below Examples or by any method known to one of ordinary skill in the art.

In some embodiments, the sdAb or functional variant thereof comprises, consists essentially of, or consists of a CDR1 selected from the group consisting of SEQ ID NOs: 28, 31, and 34; a CDR2 selected from the group consisting of SEQ ID NOs: 29, 32, and 35; and a CDR3 selected from the group consisting of SEQ ID NOs: 30, 33, and 36.

In some embodiments, the sdAb or functional variant thereof comprises, consists essentially of, or consists of a CDR1 selected from the group consisting of SEQ ID NOs: 37 and 40, a CDR2 selected from the group consisting of SEQ ID NOs: 38 and 41, and a CDR3 selected from the group consisting of SEQ ID NOs: 39 and 42.

In some embodiments, the sdAb or functional variant thereof comprises, consists essentially of, or consists of a CDR1 selected from the group consisting of SEQ ID NOs: 199, 202, and 205; a CDR2 selected from the group consisting of SEQ ID NOs: 200, 203, and 206; and a CDR3 selected from the group consisting of SEQ ID NOs: 201, 204, and 207.

In some embodiments, the sdAb or functional variant thereof comprises, consists essentially of, or consists of a CDR1 of SEQ ID NO: 208, a CDR2 of SEQ ID NO: 209, and a CDR3 of SEQ ID NO: 210.

In some embodiments, the sdAb or functional variant thereof comprises, consists essentially of, or consists of a CDR1 selected from the group consisting of SEQ ID NOs: 211 and 214, a CDR2 selected from the group consisting of SEQ ID NOs: 212 and 215, and a CDR3 selected from the group consisting of SEQ ID NOs: 213 and 216.

In some embodiments, the sdAb or functional variant thereof is selected from the group of anti-VEGFR2 sdAbs disclosed in the Examples, namely 3VGR19 (SEQ ID NO: 23), 4VGR17 (SEQ ID NO: 24), and 4VGR38 (SEQ ID NO: 25). In some embodiments, the sdAb or functional variant thereof is selected from the group of anti-EGFR sdAbs disclosed in the Examples, namely VHH122 (SEQ ID NO: 26) and 7D12 (SEQ ID NO: 27). In some embodiments, the sdAb or functional variant thereof is selected from the group of anti-HER2 sdAbs disclosed in the Examples, namely 2D3 (SEQ ID NO: 69), 5F7 (SEQ ID NO: 70) and 47D5 (SEQ ID NO: 71). In some embodiments, the sdAb or functional variant thereof is selected from the group of anti-HER3 sdAbs disclosed in the Examples, namely BCD090-M2 (SEQ ID NO: 75). In some embodiments, the sdAb or functional variant thereof is selected from the anti-CEA sdAbs disclosed in the Examples, namely anti-CEA sdAb (ABS29544.1) (SEQ ID NO: 77) and NbCEA5 (SEQ ID NO: 79).

In some embodiments, any of the sdAbs or functional variants thereof disclosed herein are fused with a yeast CD protein or functional variant thereof comprising the amino acid sequence of SEQ ID NO: 21 (wild type CD, without the N-terminal methionine), SEQ ID NO: 22 (a variant of SEQ ID NO: 21 with point mutations A22L/V107I/I139L), SEQ ID NO: 186 (wild type CD, including the N-terminal methionine), SEQ ID NO: 187 (a variant of SEQ ID NO: 186 with point mutations A23L/V108I/I140L), or a functional variant of any of the foregoing amino acid sequences.

In certain embodiments, the fusion proteins comprise a histidine tag (HIS-tag) at the C-terminus, e.g., a 6-HIS tag (SEQ ID NO: 6). In some embodiments, the fusion protein is linked to the HIS-tag via a peptide linker (e.g., GSS).

In some embodiments, the sdAb or functional variant thereof of a fusion protein of the disclosure can be partially or fully humanized for use in prophylaxis and/or therapy of a condition that is positively associated with the presence of the sdAb's target. In general, humanization involves replacing all or some of the camelid derived framework and variable regions of a sdAb or functional variant thereof with a human counterpart sequence, with the aim being to reduce immunogenicity of the sdAb in therapeutic applications. In some instances, the FR residues of the camelid immunoglobulin are replaced by corresponding human residues.

In certain embodiments, the sdAb or functional variant thereof comprises one or more mutations on the following T cell epitopes:

| Epitope | HLA-DR restricted epitope | SEQ ID NO |
|---|---|---|
| Epitope 1 | SVQTGGSLRL | 63 |
| Epitope 2 | LKPEDTAIY | 64 |
| Epitope 3 | IYYCAAAAGS | 65 |

In certain embodiments, the sdAb or functional variant thereof comprises a sequence X₁X₂QX₃X₄GX₅LRL modified from Epitope 1 (SEQ ID NO: 63), wherein X₁ can be substituted by V, X₂ can be substituted by A, X₃ can be substituted by V, X4 can be substituted by D, and/or X5 can be substituted by D or E. In certain embodiments, the sdAb or functional variant thereof comprises a sequence LX₁X₂EDX₃X₄X₅Y modified from Epitope 2 (SEQ ID NO: 64), wherein X₁ can be substituted by R, A, D, S, or T; X2 can be substituted by A, D, or E; X3 can be substituted by D, E, G, H, or Q; X4 can be substituted by D or E; and/or X5 can be substituted by V, A, T, R, Q, or N. In certain embodiments, the sdAb or functional variant thereof comprises a sequence X₁YYCAAAAGS modified from Epitope 3 (SEQ ID NO: 65), wherein X₁ can be substituted by V, A, T, R, Q, or N.

In certain embodiments, the sdAb or functional variant thereof comprises a sequence X₁X₂QX₃X₄GSLRL modified from Epitope 1 (SEQ ID NO: 63), wherein X₁ can be substituted by V, X₂ can be substituted by A, X₃ can be substituted by V, and/or X₄ can be substituted by D. In certain embodiments, the sdAb or functional variant thereof comprises a sequence LXiX₂EDTAX₅Y modified from Epitope 2 (SEQ ID NO: 64), wherein X₁ can be substituted by R or T; X₂ can be substituted by A; and/or X₅ can be substituted by V or R. In certain embodiments, the sdAb or functional variant thereof comprises a sequence X₁YYCAAAAGS modified from Epitope 3 (SEQ ID NO: 65), wherein X₁ can be substituted by V or R.

In certain embodiments, the sdAb or functional variant thereof comprises a FR1 sequence with a sequence ESGGGX₁X₂QX₃GGSL, wherein X₁ is S or V, X₂ is V or A, and X₃ is A, T, or V. In certain embodiments, the sdAb or functional variant thereof comprises a FR3 sequence comprising a modified sequence MNSLX₁X₂EDTAX₃YYCAA, wherein X₁ is K, R, or T; X₂ is P or A; and X₃ is R or V.

Single-domain antibody amino acid sequences are closely related to the human family III VH amino acid sequences. Accordingly, in some embodiments, “humanized” sdAb forms are produced. In some embodiments, sdAbs or functional variant thereof are derived from HCAb produced by immunizing a transgenic mouse with a target peptide in which endogenous murine antibody expression has been eliminated and human transgenes have been introduced. HCAb mice are disclosed in U.S. Pat. Nos. 8,883,150, 8,921,524, 8,921,522, 8,507,748, 8,502,014, US 2014/0356908, US2014/0033335, US2014/0037616, US2014/0356908, US2013/0344057, US2013/0323235, US2011/0118444, and US2009/0307787, all of which are incorporated herein by reference for all they disclose regarding heavy chain only antibodies and their production in transgenic mice. The HCAb mice are immunized and the resulting primed spleen cells are fused with murine myeloma cells to form hybridomas. The resultant HCAb can then be made fully human by replacing the murine CH2 and CH3 regions with corresponding human sequences.

Additional methods for making sdAbs or functional variants thereof for the fusion proteins of the disclosure are well known in the art. For example, one method for obtaining sdAbs or functional variants thereof includes (a) immunizing a Camelid with one or more antigens, (b) isolating peripheral lymphocytes from the immunized Camelid, obtaining the total RNA and synthesizing the corresponding cDNAs, (c) constructing a library of cDNA fragments encoding sdAb domains, (d) transcribing the sdAb domain-encoding cDNAs obtained in step (c) to mRNA using PCR, converting the mRNA to ribosome or phage display format, and selecting the sdAb domain by ribosome display or phage display panning, and (e) expressing the sdAb domain in a suitable vector and, optionally, purifying the expressed sdAb domain. Other exemplary methods are described in any one of the references provided in Revets et al, 2015 Expert Opin. Biol. Ther. (2005) 5(1):111-124, “Generation and production of recombinant nanobodies.” In addition, Harbour Biomed provides a platform of human heavy chain rodent technology and human heavy chain constructs, described in U.S. Pat. Nos. 9,353,179, 9,346,877, and 8,921,522, and European Patents 1776383 and 1864998, all of which are incorporated herein by reference. Ablynx also provides a source of nanobodies that can be used to make the compounds of the disclosure.

For a further description of nanobodies, reference is made to the following references, each of which is incorporated herein by reference: review article by Muyldermans (Reviews in Molecular Biotechnology 74: 277-302, 2001), as well as to the following patent applications, which are mentioned as general background art: WO 94/04678, WO 95/04079, and WO 96/34103 of the Vrije Universiteit Brussel; WO 94/25591, WO 99/37681, WO 00/40968, WO 00/43507, WO 00/65057, WO 01/40310, WO 01/44301, EP 1134231, and WO 02/48193 of Unilever; WO 97/49805, WO 01/21817, WO 03/035694, WO 03/054016, and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (corresponding to EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787, and WO 06/122825, by Ablynx N.V. and the further published patent applications by Ablynx N.V. Reference is also made to the further art mentioned in these applications, and in particular to the list of references mentioned on pages 41-43 of the International application WO 06/040153, which list and references are incorporated herein by reference. As described in these references, nanobodies (in particular, VHH sequences and partially humanized nanobodies) can in particular be characterized by the presence of one or more “Hallmark residues” in one or more of the framework sequences. A further description of the nanobodies, including humanization and/or camelization of nanobodies, as well as other modifications, parts or fragments, derivatives or “nanobody fusions,” multivalent constructs (including some non-limiting examples of linker sequences) and different modifications to increase the half-life of the nanobodies and their preparations can be found, e.g., in WO 08/101985 and WO 08/142164. For a further general description of nanobodies, reference is made WO 08/020079 (page 16, page 61, line 24 to page 98, line 3).

### Exemplary Linkers of the Fusion Proteins of the Disclosure

The terms “linker,” “peptide linker,” “linker domain,” and “linker region” (abbreviated “L”) as used herein are used interchangeably and refer to an oligo- or polypeptide region from about 1 to 100 amino acids in length, which links together polypeptides of the fusion proteins of the disclosure (e.g., the sdAb or functional variant thereof and the CD protein or functional variant thereof). A fusion protein of the disclosure may comprise one or more than one linker. Linkers may be composed of flexible residues like glycine and serine so that the adjacent protein domains are free to move relative to one another. In some embodiments, the linker comprises 1 to 20, 1 to 15, 1 to 10, or 1 to nine amino acids. In some embodiments, the linker comprises from one to nine amino acids, e.g., one, two, three, four, five, six, seven, eight, or nine amino acids. In some embodiments, the linker is a peptide that ranges from about 6 to about 30 amino acids in length. In aspects of these embodiments, the peptide linker can be, e.g., at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29 or 30 amino acids in length. In other aspects of these embodiments, the peptide linker can be, e.g., at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, or at most 30 amino acids in length. In other aspects of these embodiments, the peptide linker can be, e.g., about 6 to about 8, about 6 to about 10, about 6 to about 12, about 6 to about 14, about 6 to about 16, about 6 to about 18, about 6 to about 20, about 6 to about 22, about 6 to about 24, about 6 to about 26, about 6 to about 28, about 6 to about 30, about 8 to about 10, about 8 to about 12, about 8 to about 14, about 8 to about 16, about 8 to about 18, about 8 to about 20, about 8 to about 22, about 8 to about 24, about 8 to about 26, about 8 to about 28, about 8 to about 30, about 10 to about 12, about 10 to about 14, about 10 to about 16, about 10 to about 18, about 10 to about 20, about 10 to about 22, about 10 to about 24, about 10 to about 26, about 10 to about 28, about 10 to about 30, about 12 to about 14, about 12 to about 16, about 12 to about 18, about 12 to about 20, about 12 to about 22, about 12 to about 24, about 12 to about 26, about 12 to about 28, about 12 to about 30, about 14 to about 16, about 14 to about 18, about 14 to about 20, about 14 to about 22, about 14 to about 24, about 14 to about 26, about 14 to about 28, about 14 to about 30, about 16 to about 18, about 16 to about 20, about 16 to about 22, about 16 to about 24, about 16 to about 26, about 16 to about 28, about 16 to about 30, about 18 to about 20, about 18 to about 22, about 18 to about 24, about 18 to about 26, about 18 to about 28, about 18 to about 30, about 20 to about 22, about 20 to about 24, about 20 to about 26, about 20 to about 28, about 20 to about 30, about 22 to about 24, about 22 to about 26, about 22 to about 28, about 22 to about 30, about 24 to about 26, about 24 to about 28, about 24 to about 30, about 26 to about 28, about 26 to about 30, or about 26 to about 30 amino acids in length. In some embodiments, the linker region comprises 1-50 amino acids.

The linker can comprise natural or non-natural amino acids. In some embodiments, the linker comprises any amino acid, combinations of different amino acids, or the same amino acid. The linkers can be peptide sequences of different kinds linked together, one or more times in a row, or alternating with other sequences. The linkers can also be peptide sequences that are repeated. In some embodiments, an entire peptide sequence is repeated 1 to 50 times. Longer linkers may also be used when it is desirable to ensure that two adjacent domains do not sterically interfere with one another.

In some embodiments, the linker is (GGGGS)n, where n=1, 2, 3, 4, 5 or 6 (SEQ ID NO: 1). In some embodiments, the linker is one or more of GSGG (SEQ ID NO: 2), GGGGSGGGS (SEQ ID NO: 3), and/or one or more GSG. In some embodiments, the linker is KESGSVSSEQLAQFRSLD (SEQ ID NO: 4) or EGKSSGSGSESKST (SEQ ID NO: 5). In some embodiments, the linker is (GGGGS)3 (SEQ ID NO: 188). Exemplary amino acid residues for use in linkers include glycine, threonine, arginine, serine, alanine, asparagine, glutamine, aspartic acid, proline, glutamic acid, and/or lysine. Examples of other linkers that can be used for fusion proteins are well known in the art and some can be found in Chen, X. et al., Adv Drug Deliv. Rev. 2013 Oct. 15; 65(10): 1357-1369, incorporated herein by reference in its entirety. Additional linkers suitable for fusion proteins of the present disclosure can also be found in Klein et al., Protein Eng. Des. Sel. 2014 October; 27(10): 325-330, incorporated herein by reference in its entirety.

In some embodiments, a fusion protein of the disclosure comprises one linker between a sdAb or functional variant thereof and a CD protein or functional variant thereof. In some embodiments, the fusion protein comprises at least one linker between a sdAb or functional variant thereof and a CD protein or functional variant thereof. For example, in some aspects, the fusion protein comprises two linkers between a sdAb or functional variant thereof and a CD protein or functional variant thereof.

In some embodiments, a sdAb or functional variant thereof and a CD protein or functional variant thereof are fused directly (e.g., having no connecting linker).

### Exemplary Cytosine Deaminase (CD) Proteins of the Fusion Proteins of the Disclosure

Cytosine deaminase (CD) is an enzyme that is able to convert the relatively harmless 5-fluorocytosine (5-FC) prodrug into 5-fluorouracil (5-FU), which is a cytotoxic compound, in particular when it is converted to 5-fluorouridine 5′-monophosphate (5-FdUMP). Accordingly, fusion proteins of the disclosure will bind to cells that express the target of the sdAbs or functional variants thereof of the disclosure, and the CD protein or functional variant thereof of the fusion protein will convert 5-FC into 5-FU, thereby killing the target cells. In some embodiments, the target cells are cancer cells. In some embodiments, the target cells are killed through a bystander effect. Anticancer Res. 1998 September-October; 18(5A):3399-406; Am J Cancer Res. 2015; 5(9): 2686-2696.

In some embodiments, the CD or functional variant thereof may be yeast CD or a functional variant thereof. In some embodiments, the CD or functional variant thereof may be a bacterial CD or a functional variant thereof. In some embodiments, the CD or functional variant thereof is E. coli cytosine deaminase or a functional variant thereof. For example, in some embodiments, the CD or functional variant thereof is an E. coli cytosine deaminase represented by NCBI Reference Sequence: NP_414871.1. In some embodiments, the CD or functional variant thereof is a yeast cytosine deaminase represented by GenBank Accession No. AAB67713.1. The FCY1 gene of Saccharomyces cerevisiae (S. cerevisiae) and the coda gene of E. coli, which encode, respectively, the CD of these two organisms, are known and their sequences are published (EP 402108; Erbs et al., 1997, Curr. Genet. 31, 1-6; WO 93/01281).

In certain embodiments, the CD protein or functional variant thereof comprises the sequence of SEQ ID NO: 21. In certain embodiments, the CD protein or functional variant thereof comprises the sequence of SEQ ID NO: 186. In certain embodiments, the sequence corresponding to cytosine deaminase contains one or more alterations. In some embodiments, the alterations result in a functional variant of a wild-type CD. Preferably, the alterations in the CD domain are stabilizing mutations. For example, in some embodiments, the functional variant of CD comprises the sequence of SEQ ID NO: 22, having A22L/V107I/I139L compared to SEQ ID NO: 21. In some embodiments, the functional variant of CD comprises the sequence of SEQ ID NO: 187, having A23L/V108I/I140L compared to SEQ ID NO: 186.

The disclosure also provides fusion proteins comprising a CD polypeptide that comprises a sequence that is at least 80%, 90%, 95%, 98% or 99% identical to SEQ ID NO: 21, 22, 186, or 187, wherein the polypeptide has cytosine deaminase activity and thus is called a “functional variant.”

In some embodiments, the functional variant of CD protein can be a de-immunized form to reduce immunogenicity. In certain embodiments, the CD protein or functional variant thereof comprises one or more mutations on the following T cell epitopes:

| Epitope | HLA-DR restricted epitope | SEQ ID NO |
|---|---|---|
| Epitope 4 | YTTLSPCDM | 66 |
| Epitope 5 | MCTGAIIMY | 67 |
| Epitope 6 | VVVVDDERCKK | 68 |

In certain embodiments, the CD protein or functional variant thereof comprises a sequence X₁X₂X₃LSPCD X₄ modified from Epitope 4 (SEQ ID NO: 66), wherein X₁ can be substituted by A or H, X₂ can be substituted by D, X₃ can be substituted by E, and X₄ can be substituted by N, A, K, or Q. In certain embodiments, the CD protein or functional variant thereof comprises a sequence X₁CTGAIIMY modified from Epitope 5 (SEQ ID NO: 67), wherein X₁ can be substituted by N, A, K, or Q. In certain embodiments, the CD protein or functional variant thereof comprises a sequence X₁X₂X₃VDDERCKK modified from Epitope 6 (SEQ ID NO: 68), wherein X₁ can be substituted by A or T, X₂ can be substituted by A, L, I, or T, and X₃ can be substituted by A or T.

In certain embodiments, the CD protein or functional variant thereof comprises a sequence X₁X₂VVDDERCKK modified from Epitope 6 (SEQ ID NO: 68), wherein X₁ can be substituted by A, X₂ can be substituted by T.

In some embodiments, CD functional variants are variants of SEQ ID NOs: 21 or 22, wherein the variants comprise at least one mutation selected from Y84A, Y84H, T85D, T86E, M92N, M92A, M92K, M92Q, V128A, V128T, V129A, V129L, V129I, V129T, V130A, and V130T. In some embodiments, CD functional variants are variants of SEQ ID NOs: 186 or 187, wherein the variants comprise at least one mutation selected from Y85A, Y85H, T86D, T87E, M93N, M93A, M93K, M93Q, V129A, V129T, V130A, V130L, V130I, V130T, V131A, and V131T.

In certain embodiments, the functional variant of the CD protein is selected from the group consisting of SEQ ID NOs: 22, 187, 189, 190, 191, 192, 193, and 194. In certain embodiments, the functional variant of the CD protein is selected from the group consisting of SEQ ID NOs: 22, 189, 191, and 193.

Assays for measuring cytosine deaminase activity are known in the art. For example, cytosine deaminase activity can be measured by determining the rate of conversion of 5-FC to 5-FU or cytosine to uracil. The detection of 5-FC, 5-FU, cytosine, and uracil can be performed by the methods described in the Examples section, by chromatography, and/or by other methods known in the art.

### Exemplary Fusion Proteins of the Disclosure

The disclosure provides examples of several fusion proteins, as described below:

The disclosure provides fusion proteins comprising formula (I) or formula (II):

N-(L)n-C (formula I);

C-(L)n-N (formula II);

wherein N is a single-domain antibody (sdAb) or functional variant thereof, L is a peptide linker and n=0-50, and C is a cytosine deaminase (CD) protein or a functional variant thereof. In some embodiments, the fusion protein consists essentially of formula I. In some embodiments, the fusion protein consists of formula I. In some embodiments, the fusion protein consists essentially of formula II. In some embodiments, the fusion protein consists of formula II.

In some embodiments, the fusion protein comprises formula I such that the C-terminus of the peptide linker or the C-terminus of the sdAb or functional variant thereof is fused to the N-terminus of the CD protein or functional variant thereof. For example, in some aspects, the peptide linker is not present and the C-terminus of the sdAb or functional variant thereof is fused to the N-terminus of the CD protein or functional variant thereof. In some aspects, the peptide linker is present and the C-terminus of the sdAb or functional variant thereof is fused to the N-terminus of the peptide linker, and the C-terminus of the peptide linker is fused to the N-terminus of the CD protein or functional variant thereof.

In other embodiments, the fusion protein comprises formula II such that the C-terminus of the peptide linker or the C-terminus of the CD protein or functional variant thereof is fused to the N-terminus of the sdAb or functional variant thereof. For example, in some aspects, the peptide linker is not present and the C-terminus of the CD protein or functional variant thereof is fused to the N-terminus of the sdAb or functional variant thereof. In some aspects, the peptide linker is present and the C-terminus of the CD protein or functional variant thereof is fused to the N-terminus of the peptide linker, and the C-terminus of the peptide linker is fused to the N-terminus of the sdAb or functional variant thereof.

The disclosure also provides fusion proteins comprising sdAbs—directed against extracellular target antigens, including, but not limited to, any of the target antigens described herein—fused to any of the cytosine deaminases described herein.

For example, in some embodiments, the fusion protein comprises the amino acid sequence of SEQ ID NOs: 7, 9, 11, 13, 15, 17, or amino acids 1-297 of any of the foregoing sequences. In some embodiments, the fusion protein consists essentially of the amino acid sequence of SEQ ID NOs: 7, 9, 11, 13, 15, 17, or amino acids 1-297 of any of the foregoing sequences. In some embodiments, the fusion protein consists of the amino acid sequence of SEQ ID NOs: 7, 9, 11, 13, 15, 17, or amino acids 1-297 of any of the foregoing sequences.

In some embodiments, the fusion protein comprises an amino acid sequence selected from the group consisting of SEQ ID NOs: 17, 19, and 93-185. In some embodiments, the fusion protein consists essentially of an amino acid sequence selected from the group consisting of SEQ ID NOs: 17, 19, and 93-185. In some embodiments, the fusion protein consists of an amino acid sequence selected from the group consisting of SEQ ID NOs: 17, 19, and 93-185.

In some embodiments, the fusion protein comprises amino acids 1-297 of an amino acid sequence selected from the group consisting of SEQ ID NOs: 93-181. In some embodiments, the fusion protein consists essentially of amino acids 1-297 of an amino acid sequence selected from the group consisting of SEQ ID NOs: 93-181. In some embodiments, the fusion protein consists of amino acids 1-297 of an amino acid sequence selected from the group consisting of SEQ ID NOs: 93-181.

In some embodiments, the fusion protein comprises an amino acid sequence selected from the group consisting of SEQ ID NO: 19, 182, 183, 184, and 185. In some embodiments, the fusion protein consists essentially of an amino acid sequence selected from the group consisting of SEQ ID NO: 19, 182, 183, 184, and 185. In some embodiments, the fusion protein consists of an amino acid sequence selected from the group consisting of SEQ ID NO: 19, 182, 183, 184, and 185.

The disclosure also provides for fusion proteins comprising more than one sdAb or functional variant thereof or CD protein or functional variant thereof. For example, the fusion protein may have the following formula III or IV:

N1-[(L1)ₙ-N2]ₙ₁-(L2)ₙ-(C)-[(L3)ₙ-C]ₙ₂ (formula III);

(C)-[(L1)ₙ-C]ₙ₂-(L2)ₙN1-[(L3)ₙ-N2]ₙ₁ (formula IV);

wherein: N1 and N2 are each a sdAb or a functional variant thereof, wherein N1 and N2 may be the same or different sdAb or functional variant thereof, and wherein n1=0-10; L1, L2, and L3 are each a peptide linker, wherein n=0-50; C is a cytosine deaminase (CD) protein or a functional variant thereof, wherein n2=0-10.

For example, the fusion protein may have the following formula:

N1-L1-N2-L2-C;

N1-L1-N2-L2-C-L3-C;

N1-N2-L-C;

N1-N2-L-C; or

N1-L1-C—N2-L2-C;

wherein N1 and N2 may be the same or different, and wherein any of L1, L2, and L3 may be the same or different.

In some embodiments, the fusion protein is a bivalent fusion protein comprising two different sdAbs or functional variants thereof (i.e., it binds two different targets or two different epitopes in the same target). In some embodiments, the fusion protein is a monovalent fusion protein.

The fusion proteins of the disclosure can be further fused with moieties, e.g., peptide tags, for ease in purification, see e.g., WO 93/21232; EP 439,095; Naramura et al., Immunol Lett 39:91 (1994); U.S. Pat. No. 5,474,981; Gillies et al., Proc Natl Acad Sci USA 89:1428 (1992); and Fell et al., J Immunol 146:2446 (1991). In some embodiments, the peptide tag is a histidine (HIS) tag. For example, in some embodiments, the peptide tag is a hexa-histidine peptide (SEQ ID NO: 6). The hexa-histidine tag may be the tag provided in a pQE vector (QIAGEN, Inc., Chatsworth, Calif.) or in another vector, many of which are commercially available, Gentz et al., Proc Natl Acad Sci USA 86:821 (1989). Other peptide tags useful for purification include, but are not limited to, the “HA” tag, which corresponds to an epitope derived from the influenza hemagglutinin protein (Wilson et al., Cell 37:767 (1984)) and the “FLAG™” tag. The peptide tag can be located at the N-terminus of the fusion protein, the C-terminus of the fusion protein, or in between functional domains (e.g., between sdAb and CD or functional variant(s) thereof) of the fusion protein. The peptide tag can be connected to the fusion protein by a peptide linker. For example, the peptide linker connecting the fusion protein and tag (e.g., HIS-tag) may be GSS.

The disclosure further encompasses fusion proteins that are conjugated to chemical moieties including, for example, cytotoxic/chemotherapeutic moieties and/or radiolabels. Any of the cytotoxic agents and chemotherapeutic agents described herein for combination treatment with the fusion proteins of the disclosure can be chemically conjugated to the fusion proteins of the disclosure. In some embodiments, the cytotoxic agent or chemotherapeutic agent is covalently conjugated to the fusion protein. In some embodiments, the cytotoxic agent of chemotherapeutic agent is non-covalently conjugated to the fusion protein.

In some embodiments, the conjugation of the fusion proteins of the disclosure to the cytotoxic agent or chemotherapeutic agent is through a linker selected from the group consisting of a disulfide group, a thioether group, an acid labile group, a photolabile group, a peptidase labile group, and an esterase labile group.

In some embodiments, the fusion proteins of the disclosure are conjugated to cytotoxic and/or cytostatic agents. In some aspects of these embodiments, the cytotoxic and/or cytostatic agent is conjugated to a sdAb or functional variant thereof of the fusion protein. In some aspects of these embodiments, the cytotoxic and/or cytostatic agent is conjugated to a CD protein or functional variant thereof of the fusion protein.

In other embodiments, the fusion protein is conjugated (chemically or genetically) or coupled to a cytokine, a superantigen, and/or a toxin.

In some embodiments, the pharmacokinetics of the fusion protein, including the half-life of the fusion protein, can be improved by chemical modification, such as the addition of poly(alkylene) glycol such as poly(ethylene) glycol (“PEGylation”), POLY PEG, PASylation, or by incorporation in a liposome. In some embodiments, the fusion protein (e.g., the sdAb or functional variant thereof) comprises one or more additional amino acid residues that allow for pegylation and/or facilitate pegylation (e.g., an additional cysteine residue for easy attachment of a PEG-group). In some embodiments, the half-life of the fusion protein is increased by attaching polysialic acid (PSA), hydroxyethyl starch (HES), an albumin-binding ligands or a carbohydrate shield to the fusion protein; by genetic fusion to a protein that binds serum proteins, such as albumin, IgG, FcRn, and/or transferrin; by genetic fusion to albumin or a domain of albumin, or to an albumin-binding protein; or by incorporation of the fusion protein into a nanocarrier, slow release formulation, or medical device.

In some embodiments the fusion protein can be modified by glycosylation, acetylation, phosphorylation, amidation, derivatization by known protecting/blocking groups, and/or proteolytic cleavage. These modifications may be carried out by known techniques, including, but not limited to, specific chemical cleavage, acetylation, formylation, and others known in the art. Additionally, the fusion protein may comprise one or more non-classical amino acids.

In some embodiments, such as for diagnostic or assay purposes (e.g., imaging to allow, for example, monitoring of therapies or tracking the distribution of the fusion protein), the fusion protein can comprise a detectable label. Suitable detectable labels and methods for labeling a protein are well known in the art. Suitable detectable labels include, for example, a radioisotope (e.g., Indium-111, Technetium-99m or Iodine-131), positron-emitting labels (e.g., Fluorine-19), paramagnetic ions (e.g., Gadolinium (III), Manganese (II)), an epitope label (tag), an affinity label (e.g., biotin, avidin), a spin label, an enzyme, a fluorescent group, or a chemiluminescent group. When labels are not employed, complex formation (e.g., between the fusion protein and a target) can be determined by surface plasmon resonance, ELISA, FACS, or other suitable methods known in the art.

### Nucleic Acids Encoding the Proteins of the Disclosure

Nucleic acids, including nucleotide sequences that encode the sdAbs, linkers, CD proteins, functional variants of sdAb and/or CD proteins, fusion proteins, or functional equivalents of any of the foregoing as described herein, are used in recombinant DNA molecules that direct the expression of the fusion proteins of the disclosure in appropriate host cells, such as bacterial cells. As used herein, the term “nucleic acid molecule” or “polynucleotide” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded.

In some aspects, the present disclosure pertains to polynucleotides that encode a cytosine deaminase or a mutant cytosine deaminase polypeptide or biologically active portions thereof; polynucleotides that encode a sdAb or biologically active variants thereof; polynucleotides that encode one or more linkers of the disclosure; and polynucleotides that encode a fusion protein of the disclosure.

In some embodiments, the polynucleotide encoding a CD or functional variant thereof is a codon-optimized polynucleotide. In some embodiments the CD polynucleotide or codon-optimized polynucleotide comprises recombinant, engineered, or isolated forms of naturally occurring nucleic acids isolated from an organism, e.g., a bacterial or yeast strain. Exemplary CD polynucleotides include those that encode a polypeptide set forth in SEQ ID NOs: 21, 22, 186, or 187. The nucleic acids used in the Examples of this disclosure are within the scope of the embodiments.

In some embodiments, CD or sdAb polynucleotides (including codon-optimized polynucleotides) are produced by diversifying, e.g., recombining and/or mutating one or more naturally occurring, isolated, or recombinant CD or sdAb polynucleotide sequences. As described in more detail elsewhere herein, it is possible to generate diverse CD or sdAb polynucleotides encoding CD or sdAb polypeptides (e.g., a functional variant of CD or sdAb) with superior functional attributes, e.g., increased catalytic function, increased stability, or higher expression level, than an unmodified CD or sdAb polynucleotide used as a substrate or parent in the diversification process. Due to the degeneracy of the genetic code, various nucleic acid sequences which encode substantially the same or a functionally equivalent amino acid sequence can be used to clone and/or express the fusion proteins of the disclosure.

The polynucleotides of the disclosure have a variety of uses in, for example, recombinant production (i.e., expression) of the fusion proteins of the disclosure and as substrates for further diversity generation, e.g., recombination reactions or mutation reactions to produce new and/or improved variants, and the like.

Certain specific, substantial, and credible utilities of the CD and single-domain antibody polynucleotides of the disclosure do not require that the polynucleotide encode a polypeptide with substantial CD activity, or even variant CD activity, or sdAb activity (e.g., target binding). For example, CD polynucleotides that do not encode active enzymes can be valuable sources of parental polynucleotides for use in diversification procedures to arrive at CD polynucleotide variants, or non-CD polynucleotides, with desirable functional properties (e.g., high kcat or kcat/Km, low Km, high stability towards heat or other environmental factors, high transcription or translation rates, resistance to proteolytic cleavage, increased antigen binding, increased antigen specificity, decreased immunogenicity).

In some embodiments, the polynucleotide encoding a sdAb or functional variant thereof is a codon-optimized polynucleotide. In some embodiments, the sdAb polynucleotide or sdAb codon-optimized polynucleotide comprises recombinant, engineered, or isolated forms of naturally occurring nucleic acids isolated from an organism, e.g., dromedaries, camels, llamas, alpacas or sharks.

Exemplary polynucleotides that encode a fusion protein of the disclosure include those set forth in SEQ ID NOs: 8, 10, 12, 14, 16, 18, 20, and 195-198.

The term “host cell” as used herein, includes any cell that is susceptible to transformation with a nucleic acid construct. The term “transformation” means the introduction of a foreign (i.e., extrinsic or extracellular) gene, DNA, or RNA sequence to a host cell, so that the host cell will express the introduced gene or sequence to produce a desired substance, typically a protein or enzyme coded by the introduced gene or sequence. The introduced gene or sequence may include regulatory or control sequences, such as start, stop, promoter, signal, secretion, or other sequences used by the genetic machinery of the cell. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species, or by gene synthesis.

The term “codon-optimized sequences” generally refers to nucleotide sequences that have been optimized for a particular host species by replacing any codons having a usage frequency of less than about 20%. Nucleotide sequences that have been optimized for expression in a given host species by, for example, elimination of spurious polyadenylation sequences, elimination of exon/intron splicing signals, elimination of transposon-like repeats, and/or optimization of GC content in addition to codon optimization are referred to herein as “expression enhanced sequences.”

The disclosure further provides a vector comprising one or more nucleic acid sequences encoding the disclosed CD proteins or functional variants thereof, sdAbs or functional variants thereof, linkers, and/or fusion proteins. The vector can be, for example, a plasmid, episome, cosmid, viral vector (e.g., retroviral or adenoviral), or phage. Suitable vectors and methods of vector preparation are well known in the art (see, e.g., Sambrook et al., Molecular Cloning, a Laboratory Manual, 4th edition, Cold Spring Harbor Press, Cold Spring Harbor, N.Y. (2012), and Ausubel et al., Current Protocols in Molecular Biology, Greene Publishing Associates and John Wiley & Sons, New York, N.Y. (1994, and updated chapters available online)).

In some embodiments, a vector comprising one or more nucleic acids encoding one or more amino acid sequences disclosed herein can be introduced into a host cell that is capable of expressing the polypeptides/proteins encoded thereby, including any suitable prokaryotic or eukaryotic cell. As such, the disclosure provides a cell (including an isolated cell) comprising a disclosed vector. Preferred host cells are those that can be easily and reliably grown, have reasonably fast growth rates, have well characterized expression systems, and can be transformed or transfected easily and efficiently.

Examples of suitable prokaryotic host cells include, but are not limited to, cells from the genera Bacillus (such as Bacillus subtilis and Bacillus brevis), Escherichia (such as E. coli), Pseudomonas, Streptomyces, Salmonella, and Erwinia. Additional suitable prokaryotic host cells include the various strains of Escherichia coli (e.g., K12, HB101 (ATCC No. 33694), DHS, DH10, MC1061 (ATCC No. 53338), and CC102). In some embodiments, the host cell is Tuner™ (Novagen), AD494 (Novagen), HMS174 (Novagen), NovaBlue (Novagen), BLR (Novagen), C41 (Lucigen), C43 (Lucigen), Lemo21 (NEB), NiCo21 (NEB), BL21, BL21(DE3), or T7 Express (NEB).

In some embodiments, the host cell is an E. coli strain that provides a cytoplasmic environment for disulfide bond formation. For example, the cytoplasmic environment is achieved by optimizing the thioredoxin and/or glutathione pathway, and/or by expressing cytosolic disulfide bond isomerase. In some embodiments, the host cell is an E. coli strain that constitutively expresses a chromosomal copy of a cytosolic disulfide bond isomerase (e.g., DsbC). In some embodiments, the prokaryotic host cells are SHuffle® Express (NEB#C3028) cells (New England Biolabs). In some embodiments, the prokaryotic host cells are SHuffle® T7 (NEB #C3026) or SHuffle® Express T7 LysY (NEB#C3030) cells. SHuffle® has deletions of the genes for glutaredoxin reductase and thioredoxin reductase (Agor AtrxB), which allow disulfide bonds to form in the cytoplasm. This combination of mutations is normally lethal, but the lethality is suppressed by a mutation in the gene encoding peroxiredoxin enzyme (ahpC*). In addition, SHuffle® expresses a version of the periplasmic disulfide bond isomerase DsbC that lacks its signal sequence, retaining DsbC in the cytoplasm. This enzyme has been shown to act on proteins with multiple disulfide bonds to correct mis-oxidized bonds and promote proper folding. Any other cell line with these properties (e.g., providing a cytoplasmic environment for disulfide bond formation) can be used to prepare the compounds of the disclosure. In some embodiments, the prokaryotic host cells are Origami™ or Rosetta-gami™.

Examples of yeast eukaryotic expression system include, but are not limited to, genera Saccharomyces, Pichia, Kluyveromyces, Hansenula, and Yarrowia.

Suitable insect host cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Exemplary insect host cells include Sf-9 and HIS (Invitrogen, Carlsbad, Calif.).

Examples of in vitro protein expression include, but are not limited to, E. coli lysates, rabbit reticulocyte lysates (RRL), wheat germ extracts, and insect cell lysates (such as SF9 or SF21 lysates). Cell-free expression system is the production of recombinant proteins in which the protein synthesis occurs in cell lysates rather than within cultured cells. A cell-free expression system can provide several advantages and features that complement traditional in vivo methods, such as faster production speed, because it does not require gene transfection, cell culture, or extensive protein purification.

Combination Treatments with Other Drugs

The fusion proteins of the disclosure can be administered alone or in combination with other drugs (e.g., as an adjuvant). For example, numerous chemotherapeutics, especially antineoplastic drugs, are available for combination with the fusion proteins of the disclosure. Most chemotherapeutic drugs can be divided into alkylating agents, antimetabolites, anthracyclines, plant alkaloids, topoisomerase inhibitors, antibodies, and other anti-tumor agents.

As used herein, adjunctive or combined administration (co-administration) includes simultaneous administration of a fusion protein and another drug in the same or different dosage form, or separate administration of fusion protein and another drug (e.g., sequential administration).

In certain embodiments, the fusion proteins of the disclosure are co-administered with an antineoplastic drug that damages DNA or interferes with DNA repair. In some embodiments, the fusion proteins of the disclosure and an antineoplastic drug act synergistically. In some embodiments, the fusion proteins of the disclosure increase a cell's sensitivity to the antineoplastic drug, for example, by at least 10%, 15%, 20%, 25%, 30%, 35%, 40%, 45%, or 50%. Non-limiting examples of antineoplastic drugs that damage DNA or inhibit DNA repair include carboplatin, carmustine, chlorambucil, cisplatin, cyclophosphamide, dacarbazine, daunorubicin, doxorubicin, epirubicin, idarubicin, ifosfamide, lomustine, mechlorethamine, mitoxantrone, oxaliplatin, procarbazine, temozolomide, and valrubicin. In some embodiments, the antineoplastic drug is temozolomide, which is a DNA damaging alkylating agent commonly used against glioblastomas. In some embodiments, the antineoplastic drug is a PARP inhibitor (e.g., KU0058948, ABT-888 (veliparib), olaparib, KU-59436, AZD-2281, AG-014699, BSI-201, BGP-15, INO-1001, ONO-2231), which inhibits a step in base excision repair of DNA damage. In some embodiments, the antineoplastic drug is a histone deacetylase inhibitor (e.g., Vorinostat; Romidepsin; Chidamide; Panobinostat; Valproic acid; Belinostat; Mocetinostat; Abexinostat; Entinostat; SB939 (pracinostat); Resminostat; Givinostat; Quisinostat; thioureidobutyronitrile (Kevetrin™); CUDC-10; CHR-2845 (tefinostat); CHR-3996; 4SC-202; CG200745; ACY-1215 (rocilinostat); ME-344; sulforaphane), which suppresses DNA repair at the transcriptional level and disrupts chromatin structure. In some embodiments, the antineoplastic drug is a proteasome inhibitor (e.g., Bortezomib; Carfilzomib; Epoxomicin; Ixazomib; Salinosporamide A), which suppresses DNA repair by disrupting ubiquitin metabolism in the cell. Ubiquitin is a signaling molecule that regulates DNA repair. In some embodiments, the antineoplastic drug is a kinase inhibitor (e.g., an ATM inhibitor (CP466722 or KU-55933); a CHK 1 inhibitor (XL-844, UCN-01, AZD7762 or PF00477736), or a CHK 2 inhibitor (XL-844, AZD7762, or PF00477736)), which suppresses DNA repair by altering DNA damage response signaling pathways.

Additional examples of antineoplastic drugs that can be combined with the fusion proteins of the disclosure include, but are not limited to, alkylating agents (such as temozolomide, cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as gemcitabine, methotrexate, cytosine arabinoside, fludarabine, and floxuridine), antimitotics, vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (including camptothecins such as topotecan and derivatives of epipodophyllotoxins such as amsacrine, etoposide, etoposide phosphate, and teniposide).

Examples of other chemotherapeutic agents that may be administered in combination with fusion proteins of the disclosure include alkylating agents such as thiotepa and cyclophosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (e.g., bullatacin and bullatacinone); delta-9-tetrahydrocannabinol (dronabinol); beta-lapachone; lapachol; colchicines; betulinic acid; a camptothecin (including the synthetic analogue topotecan (CPT-11 (irinotecan), acetylcamptothecin, scopolectin, and 9-aminocamptothecin); bryostatin; pemetrexed; callystatin; CC-1065 (including its adozelesin, carzelesin, and bizelesin synthetic analogues); podophyllotoxin; podophyllinic acid; teniposide; cryptophycins (e.g., cryptophyan 1 and cryptophycin 8); dolastatin; duocarmycin (including the synthetic analogues, KW-2189 and CB1-TM1), eleutherobin; pancratistatin; TLK-286; CDP323, an oral alpha-4 integrin inhibitor; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall, and calicheamicin omegall (see, e.g., Nicolaou et ah, Angew. Chem Intl. Ed. Engl., 33: 183-186 (1994)); dynemicin, including dynamics A; an esperamicin; neocarzinostatin chromophore and related chromoprotein enediyne antibiotic chromophores; aclacinomysins; actinomycin; authramycin; azaserine; bleomycins; cactinomycin; carabicin; carminomycin; carzinophilin; chromomycinis; dactinomycin; daunorubicin; detorubicin; 6-diazo-5-oxo-L-norleucine; doxorubicin (including morpholino-doxorubicin, cyanomorpholino-doxorubicin, 2-pyrrolino-doxorubicin, doxorubicin HCl liposome injection and deoxydoxorubicin); epirubicin; esorubicin; idarubicin; marcellomycin; mitomycins such as mitomycin C, mycophenolic acid, nogalamycin, olivomycins, peplomycin, potfiromycin, puromycin, guelamycin, rodorubicin, streptonigrin, streptozocin, tubercidin, ubenimex, zinostatin, and zorubicin; anti-metabolites such as methotrexate, gemcitabine, tegafur, capecitabine, an epothilone, and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid, triaziquone; 2,2,2-trichlorothethylamine; trichothecenes (e.g., T-2 toxin, verracurin A, roridin A, and anguidine); urethan; vindesine; dacarbazine; mannomustine; mitobronitol; mitolactol; pipobroman; gacytosine; arabinoside (“Ara-C”); thiotepa; taxoids, e.g., paclitaxel, albumin-engineered nanoparticle formulation of paclitaxel, and docetaxel; chloranbucil; 6-thioguanine; mercaptopurine; methotrexate; platinum analogs such as cisplatin and carboplatin; vinblastine; platinum; etoposide (VP-16); ifosfamide; mitoxantrone; vincristine; oxaliplatin; leucovovin; vinorelbine; novantrone; edatrexate; daunomycin; aminopterin; ibandronate; topoisomerase inhibitor RFS 2000; difluoromethylomithine (DMFO); retinoids such as retinoic acid; pharmaceutical acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin combined with 5-FU and leucovovin. Other therapeutic agents that may be used in combination with the fusion proteins of the disclosure include bisphosphonates such as clodronate, NE-58095, zoledronio acid/zoledronate, alendronate, pamidronate, tiludronate, or risedronate; as well as troxacitabine (a 1,3-dioxolane nucleoside cytosine analog), anti-sense oligonucleotides, particularly those that inhibit expression or genes in signaling pathways implicated in aberrant cell proliferation, such as, for example, PKC-alpha, Raf, H-Ras, and epidermal growth factor receptor (EGFR); vaccines such as Stimuvax vaccine, Theratope vaccine and gene therapy vaccines, for example, Allovectin vaccine, Leavectin vaccine, and Vaxid vaccine; topoisomerase 1 inhibitor; an anti-estrogen such as fulvestrant; a Kit inhibitor such as imatinib or EXEL-0862 (a tyrosine kinase inhibitor); EGFR inhibitor such as erlotinib or cetuximab; an anti-VEGF inhibitor such as bevacizumab; arinotecan; rmRH; lapatinib and lapatinib ditosylate (an ErbB-2 and EGFR dual tyrosine kinase small-molecule inhibitor also known as GW572016); 17AAG (geldanamycin derivative that is a heat shock protein (Hsp) 90 poison), and pharmaceutical acceptable salts, acids or derivatives of any of the above.

In some embodiments, the fusion proteins or pharmaceutical compositions disclosed herein are co-administrated with 5-fluorouracil (5-FU). In some embodiments, the fusion proteins or pharmaceutical compositions thereof are co-administered with a 5-FU containing regimen for example: FOLFUHD (5-FU and leucovorin, sLV5FU2 (5-FU and leucovorin), IFL (irinotecan, leucovorin, and 5-FU), FLOX (5-FU, leucovorin, and oxaliplatin), mFOLFOX6 (oxaliplatin, leucovorin, and 5-FU), FOLFOX4 (oxaliplatin, leucovorin, and 5-FU), FOLFOX7 (oxaliplatin, leucovorin and 5-FU), FOLFIRI (irinotecan, leucovorin, and 5-FU), FOLFOXIRI (irinotecan, oxaliplatin, leucovorin, and 5-FU), FOLFIRINOX (Leucovorin Calcium, Fluorouracil, Irinotecan Hydrochloride, and Oxaliplatin), or CMF (cyclophosphamide, methotrexate, and 5-FU). In some embodiments, the fusion proteins or pharmaceutical compositions thereof are co-administered with a 5-FC containing regimen. For example, in some embodiments, the 5-FU component of any of the aforementioned 5-FU containing regimens is replaced with 5-FC (e.g., 5-FC and leucovorin; 5-FC, leucovorin, and irinotecan; 5-FC, leucovorin, and oxaliplatin; 5-FU, leucovorin, irinotecan, and oxaliplatin; 5-FU, leucovorin calcium, irinotecan hydrochloride, and oxaliplatin; 5-FC, cyclophosphamide, and methotrexate).

In some embodiments, fusion proteins or pharmaceutical compositions of the disclosure can be combined with or co-administered with one or more substances that potentiate the cytotoxic effect of the 5-FU. Substances that potentiate the cytotoxic effect of 5-FU include, but are not limited to, drugs that inhibit enzymes of the de novo biosynthesis of the pyrimidines; and drugs, such as Leucovorin (Waxman et al., 1982, Eur. J. Cancer Clin. Oncol. 18, 685-692), which, in the presence of the product of the metabolism of 5-FU (5-FdUMP), increases the inhibition of thymidylate synthase, resulting in a decrease in the pool of dTMP, which is required for replication; and drugs such as methotrexate (Cadman et al., 1979, Science 250, 1135-1137) which, by inhibiting dihydrofolate reductase and increasing the pool of PRPP (phosphoribosylpyrophosphate), bring about an increase in the incorporation of 5-FU into cellular RNA. In some embodiments, the substances that potentiate the cytotoxic effect of 5-FU inhibit the degradation of 5-FU. For example, in some embodiments, the substance is Gimeracil. In some embodiments, the substances that potentiate the cytotoxic effect of 5-FU decrease the side effect(s) of 5-FU. For example, in some embodiments, the substance is Oteracil potassium. In some embodiments, the substances that potentiate the cytotoxic effect of 5-FU inhibit the metabolism of 5-FU by inhibiting dihydropyrimidine dehydrogenase. For example, in some embodiments, the substance is uracil.

### Methods of Administration

Delivery of the fusion proteins or pharmaceutical compositions of the disclosure can be conducted in different ways, including oral, subcutaneous, intravenous, intraperitoneal, or intratumoral administration. Other administration and delivery routes include intra-articular, intra-arterial, intramuscular, parenteral, subcutaneous, intra-pleural, topical, dermal, intradermal, transdermal, parenteral, e.g. transmucosal, intra-cranial, intra-spinal, mucosal, respiratory, intranasal, via intubation, intrapulmonary, intrapulmonary instillation, buccal, sublingual, intravascular, intrathecal, intracavity, iontophoretic, intraocular, ophthalmic, intraglandular, intraorgan, and intralymphatic.

Each delivery/administration pathway has different demands for a fusion protein formulation according to the disclosure, and the formulations can be prepared routinely by one of ordinary skill in the art. For instance, with the oral application or the intraperitoneal injection, the sdAb-CD fusion protein requires resistance to extreme conditions (i.e., proteases and/or acidic pH). If needed, the fusion proteins can be made resistant to proteases by adaptation of the sequence or by the introduction of an additional disulfide bond in order to improve resistance to pepsin and chymotrypsin, as is well known in the art. For intravenous injection, stability in serum can be important. Most sdAbs combined with effector domains or nanoparticles have been described as very stable in serum.

According to some embodiments, the therapeutic use or the treatment method comprises an additional step in which pharmaceutically acceptable quantities of a prodrug, such as an analog of cytosine, in particular 5-FC, are administered to the subject or cell. By way of non-limiting illustration, it is possible to use a dose of from 50 to 1000 mg/kg/day, or a dose of 500 mg/kg/day or a dose of 200 mg/kg/day, one time a day or more than one time a day. In some embodiments, the method includes at least a first loading dose of 5-FC sufficient to obtain a serum concentration of about 1-200 (e.g., 10-100) μg/ml within 1-2 days of administration. In some embodiments, the prodrug is administered in accordance with standard practice (e.g., orally, systematically), with the administration taking place subsequent to the administration of a fusion protein disclosed herein. In some embodiments, the prodrug is administered orally. In some embodiments, the prodrug is administered in a single dose. In some embodiments, the prodrug is administered in doses that are repeated for a time sufficient to enable the toxic metabolite to be produced within the host organism or cell.

In some embodiments, the prodrug is a compound that can be converted in vivo to provide a biologically, pharmaceutically, or therapeutically active form of 5-FC. Such photoactivatable compounds can comprise a photosensitive linker that is cleavable upon irradiation with, for example, a UV light (including lights implanted within a tumor site that can be remotely or temporally activated).

In some embodiments, a cytosine analog is administered instead of 5-FC. Cytosine analogs that can be substrates for cytosine deaminase include halogenated cytosines and the prodrug 5-fluorocytosine (5-FC) (which is activated by CD to 5-fluorouracil (5-FU)). In addition, extended release formulations of 5-FC can be used (e.g., Toca FC).

### Pharmaceutical Compositions

The disclosure provides pharmaceutical compositions comprising one or more of the fusion proteins disclosed herein. In some embodiments, the pharmaceutical composition comprises one or more of the fusion proteins disclosed herein and one or more pharmaceutically acceptable excipients. Pharmaceutical compositions comprising a fusion protein of the disclosure and any post-translational modifications thereof and a pharmaceutically acceptable excipient are also within the scope of this disclosure and may be prepared using methods known in the art. Suitable excipients are well known in the art. The choice of excipient will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 22nd Edition, Lippincott Williams & Wilkins, Philadelphia, Pa. (2013) and any other editions.

The term “excipient” broadly refers to any component other than the active therapeutic ingredient(s). The excipient may be an inert substance, an inactive substance, and/or a not medicinally active substance. The excipient may serve various purposes, e.g. as a carrier, vehicle, diluent, tablet aid, and/or to improve administration, and/or absorption of the active substance.

In some embodiments, the pharmaceutical compositions of the disclosure are prepared to have certain stabilities (physical and/or chemical stability). The term “physical stability” refers to the tendency of a polypeptide or protein to form biologically inactive and/or insoluble aggregates as a result of exposure to thermo-mechanical stress, and/or interaction with destabilizing interfaces and surfaces (such as hydrophobic surfaces). The physical stability of an aqueous protein formulation may be evaluated by means of visual inspection, and/or by turbidity measurements after exposure to mechanical/physical stress (e.g. agitation) at different temperatures for various time periods. Alternatively, the physical stability may be evaluated using a spectroscopic agent or probe of the conformational status of the protein such as e.g., Thioflavin T or “hydrophobic patch” probes.

The term “chemical stability” refers to chemical (in particular covalent) changes in the polypeptide or protein structure leading to formation of chemical degradation products potentially having a reduced biological potency, and/or increased immunogenic effect as compared to the intact protein. The chemical stability can be evaluated by measuring the amount of chemical degradation products at various time-points after exposure to different environmental conditions, e.g., by SEC-HPLC and/or RP-HPLC.

The fusion proteins of the disclosure can be used therapeutically as pharmaceutical formulations. The term “pharmaceutical formulation” refers to a preparation that is in such form as to permit the biological activity of the active ingredient to be effective, wherein the formulation does not comprise additional components that are unacceptably toxic to a subject to whom the formulation would be administered. In some embodiments, the pharmaceutical formulations are sterile.

In some embodiments, a pharmaceutical composition or pharmaceutical formulation may be a solution, emulsion, or suspension (for example, incorporated into microparticles, liposomes, or cells). Typically, an appropriate amount of a pharmaceutically acceptable salt is used in the composition or formulation to render it isotonic. Examples of pharmaceutically acceptable carriers include, but are not limited to, saline, Ringer's solution, and dextrose solution. The pH of the solution is preferably from about 5 to about 8, and more preferably from about 7 to about 7.5. Pharmaceutical compositions or formulations may include carriers, thickeners, diluents, buffers, preservatives, and/or surface active agents. Suitable carriers include sustained release preparations, such as semi-permeable matrices of solid hydrophobic polymers containing the fusion protein of the disclosure, which matrices are in the form of shaped particles, e.g., films, liposomes, or microparticles. It will be apparent to those persons skilled in the art that certain carriers may be more preferable depending upon, for instance, the route of administration and concentration of the composition being administered. Pharmaceutical compositions or formulations may also include one or more additional active ingredients such as cytotoxic agents, cytostatic agents, chemotherapeutic agents, antimicrobial agents, anti-inflammatory agents, and anesthetics.

To aid dissolution of the fusion proteins of the disclosure into the aqueous environment a surfactant might be added as a wetting agent. Surfactants may include anionic detergents such as sodium lauryl sulfate, dioctyl sodium sulfosuccinate, and dioctyl sodium sulfonate. Cationic detergents might be used and could include benzalkonium chloride or benzethonium chloride. Nonionic detergents that could be used in the formulation as surfactants include, but are not limited to, lauromacrogol 400; polyoxyl 40 stearate; polyoxyethylene hydrogenated castor oil 10, 50, or 60; glycerol monostearate; polysorbate 20, 40, 60, 65, or 80; sucrose fatty acid ester; methyl cellulose; and carboxymethyl cellulose. These surfactants could be present in the pharmaceutical composition or formulation of the fusion protein either alone or as a mixture in different ratios. Additives that may enhance uptake of peptides may be included in a pharmaceutical composition or formulation of the disclosure. For instance, in some embodiments, the composition or formulation includes one or more of the fatty acids oleic acid, linoleic acid, and linolenic acid.

### Methods of Using

The fusion proteins of the disclosure can be used in, for example, the treatment of proliferative diseases (cancers/tumors, restenosis, glaucoma, scarring). In some aspects, the disclosure pertains to a method comprising administering a fusion protein or pharmaceutical composition or formulation thereof to a subject having a cancer or any disease disclosed herein, whereupon the disease is treated in the subject. In addition to therapeutic uses, the fusion proteins, pharmaceutical compositions, and/or pharmaceutical formulations described herein can be used in diagnostic or research applications.

In some embodiments, the fusion proteins of the disclosure, or pharmaceutical composition or formulations thereof, can be used to treat any cancer known in the art such as colon cancer, esophagus cancer, stomach cancer, pancreatic cancer, breast cancer, basal cell carcinoma, Bowen's disease, and cervical cancer. In some embodiments, the compounds of the disclosure can be used to treat ocular surface squamous neoplasia. In some embodiments, the disclosed fusion proteins, pharmaceutical compositions, and/or pharmaceutical formulations can be used to treat melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, cholangiocarcinoma, esophagus cancer, bone cancer, endometrial cancer, ovarian cancer, soft tissue sarcoma, or Merkel cell carcinoma. In some embodiments, the disclosed fusion proteins, pharmaceutical compositions, and/or pharmaceutical formulations can be used to treat a solid tumor. In some embodiments, the solid tumor is colon cancer, colorectal cancer, pancreatic cancer, or head and neck cancer.

In some embodiments, the disclosed fusion proteins, pharmaceutical compositions, and/or pharmaceutical formulations can be used to treat actinic keratosis. In some embodiments, the disclosed fusion proteins, pharmaceutical compositions, and/or pharmaceutical formulations can be used as an adjunctive therapy in ocular and/or periorbital surgeries. In some embodiments, the disclosed fusion proteins, pharmaceutical compositions, and/or pharmaceutical formulations can be used in the treatment of hypertrophic (HTSs) and/or keloid scars.

The term “treatment” (or “treat”) refers to the medical management of a patient with the intent to cure, ameliorate, or stabilize a disease. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and/or supportive treatment, that is, treatment employed to supplement another specific therapy directed toward the improvement of the associated disease, pathological condition, or disorder.

The term “therapeutically effective” means that the amount of the protein, composition, or formulation used is of sufficient quantity to ameliorate one or more causes or symptoms of a disease or disorder. Such amelioration only requires a reduction or alteration, not necessarily elimination. A therapeutically effective amount of a protein, composition, or formulation for treating cancer is preferably an amount sufficient to cause tumor regression or to sensitize a tumor to radiation or chemotherapy. The amount of the disclosed fusion proteins, pharmaceutical compositions, and/or pharmaceutical formulations that is required for use in treatment will vary not only with the particular sdAb (or functional variant thereof) and fusion protein selected but also with the route of administration, the nature of the condition being treated, and the age and condition of the patient, among other factors, and will be ultimately at the discretion of the attendant physician or clinician. Also, the dosage of the disclosed fusion proteins, pharmaceutical compositions, and/or pharmaceutical formulations may vary depending on the target cell, tumor, tissue, graft, or organ.

Clinicians will generally be able to determine a suitable dose, depending on the factors mentioned herein. It will also be clear that in specific cases, the clinician may choose to deviate from these amounts, for example, on the basis of the factors cited above and his or her expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life, and similar factors well known to the skilled person. For example, the fusion proteins of the disclosure may generally be administered in an amount between 1 gram and 0.01 microgram per kg body weight per day, preferably between 0.1 gram and 0.1 microgram per kg body weight per day, such as about 1, 10, 100, or 1000 micrograms per kg body weight per day, either continuously (e.g., by infusion), as a single daily dose or as multiple divided doses during the day. In some embodiments, the fusion proteins of the disclosure are administered in an amount from about 10 mg/kg to about 60 mg/kg. In some embodiments, the fusion proteins of the disclosure are administered in an amount from about 10 mg/kg/day to about 60 mg/kg/day.

Other suitable doses for the fusion proteins of the disclosure can be, for example, in the range of 1 pg/kg to 60 mg/kg of animal or human body weight; however, doses below or above this exemplary range are within the scope of the invention. The daily parenteral dose can be about 0.00001 μg/kg to about 20 or about 40 mg/kg of total body weight (e.g., about 0.001 μg/kg, about 0.1 μg/kg, about 1 μg/kg, about 5 μg/kg, about 10 μg/kg, about 100 μg/kg, about 500 μg/kg, about 1 mg/kg, about 5 mg/kg, about 10 mg/kg, or a range defined by any two of the foregoing values), preferably from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), and even more preferably from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values). Therapeutic or prophylactic efficacy can be monitored by periodic assessment of treated patients. For repeated administrations over several days or longer, depending on the condition, the treatment can be repeated until a desired suppression of disease symptoms occurs. However, other dosage regimens may be useful and are within the scope of the disclosure. For example, the desired dosage can be delivered by a single bolus administration of the composition, by multiple bolus administrations of the composition, or by continuous infusion administration of the compositions of the disclosure. Other methods of administration that can be used with the fusion proteins disclosed herein are exemplified elsewhere in this document.

### EXAMPLES

### Example 1. Construction of Mammalian Expression Plasmids

The expression plasmids of CD-fusion proteins were constructed by recombinant DNA techniques routine in the art. The construction methods/design of some represented expression plasmid are described herein.

(1) Rituxan-CD-CD Expression Plasmid

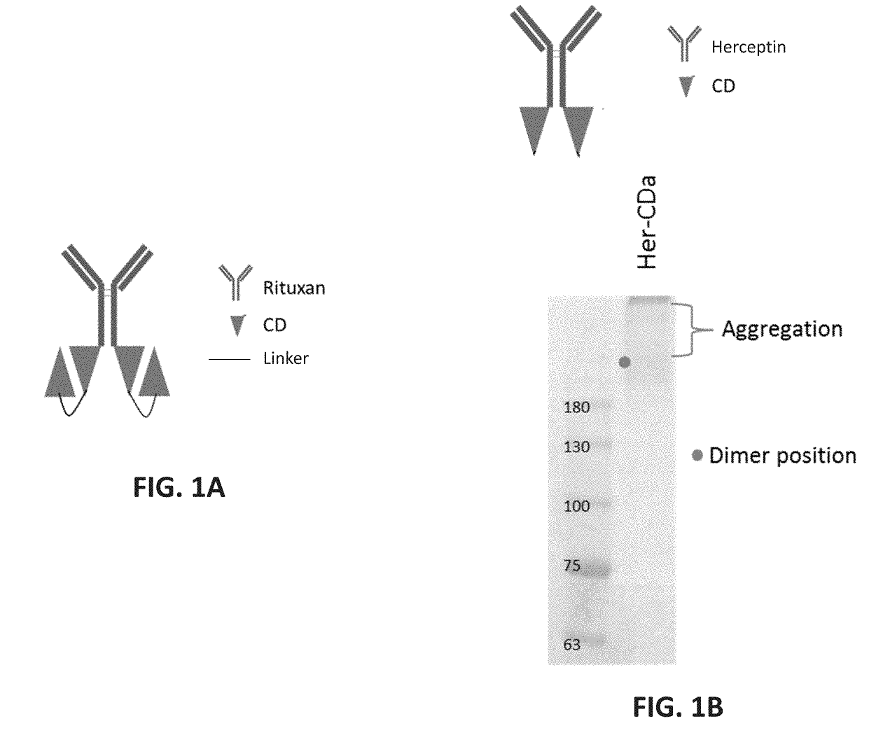

The design of a Rituxan-CD-CD fusion protein is shown in FIG. 1A. The nucleic acids sequence of Rituxan heavy chain, Rituxan light chain and yeast cytosine deaminase can be synthesized by gene synthesis. The coding fragment containing SP-RituxanHC-CD-linker-CD (SEQ ID NO: 89) and SP-RituxanLC (SEQ ID NO: 90) were generated by overlapping PCR. The fragment SP-RituxanHC-CD-linker-CD and SP-RituxanLC were than cloned into pCHO1.0 vector via AvrII/BstZ171 site and EcoRV/PacI site respectively. (*SP: signal peptide)

(2) Herceptin-CD Expression Plasmid

The design of a Herceptin-CD fusion protein is shown in FIG. 1B. The nucleic acids sequence of Herceptin variable region and yeast cytosine deaminase can be synthesized by gene synthesis. The coding fragment containing SP-HerceptinHC-CD (SEQ ID NO: 91) and SP-HerceptinLC (SEQ ID NO: 92) were generated by overlapping PCR. The fragment SP-HerceptinHC-CD and SP-HerceptinLC were than cloned into pCHO1.0 vector via AvrII/BstZ171 site and EcoRV/PacI site respectively.

### Example 2. Production of Rituxan-CD-CD and Herceptin-CD in Mammalian Cells

For production of mammalian expression protein, Rituxan-CD-CD and Herceptin-CD were transiently expressed by CHO-S™ cells (Thermo) using FreeStyle Max′ reagent according to the transfection protocol of FreeStyle Max™. Supernatants were harvested at 72 hours after transfection. The supernatants were then: (1) quantified by ELISA to determine protein titer, (2) purified by Protein A and expression pattern checked by non-reducing PAGE, and (3) concentrated for CD activity analysis.

The expression titers of Rituxan-CD-CD and Herceptin CD were 0.002 μg/mL and 0.5 μg/mL, respectively. Both fusion proteins had CD activity. Rituxan-CD-CD was almost un-detectable by PAGE even after Protein A purification (data not shown). It was observed that Herceptin-CD was aggregated as multimer when analyzed by non-reducing PAGE (FIG. 1B).

### Example 3. Construction of E. coli Expression Plasmids

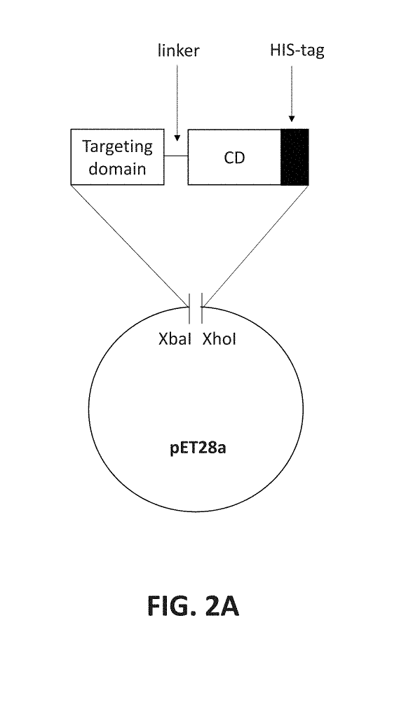

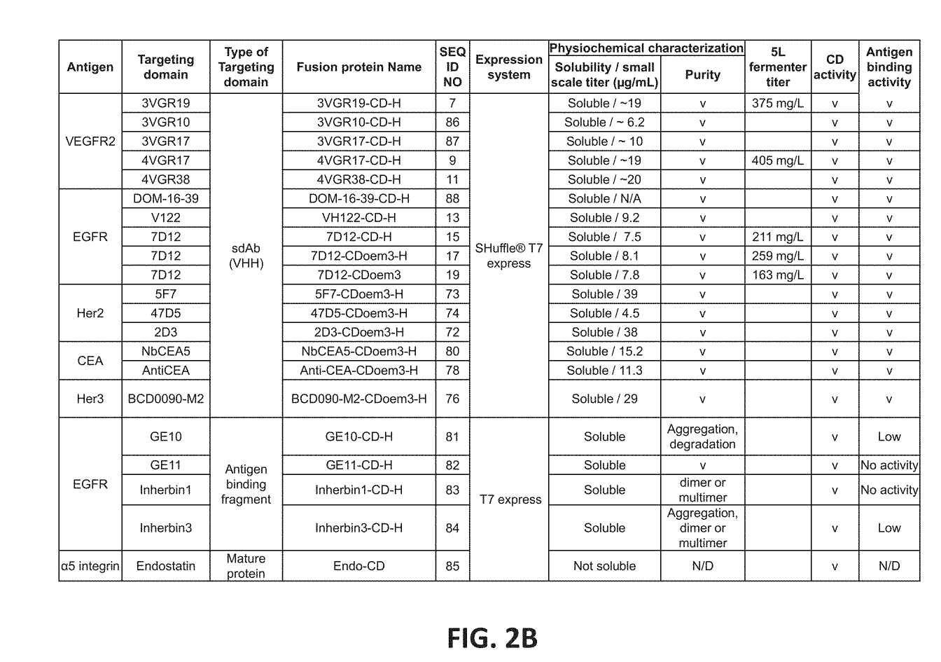

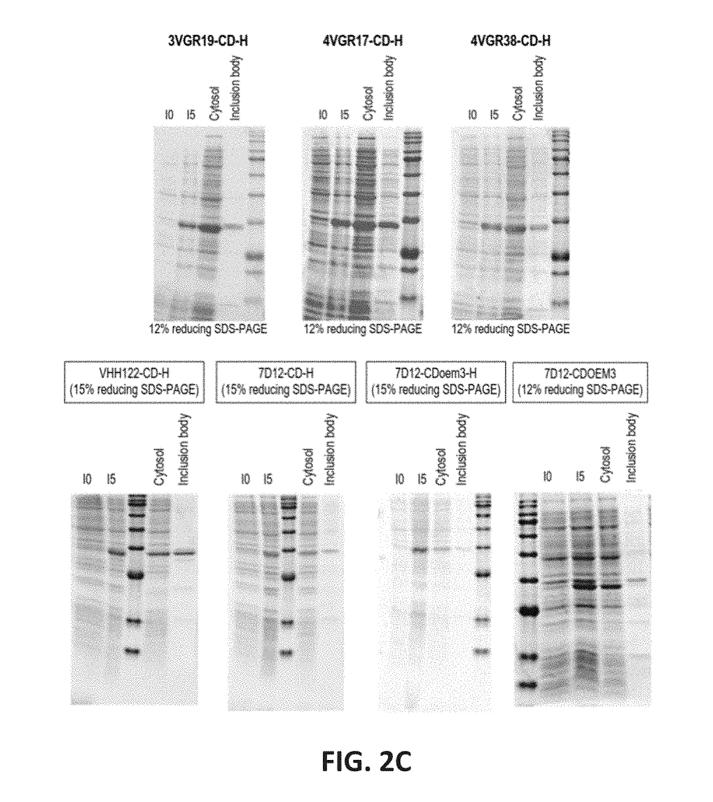

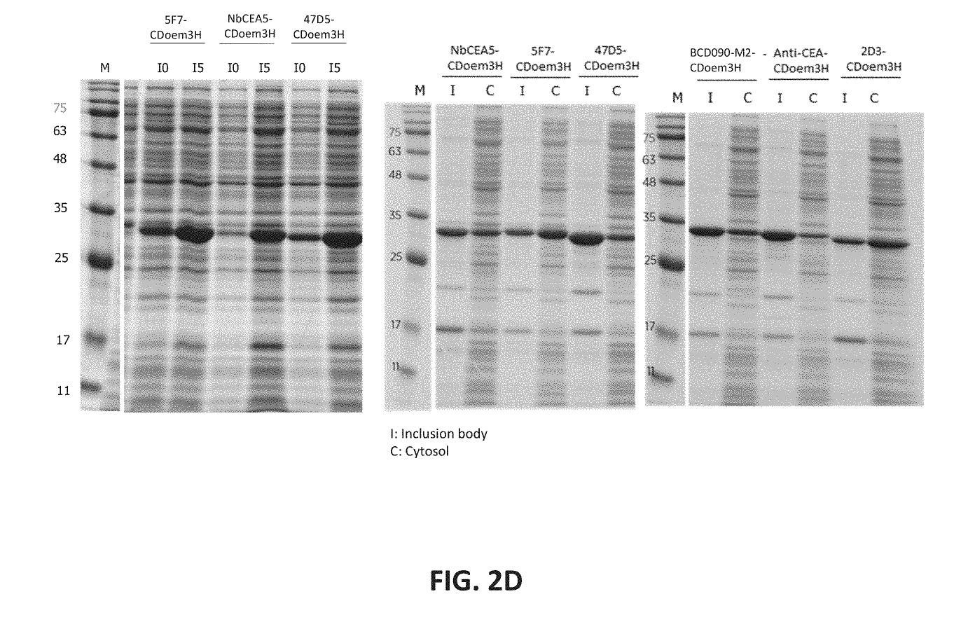

The coding sequence for each fusion protein comprises two main components, one encoding the antigen-recognition fragment (targeting domain), such as sdAb, antigen binding fragment, or endostatin; and one encoding the yeast cytosine deaminase fragment. A coding sequence encoding a linker peptide sequence connected the main components, such that, when expressed, the linker does not interfere with the function of either the targeting domain protein component or the CD component. The expression plasmid is simplified in the schematic shown in FIG. 2A.

(1) Single-Domain Antibody-CD Expression Plasmid

The nucleic acid sequences of sdAb and yeast cytosine deaminase can be synthesized by gene synthesis. The coding fragment of the sdAb-CD fusion proteins was obtained by overlapping PCR using specific primers. The linker peptide sequence between the sdAb and CD was (GGGGS)3 (SEQ ID NO: 188). The fragments were cloned into a pET28a vector (Novagen) via XbaI and XhoI sites (FIG. 2A). Fusion protein variants with CD mutation(s) or VHH mutation(s) were generated by overlapping PCR.

(2) Antigen-Binding Fragment-CD Expression Plasmid

The nucleic acid sequences of yeast cytosine deaminase were synthesized by gene synthesis. The coding fragment of antigen-binding fragment-CD (SEQ ID NOs: 81-84) was obtained by extension PCR using specific primers. The linker peptide sequence between the antigen-binding fragment and CD was (GGGGS)3 (SEQ ID NO: 188). The fragments were cloned into pET28a vector (Novagen) via XbaI and XhoI sites (FIG. 2A).

(3) Endostatin-CD Expression Plasmid

The nucleic acid sequences of endostatin and yeast cytosine deaminase were synthesized by gene synthesis. The coding fragment of endostatin-CD fusion proteins (SEQ ID NO: 85) was obtained by overlapping PCR using specific primers. The linker peptide sequence placed in between antigen binding fragment and CD was (GGGGS)3 (SEQ ID NO: 188). The fragments were than cloned into pET28a vector (Novagen) via XbaI and XhoI sites (FIG. 2A).

### Example 4. Production of CD Fusion Proteins in E. coli

For production, the expression plasmid was transformed into SHuffle® T7 express or T7 express competent E. coli cells (New England Biolabs) by a standard transformation procedure.

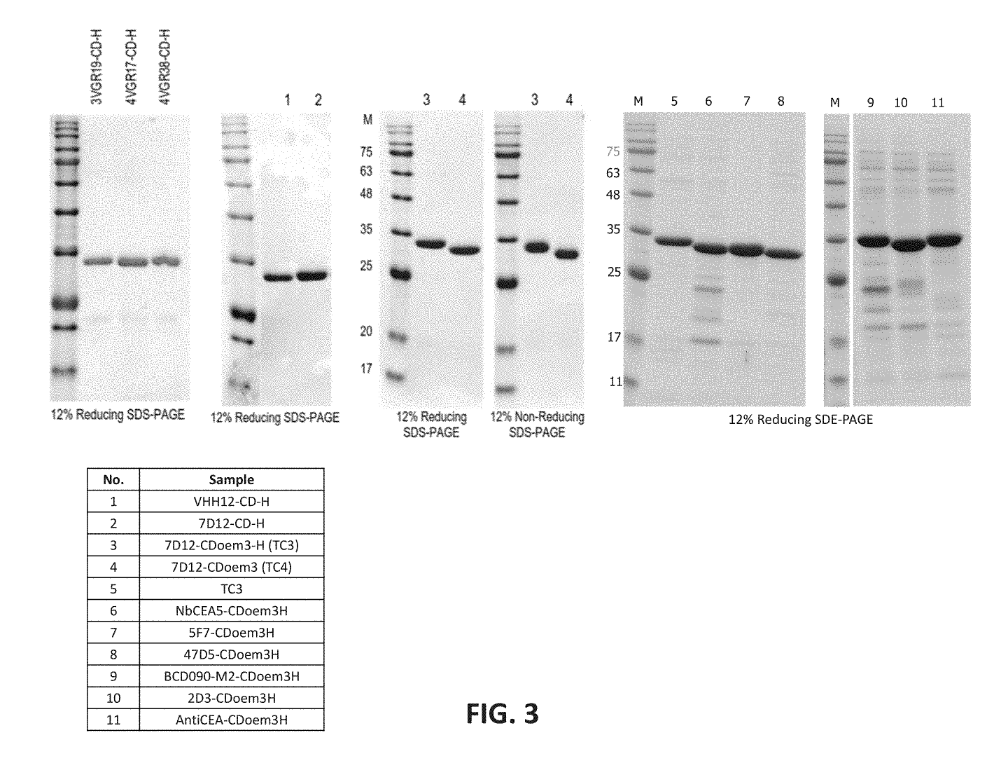

For pilot estimation of protein characteristics, the recombinant proteins were expressed and purified in about 300 mL production scale. For protein expression, the refreshed transformants was inoculated at 1:100 dilution in the selection medium at 30° C. (SHuffle® T7 Express cells) or 37° C. (T7 Express cells) until OD600 reached 0.4-0.8 (Jo). IPTG (Uni-region) at 0.4 mM was added to induce protein expression. For T7 Express cells, the induced culture was incubated at 37° C. for 5 hours. For SHuffle® T7 Express cells, after induction temperature for production was 25° C. or 30° C., the temperature for production was 25° The induction temperature was 30° C. Five hours post induction (I5), the culture was harvested and resuspended in PBS for cell lysis by sonication. The soluble and insoluble fractions were collected from cell lysate separately. The soluble fraction of the cell lysate was firstly incubated with Ni Sepharose beads (GE Healthcare) at room temperature for 1 hour. After incubation, Ni beads (GE) was washed with 20 mM, 40 mM and 80 mM imidazole gradient (W1, W2 and W3, respectively). Each fusion protein was eluted with buffer containing 150 mM and 250 mM imidazole (E1 and E2). The samples from each step were analyzed by SDS-PAGE to assess the expression and purification profile. The purified recombinant proteins were collected and exchanged to PBS with 5% glycerol for SDS-PAGE, SEC-HPLC, CD activity and antigen-binding activity analysis. The expression titer, physiochemical characteristics, CD activity and antigen-binding activity of all recombinant proteins were estimated and are summarized in FIG. 2B. The expression profiles are exemplified in FIG. 2C and FIG. 2D. The SDS-PAGE, SEC-HPLC, CD activity and antigen binding activity analysis of purified sdAb-CD are exemplified in FIGS. 3, 4A, 4B, 5A, 5B, 6A, and 6B.

The results show that the sdAb-CD fusion proteins are mostly expressed in soluble form and exhibit acceptable purity profile during SDS-PAGE and SEC-HPLC analysis. The titer of sdAb-CD fusion proteins during small scale production were about or exceed 10 μg/mL, the titer can be further increased to 160-400 mg/L in large scale production, and may be increased to >1 g/L after process optimization, which are suitable for industrial production. The antigen-binding activity and CD activity were maintained in all sdAb-CD fusion proteins.

In contrast, endostatin-CD could not be expressed in soluble form, thus is not suitable for industrial production. The antigen-binding fragment-CD fusion proteins, though with smaller molecular weight, seemed to be expressed in soluble form. Most of the antigen-binding fragment-CD fusion proteins were aggregated as dimer or multimer form, and the antigen-binding fragments lost their antigen-binding activity when fused with CD.

More than ten sdAb-CD fusion proteins and five targeting antigens were tested, and the data showed that sdAb is a suitable antigen-binding fragment for fusion with CD, with both sdAb and CD retaining their biological activity.

### Example 5. Stability Study of sdAb-CD Produced from Different E. coli Strain

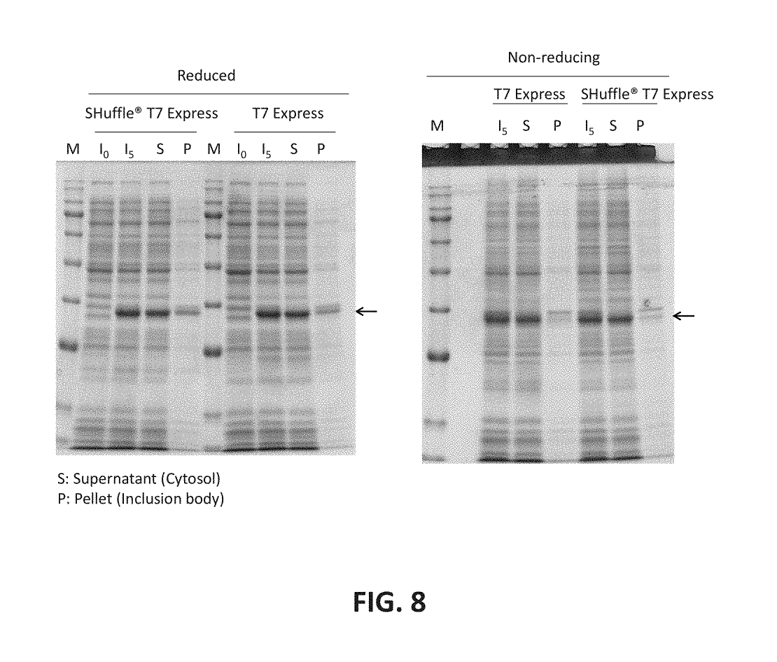

Expression plasmids of 3VGR19-CD-H were transformed into SHuffle® T7 Express and T7 Express Competent E. coli, respectively. Data suggested that the expression profile of 3VGR19-CD-H in SHuffle® T7 Express and T7 Express was similar (FIG. 8).

The 3VGR19-CD-H fusion proteins expressed from SHuffle® T7 Express and T7 Express Competent E. coli were purified and dialyzed into the following four buffers at 3 mg/mL: (1) PBS (2) 1% glycerol in PBS (3) 5% glycerol in PBS (4) 3% mannitol in PBS. The appearances of the purified protein in each buffer were observed after incubation at 37° C. for 3 hours, 37° C. for 8 hours and 4° C. for 1 week, and summarized in Table 1. The number of “+” indicates the turbidity level. “+/−” means that very minor aggregation was observed. The results showed that a large quantity of precipitations was observed in 3VGR19-CD-H expressed from T7 Express cells, but not in SHuffle® T7 Express cells.

TABLE 1
Stability of sdAb-CD produced from T7 Express
and SHuffle ® T7 Express

| Turbidity | T7 Express | | | | SHuffle ® T7 Express | | | |
|---|---|---|---|---|---|---|---|---|
| buffer | 1 | 2 | 3 | 4 | 1 | 2 | 3 | 4 |
| 37° C., | +++++ | +++++ | +++++ | +++++ | − | − | − | − |
| 3 hr | | | | | | | | |
| 37° C., | +++++ | +++++ | +++++ | +++++ | − | − | − | − |
| 8 hr | | | | | | | | |
| 4° C., | +++ | +++ | + | ++ | +/− | +/− | +/− | +/− |
| 1 week | | | | | | | | |

### Example 6. 5 L Fed-Batch Fermentation of sdAb-CD Fusion Proteins

To assess the production of sdAb-CD fusion proteins of the disclosure in bioreactor conditions, a 5 L fed-batch fermentation run was conducted. Frozen cells were inoculated into 2 mL selection medium at 30° C. for 4-6 hours, and then inoculated at 1:1000 dilution into the 200 mL selection medium as seed culture. The next day, seed culture was inoculated into the 5-L fermenter. The temperature was set at 30° C., pH at 7.2±0.1, DO at 25%, gasflow at 1-1.5 vvm. The feeding was started when glucose was below 0.3 g/L, and the feeding rate was adjusted to maintain the glucose at 0.5-1 g/L. When the OD600 reached over 60, 0.4 mM IPTG was added to induce protein expression. The fed-batch was harvested when the cells reached stationary phase. Finally, the fermentation process was stopped when it reached the stationary phase. The production titer of sdAb-CD after purification was around 160-400 mg/L. The production titer of TC4 may increase to 1-1.3 g/L after process optimization.

### Example 7. Large Scale Purification of the sdAb-CD Fusion Proteins

Fusion Proteins with HIS-Tag

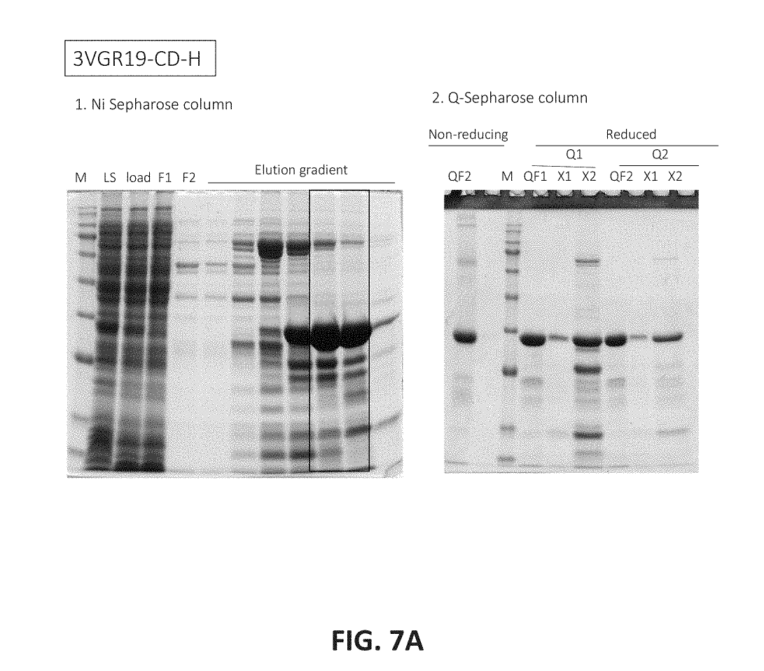

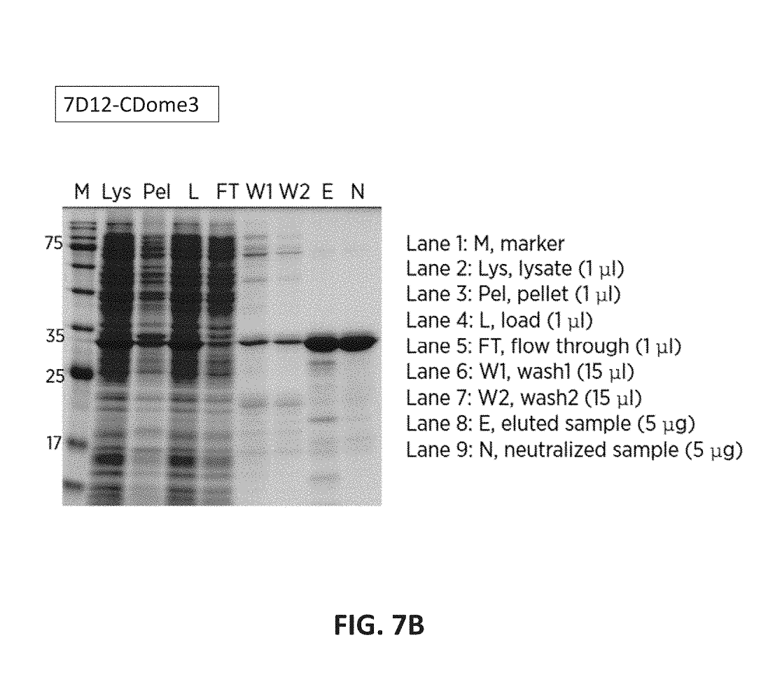

The cell pellet was resuspended with a buffer that is composed of 20 mM TrisHCl, 0.5 M NaCl, 20 mM imidazole, and 5% Glycerol, pH 8.0, and homogenized by homogenizer (APV2000) twice at 850-950 bar for less than 10 minutes. The resulting homogenate was clarified by centrifugation at 22000×g for 60 minutes at 4° C. and filtered. The cell lysate was then applied to an FPLC with a Ni Sepharose column followed by an ion-exchange Q-Sepharose column. The cell lysate was loaded onto the Ni Sepharose column with 20 mM TrisHCl, 0.5 M NaCl, 20 mM imidazole, and 5% Glycerol, pH 8.0, and then eluted with a gradient of 0-500 mM imidazole in 20 mM TrisHCl, 0.5 M NaCl, and 5% Glycerol, pH 8.0. The eluent was loaded onto the ion-exchange Q-Sepharose column with 20 mM TrisHCl, 170 mM NaCl, 5% glycerol, pH 8.0, and the flow through was collected as purified product and then washed with 20 mM TrisHCl, 1000 mM NaCl, 5% glycerol, pH 8.0, to remove impurities and aggregates. The purified fusion proteins were analyzed by SDS-PAGE. The results show that all the sdAb-CD fusion proteins showed high purity after purification (FIG. 7A).

Fusion Proteins without HIS-Tag

The cell pellet was resuspended in a buffer that is composed of 20 mM Tris-HCl and 150 mM NaCl in 5% glycerol at pH 8.0, and homogenized by homogenizer (APV2000) twice at 850-950 bar for less than 10 minutes. The resulting homogenate was clarified by centrifugation at 22000×g for 60 minutes at 4° C. and filtered. The filtrate was purified by rProteinA affinity column (Repligen) followed by an ion-exchange column anion exchanger Q sepharose (GE). The buffer system for rProteinA affinity column comprising 5% glycerol, 20 mM Tris-HCL and 150 mM NaCl at pH 8.0 (for binding and wash), and then was eluted with 50 mM Glycine, 5% glycerol, pH 3.0 buffer, after elution the product was neutralized with 1 M Tris-HCl, pH 9.0 in a ratio of 1 to 25. The buffer for ion-exchange column chromatography comprised 5% glycerol, 20 mM Tris-HCl and 150 mM NaCl at pH 8.0. The purified product was then filtrated by 5 kDa (pore size) TFF system to concentrate the product (Tangential flow filtration) (Merck Millipore), and the buffer was exchanged by 10 kDa Amicon filter (Merck Millipore). (FIG. 7B).

### Example 8. Size-Exclusion Chromatography Analysis of the sdAb-CD Fusion Proteins

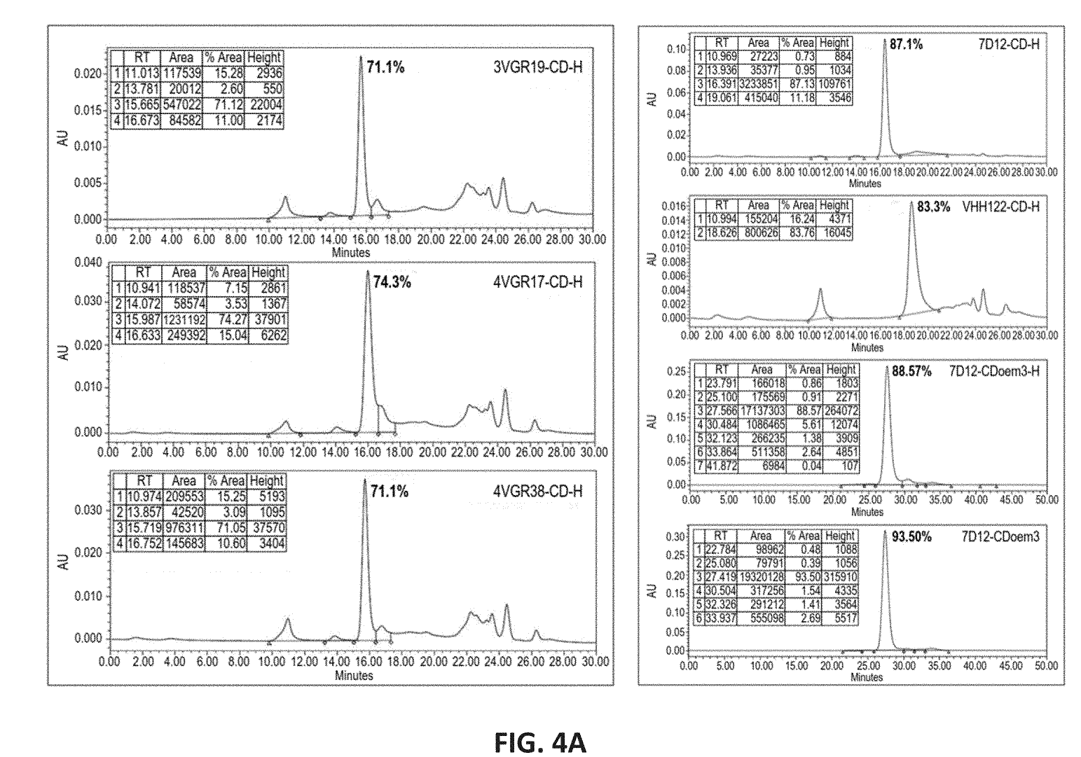

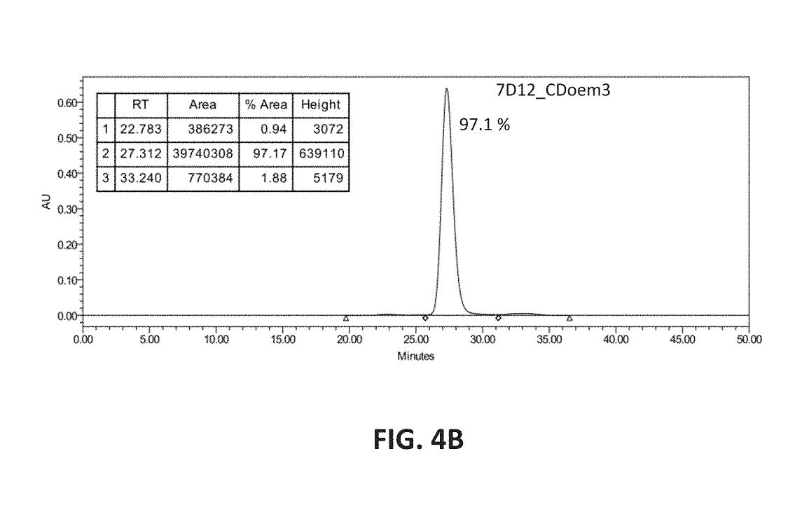

To examine the purity of each fusion protein, SEC-HPLC chromatography using BioSep SEC-s2000 (Phenomenex) or Superdex 200 Increase (GE Healthcare) 10/300 column resin was performed. Samples of the protein produced as above were first diluted to 1 or 2 mg/mL in PBS containing 5% glycerol. The samples were filtered using a 0.2 μm syringe filter (PureTech) and placed in a PP Insert (Thermo) for SEC-HPLC analysis. The mobile phase for BioSep SEC-s2000 was 0.1M sodium phosphate with 0.3 M sodium chloride, pH 7.0. The mobile phase for Superdex 200 Increase was 5% glycerol in PBS. The flow rate for either column was 0.5 mL/min. The column temperature was set at 25±2° C. and the auto-sampler temperature was set at 10±2° C. Proteins were detected by absorbance at UV280. The results showed that the purity for anti-VEGFR2 sdAb-CD fusion proteins 3VGR19-CD-H, 4VGR17-CD-H, and 4VGR38-CD-H was about 71%, about 74%, and about 71% respectively (FIG. 4A). The purity for anti-EGFR sdAb-CD fusion proteins VHH122-CD-H and 7D12-CD-H was about 83% and about 87%, respectively (FIG. 4A). For 7D12-CDoem3-H and 7D12-CDoem3, the purity was about 88% and about 93%, respectively (FIG. 4A). After optimization of the purification process, the purity may reach about or over 95% (FIG. 4B).

### Example 9. Cytosine Deaminase Activity of the sdAb-CD Fusion Proteins

To examine the cytosine deaminase activity of the CD fusion proteins, sdAb-CD fusion proteins were prepared as above, serial diluents of the fusion protein samples were mixed with 20 mM 5-FC in buffer comprising 0.25% BSA and 0.05% Tween 20, and the mixtures were incubated at 37° C. for 90 minutes. The reactions were then stopped with 10% trichloroacetic acid and the mixtures were centrifuged at 4° C. for supernatant collection. The presence of 5-FC and 5-FU was detected by absorbance at 290 nm and 255 nm, respectively.

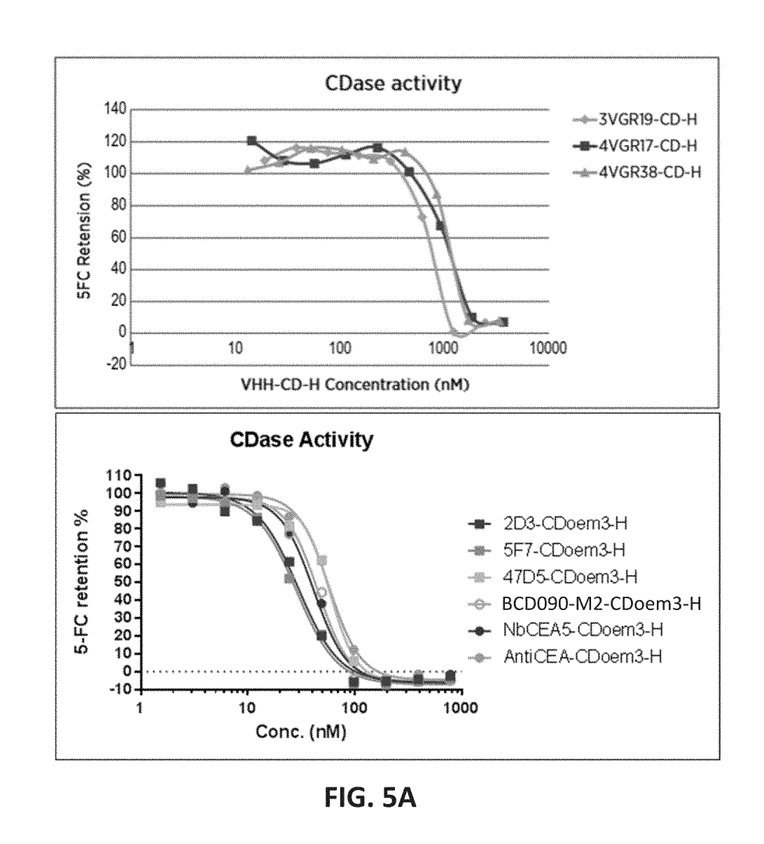

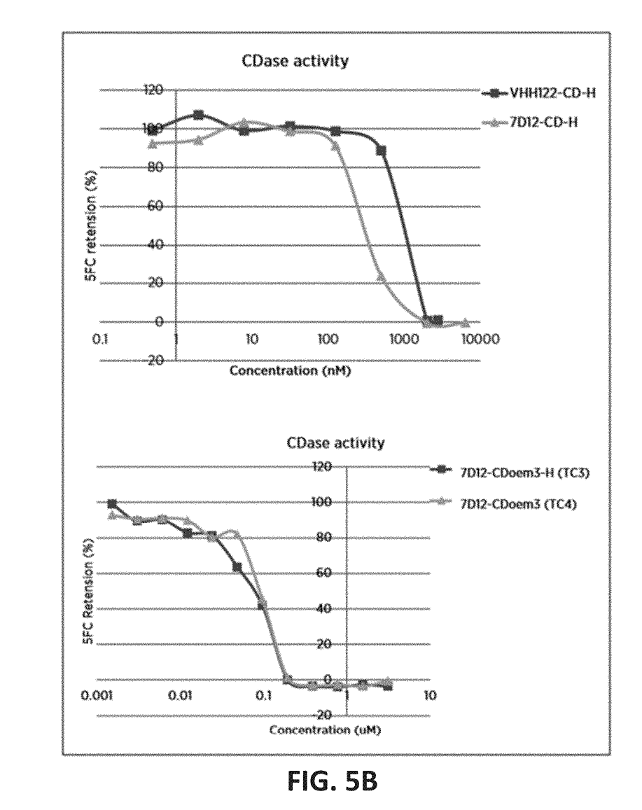

The results demonstrate that the tested sdAb-CD fusion proteins targeting VEGFR2 converted 5-FC to 5-FU (FIG. 5A). Similar results were obtained for sdAb-CD fusion proteins targeting EGFR, HER2, HER3, or CEA (FIG. 5A and FIG. 5B).

### Example 10. Antigen Binding Affinity of sdAb-CD Fusion Proteins

The binding between human VEGFR2 and the sdAb-CD fusion proteins disclosed herein was tested by ELISA. For the VEGFR2 binding assay, the coating antibody Human VEGFR2-Fc (Sino Biological) was diluted to 1 μg/ml in coating buffer (100 mM NaHCO₃+32 mM Na₂HCO₃). Blocking was performed by incubating with blocking buffer (0.25% BSA, 0.05% Tween-20, 0.05% NaN₃, 1 mM EDTA) for 2 hours. The tested fusion protein samples were prepared in blocking buffer and added to wells for binding. Rabbit anti-His-HRP (abcam) was diluted 5000-fold in a buffer comprising 0.25% BSA and 0.05% Tween 20 for detection.

For the EGFR binding assay, ELISA plates were coated with hEGFR-Fc (Sino Biological) with concentration 1 μg/mL in coating buffer. Blocking was performed by incubating with blocking buffer (0.25% BSA, 0.05% Tween-20, 0.05% NaN₃, 1 mM EDTA) for 2 hours. After blocking, the samples were serial diluted in blocking buffer and added to wells for binding 1 hour. Finally, the secondary antibodies (Rabbit anti-HIS-HRP) were diluted in buffer comprising 0.25% BSA and 0.05% Tween 20 for detection.

For the HER2, HER3 and CEA binding assays, the coating antibody for each assay was human ErbB2/Her2-Fc (Acro biosystem), human ErbB3/Her3-Fc (Acro biosystem), and human CEACAM5-Fc (novoprotein), respectively.

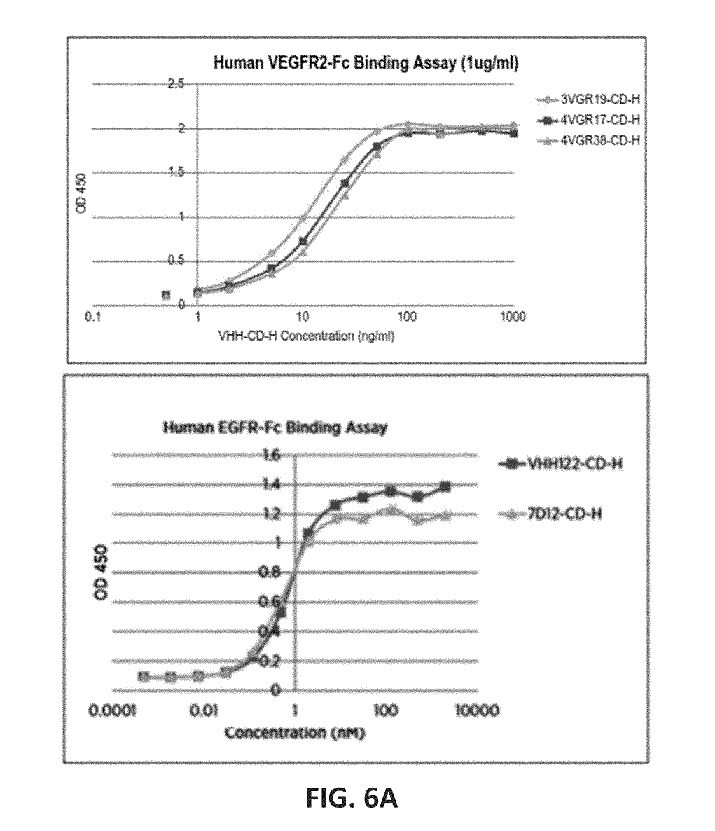

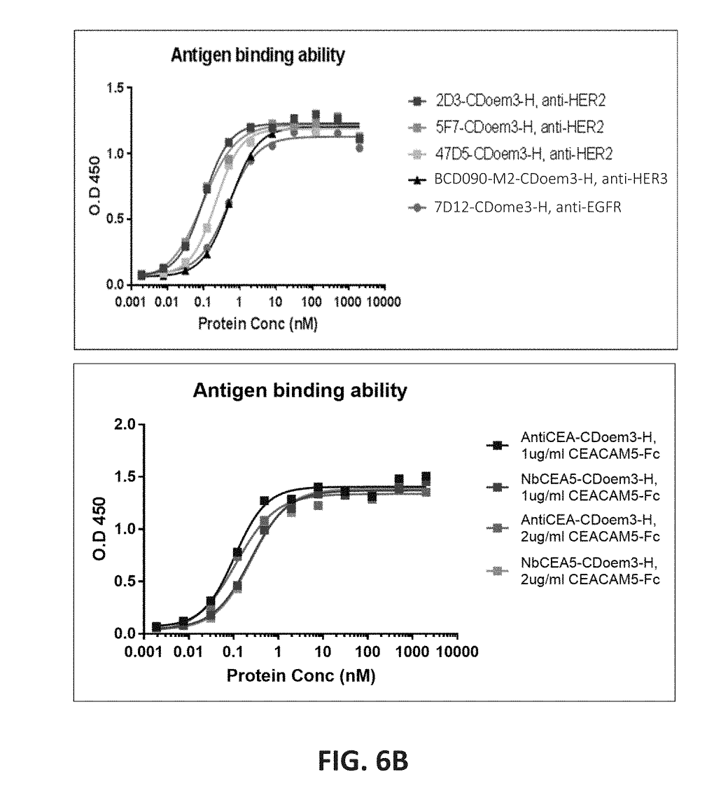

The results showed that 3VGR19-CD-H, 4-VGR17-CD-H, and 4VGR38-CD-H bind to VEGFR2; VHH122CD-H and 7D12-CD-H bind to EGFR; 5F7-CDoem3-H, 47D5-CDoem3-H, and 2D3-CDoem3-H bind to HER2, NbCEA5-CDoem3-H and AntiCEA-CDoem3-H bind to CEACAM5; and BCD090-M2-CDoem3-H binds to HER3 (FIG. 6A and FIG. 6B).

### Example 11. Cell-Based Cytotoxic Assay for sdAb-CD Fusion Proteins

Protocol A:

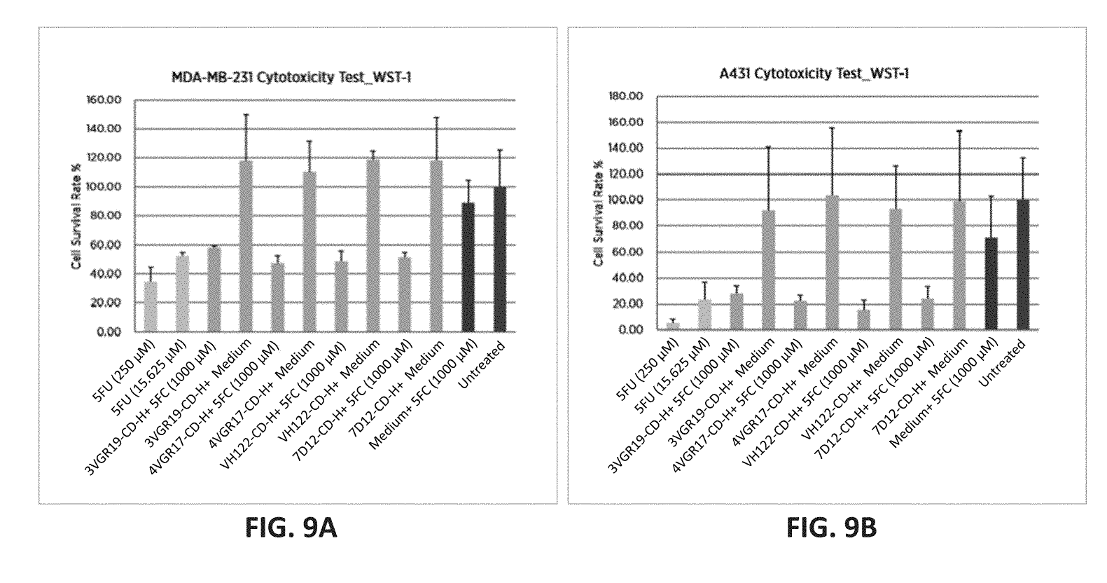

The cytotoxicity of several anti-EGFR sdAb-CD fusion proteins combined with 5-FC was tested on MDA-MB-231 (human breast carcinoma) and A431 (human epidermoid carcinoma) EGFR-expressing cancer cell lines. The cells were first seeded at 30,000 cells/well in 96-well plates and incubated at 37° C. overnight in growth medium (DMEM (Gibco) plus 10% FBS). After 16-18 hours of incubation, the wells were washed once with PBS. One hundred microliters of fusion protein at 100 μg/ml were added to each well and incubated for 1 hour at 37° C. After washing with PBS to remove excess fusion protein, 100 μl of the 5-FC or 5-FU at indicated concentrations were added to the respective wells for 72 hours incubation. Lastly, 10 μl/well of cell proliferation reagent WST-1 (Roche) were added and the cells incubated at 37° C. for 4 hours. Cell survival was measured using an ELISA reader at absorbance OD450 (WST-1) and OD690 (reference wavelength). The results demonstrate that the combination of each tested sdAb-CD fusion protein with 5-FC decreased the cancer cell survival in both MDA-MB-231 and A431 cell lines (FIG. 9A and FIG. 9B).

Protocol B:

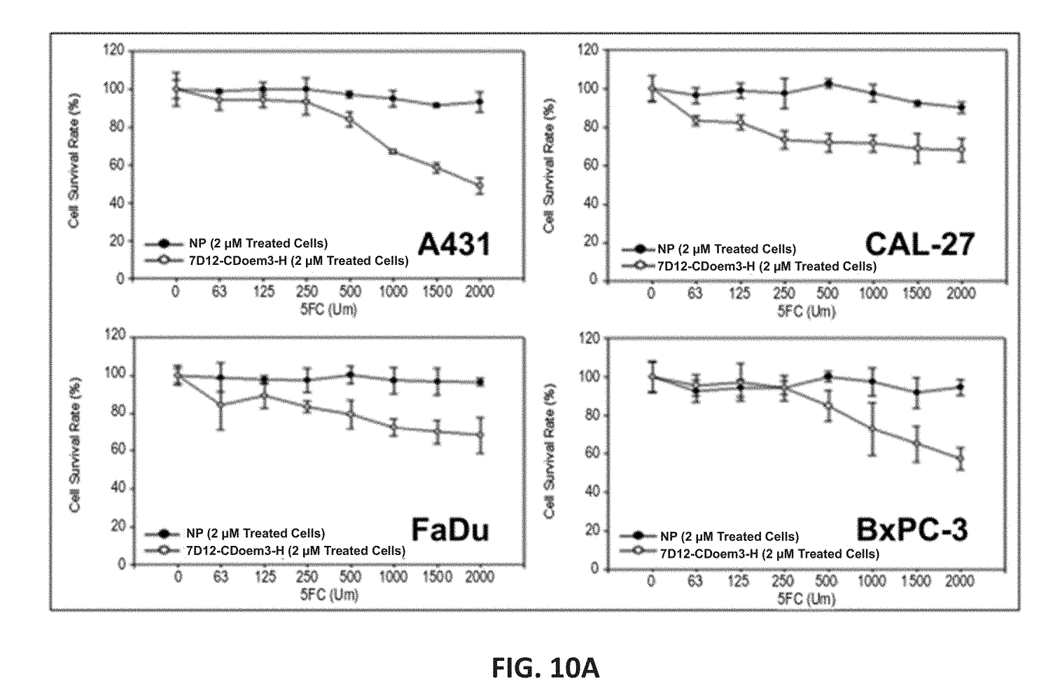

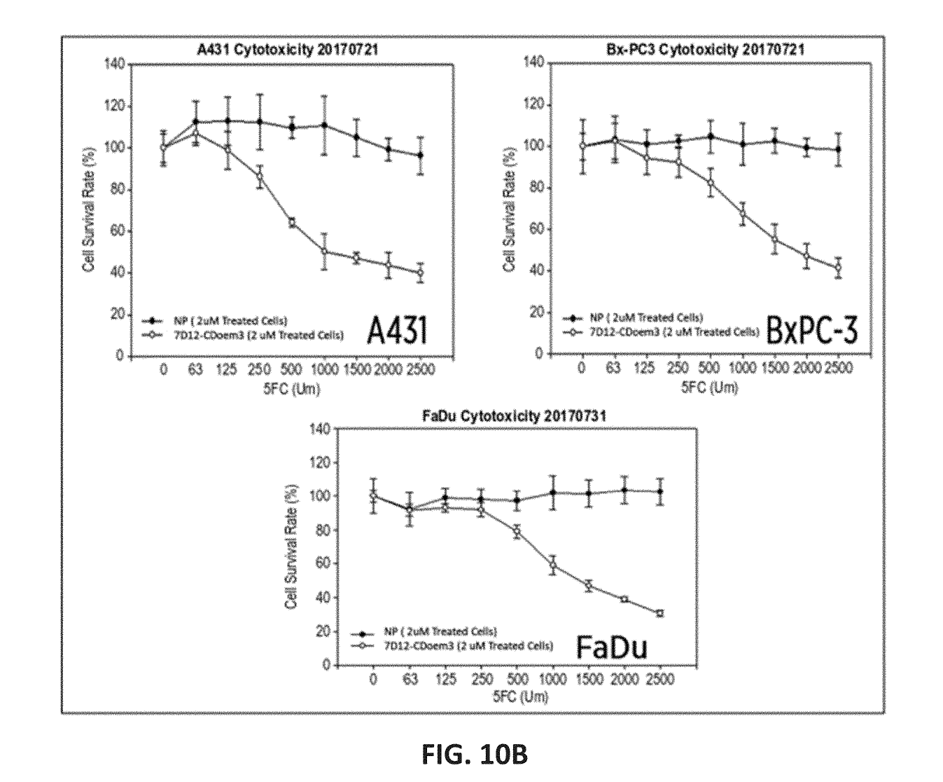

In alternative assays, the cytotoxicity of anti-EGFR sdAb-CD fusion proteins of the disclosure combined with 5-FC was tested on A431, Bx-PC3, Cal-27, and FaDu cancer cell lines. A CD protein with a C-terminal His-tag (CDoem3-H) was used as negative protein control (NP). First, the cells were re-suspended with 4 mL culture medium containing 2 μM NP or sdAb-CD and incubated in 37° C. for 1 hour. Each cell line used the growth medium suggested by ATCC: A431: CRL-1555™; FaDu: HTB-43™; Cal 27: CRL-2095™; BxPC-3:CRL-1687™. The cells were then washed three times with PBS and re-seeded at 30,000 cells/well in a 96-well plate. 5-FC at the indicated concentrations was added to the wells and incubated at 37° C. for 72 hours. After incubation, 10 μl of cell proliferation reagent WST-1 were added and the mixtures were incubated at 37° C. for 3 hours. Cell survival was measured using an ELISA reader at OD450 and OD690. The results indicate that anti-EGFR sdAb-CD fusion proteins decreased the survival rate for all tested cancer cell lines (FIG. 10A and FIG. 10B).

### Example 12. Test of sdAb-CD Proteins on A431 Xenograft Model

The therapeutic effects of 7D12-CDoem3 or 7D12-CDoem3-H fusion proteins were evaluated with an A431 xenograft model in NOD-SCID male mice (LASCo). 2.5×10⁶ A431 tumor cells were injected subcutaneously into the right flank of mice weighed between 20-27 g. After the tumors' size reached 200-300 mm³ (approximately 10 days after tumor transplantation), the mice were randomly assigned to different treatment groups (n=6). The indicated amounts of vehicle (PBS) 5-FU (intraperitoneally), 5-FC (intraperitoneally), and sdAb-CD fusion proteins (intravenously) were administered twice per week for 4 weeks. The solid mass of the tumors was measured and the significance of difference in tumor volume was evaluated by Student's t-test.

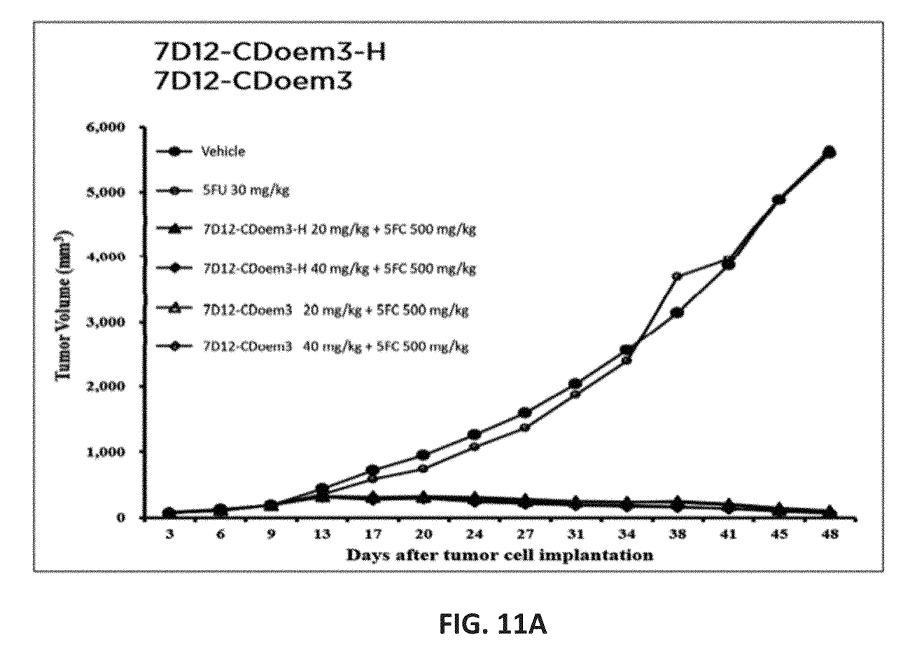

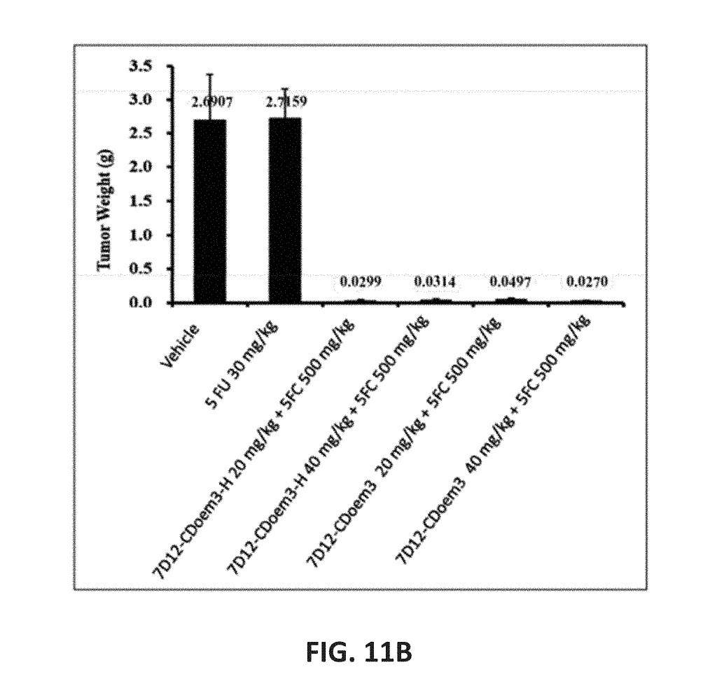

Intravenous administration of 7D12-CDoem3-H or 7D12-CDoem3 at either 20 mg/kg or 40 mg/kg with intraperitoneal injection of 5-FC at a dose of 500 mg/kg resulted in a significant reduction in the growth of A431 tumors, as shown by the reductions in both tumor volume and tumor weight compared to the vehicle-treated group after tumor cell transplantation (p<0.01) (FIG. 11A and FIG. 11B).

This confirms that co-administration of 7D12-CDoem3-H or 7D12-CDoem3 with 5-FC can have an inhibitory effect on A431 cancer cell growth in vivo.

### Example 13. T Cell Epitope Mapping

EpiScreen™ T cell epitope mapping of 91 peptides resulted in positive T cell responses against six peptides comprising six epitopes. Using iTope™, nine potential HLA-DR restricted binding sequences were identified in the peptides that induced positive T cell proliferation responses.

In summary, six T cell epitopes were identified within the 7D12-CDoem3 sequence, epitopes 1-3 located at the framework region of 7D12 sdAb, and epitope 4-6 located at the CD region.

| Epitope | HLA-DR restricted epitope | SEQ ID NO |
|---|---|---|
| Epitope 1 | SVQTGGSLRL | 63 |
| Epitope 2 | LKPEDTAIY | 64 |
| Epitope 3 | IYYCAAAAGS | 65 |
| Epitope 4 | YTTLSPCDM | 66 |
| Epitope 5 | MCTGAIIMY | 67 |
| Epitope 6 | VVVVDDERCKK | 68 |

### Example 14. Single Epitope Variant of sdAb-CD Fusion Proteins

Individual single epitope variants of 7D12-CDoem3-H were designed to evaluate whether these variants can eliminate immunogenicity while retaining its structure, solubility, CDase activity, and antigen-binding activity.

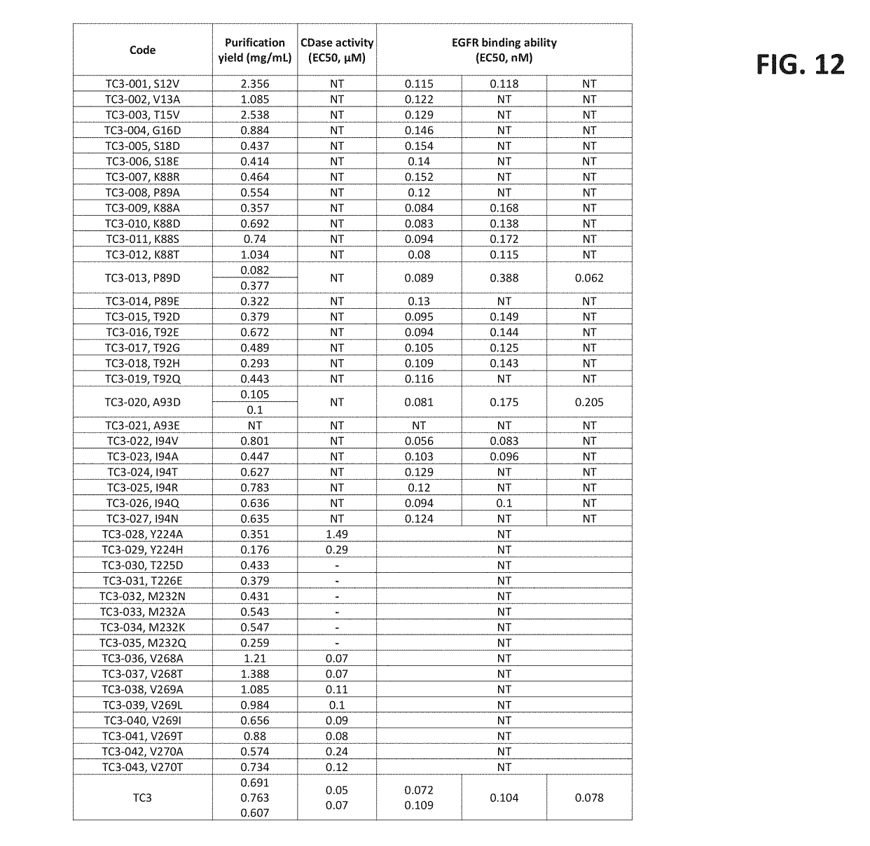

Expression plasmids of 43 single epitope variants were generated by overlapping PCR. These fusion protein variants were generated and the CD activity, EGFR-binding activity and expression profile thereof accessed. The results are summarized in FIG. 12.

The data indicated that all of the EGFR-binding domain substitutions (TC3-001 TC3-027) had no obvious effect on EGFR-binding ability. All the eight variants with modified epitope 4 and epitope 5 (TC3-028 TC3-035) lost their CDase activity. The variants with modified epitope 6 (TC3-036˜TC3-043) were less affected.

Based on the expression titer, biological activity, and the sequence similarity to human germ line (EGFR-binding domain), the following variants were selected for multi-mutation design (numbering relative to SEQ ID NO: 17):

a. Epitope 1: S12V, V13A, T15V, G16D, and S18D

### b, Epitope 2: K88R, P89A, I94V, K88D, K88T

c. Epitope 3: I94R, I94Q
d. Epitope 4: Y224H
e. Epitope 6: V268A, V268T, V269T

### Example 15. Single Epitope Variant of sdAb-CD Fusion Proteins

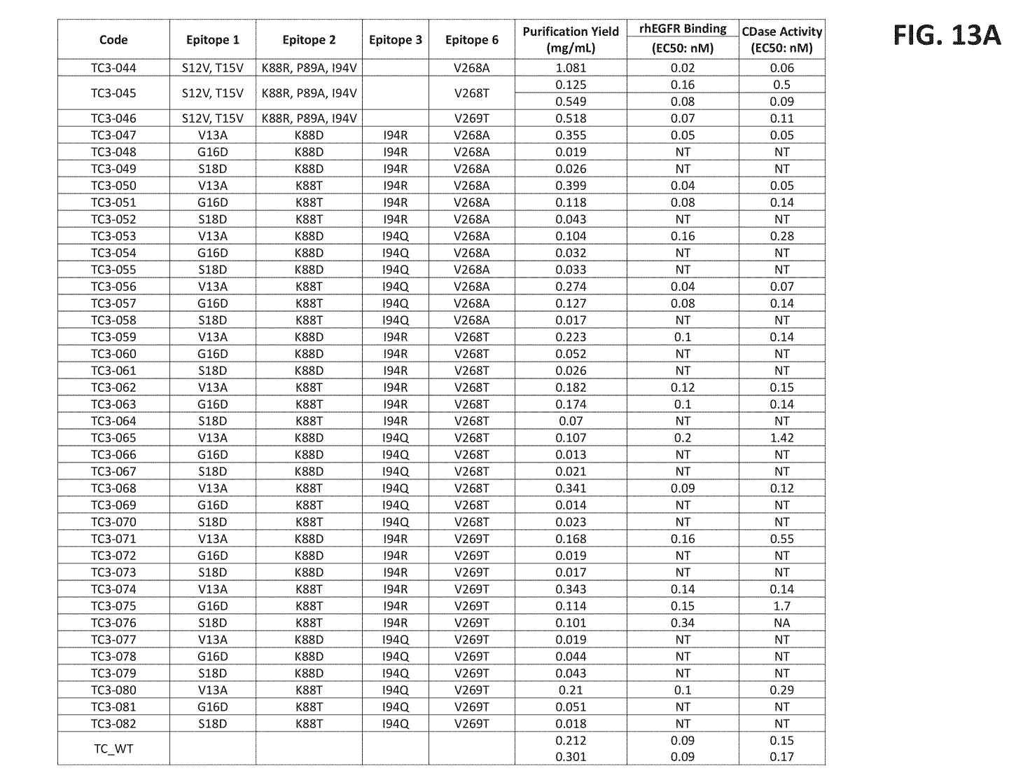

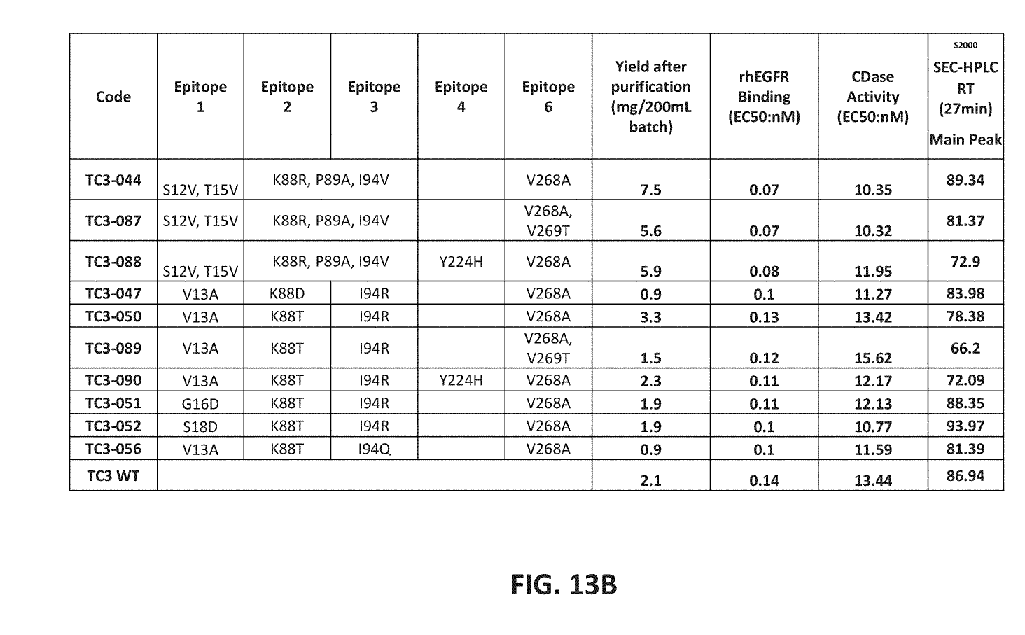

Expression plasmids of 43 multi-mutation variants of TC3 were generated by overlapping PCR. These fusion protein variants were generated and the CDase activity, EGFR-binding activity and expression profile thereof were accessed. The results are summarized in FIG. 13A and FIG. 13B.

Based on the expression titer, biological activity, phycological characteristics, and the coverage to eliminate T cell epitopes, five candidate multi-variants were further selected to assess their immunogenic potential.

### Example 16. Immunogenicity Analysis

Five variants of 7D12-CDoem3 were assessed for their immunogenic potential using EpiScreen™ time course T cell assays. Bulk cultures were established using CD8+-depleted PBMC, and CD4+ T cell proliferation was measured at various time points after the addition of the samples by incorporation of [3H]-Thymidine. IL-2 secretion was also measured by ELISpot after eight days. TC4 (sample 1) and other 4 fusion protein candidates were tested.

| Sample | Fusion protein Name | SEQ ID NO |
|---|---|---|
| Sample 1 | TC4-WT (7D12-CDoem3) | 19 |
| Sample 2 | TC4_44 (S12V, T15V, K88R, | 182 |
| | P89A, I94V, V268A) | |
| Sample 3 | TC4_50 (V13A, K88T, I94R, | 183 |
| | V268A) | |
| Sample 4 | TC4_51 (G16D, K88T, I94R, | 184 |
| | V268A) | |
| Sample 5 | TC4_87 (S12V, T15V, K88R, | 185 |
| | P89A, I94V, V268A, V269T) | |

EpiScreen™ Time Course T Cell Proliferation Assays

A cohort of 52 donors was selected to best represent the number and frequency of HLA-DR and HLA-DQ allotypes expressed in European/North American and the world population. PBMCs from each donor were resuspended in AIM-V® to 4-6×10⁶ PBMC/mL. The final sample concentration of the tested recombinant protein was 0.3 μM. Cultures were incubated for a total of 8 days. On days 5, 6, 7, and 8, the cells in each well were pulsed with 0.75 μCi [3H]-Thymidine (Perkin Elmer®, Beaconsfield, UK) in 100 μL AIM-V® culture medium and incubated for a further 18 hours before harvesting onto filter mats (Perkin Elmer®, Beaconsfield, UK) using a TomTec Mach III cell harvester. Counts per minute (cpm) for each well were determined by scintillation counting on a 1450 Microbeta Wallac Trilux Liquid Scintillation Counter (Perkin Elmer®, Beaconsfield, UK) in paralux, low background counting.

EpiScreen™ IL-2 ELISpot Assays

PBMCs from the same cohort of donors were used for the IL-2 ELISpot assay. ELISpot plates (Millipore, Watford, UK) were pre-wetted and coated overnight with IL-2 capture antibody (R&D Systems, Abingdon, UK). The cell density for each donor was adjusted to 4-6×10⁶ PBMC/ml in AIM-V® culture medium and 100 μL of cells were added to each well. Fifty microliters of samples and controls were added to the appropriate wells. After an 8-day incubation period, ELISpot plates were developed according to the manufacturer's instructions (R&D Systems). Briefly, the plates were washed prior to the addition of biotinylated detection antibody (R&D Systems, Abingdon, UK). Following incubation at 37° C. for 1.5 hours, plates were further washed in PBS (×3) and filtered streptavidin-AP (R&D Systems, Abingdon, UK) was added for 1 hour (incubation at room temperature). Streptavidin-AP was discarded, and plates were washed in PBS (×3). One hundred microliters BCIP/NBT substrate (R&D Systems, Abingdon, UK) were added to each well and incubated for 30 minutes at room temperature. Spot development was stopped by washing the wells and the backs of the wells three times with dH2O. Dried plates were scanned on an Immunoscan® Analyser and spots per well (spw) were determined using Immunoscan® Version 5 software.

For proliferation assays and IL-2 ELISpot assays, an empirical threshold of a SI equal to or greater than 1.9 (SI≥1.90) has been previously established whereby samples inducing responses above this threshold are deemed positive. For proliferation (n=3 per time point) and ELISpot (n=6), positive responses were defined by statistical and empirical thresholds as follows:

1. Significance (p<0.05) of the response by comparing cpm or spw of test wells against medium control wells using unpaired two sample Student's t-test.

2. SI≥1.90, where SI=mean of test wells (cpm or spw)/baseline (cpm or spw).

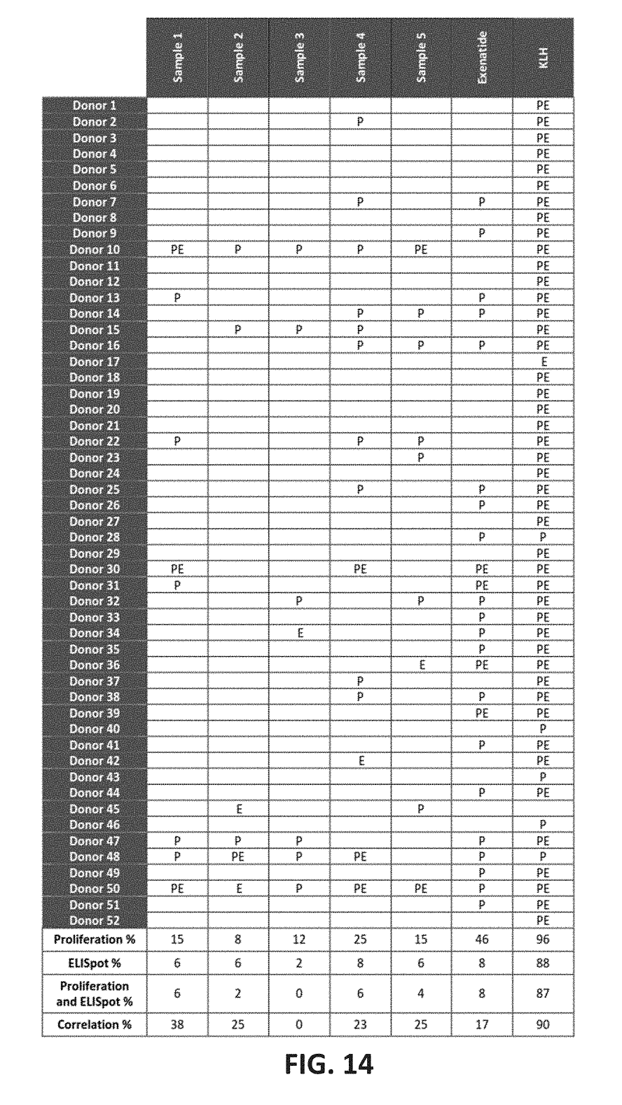

FIG. 14 shows the summary of healthy donor T cell proliferation and IL-2 ELISpot responses. Positive T cell responses for proliferation (SI≥1.90, significant p<0.05) during the entire time course days 5-8 (“P”), and IL-2 (SI≥1.90, significant p<0.05) ELISpot (“E”) are indicated. The frequency of positive responses for proliferation and IL-2 ELISpot assays are shown as a percentage at the bottom of the columns. Correlation is expressed as the percentage of proliferation responses also positive in the ELISpot assay.

Due to the low frequency of positive responses in the IL-2 ELISpot assay, ranking the samples base on proliferation responses only. A high frequency of positive responses (SI≥1.90,p<0.05) was induced by sample 4 with 25% of the donor cohort responding. Samples 1, 3, and 5 induced positive responses in between 12% and 15% of the donor cohort and 8% of donors responded positively to sample 2. Sample 2 yielded the lowest risk of clinical immunogenicity.

### Example 17. Functional Analysis of De-Immunized sdAb-CD

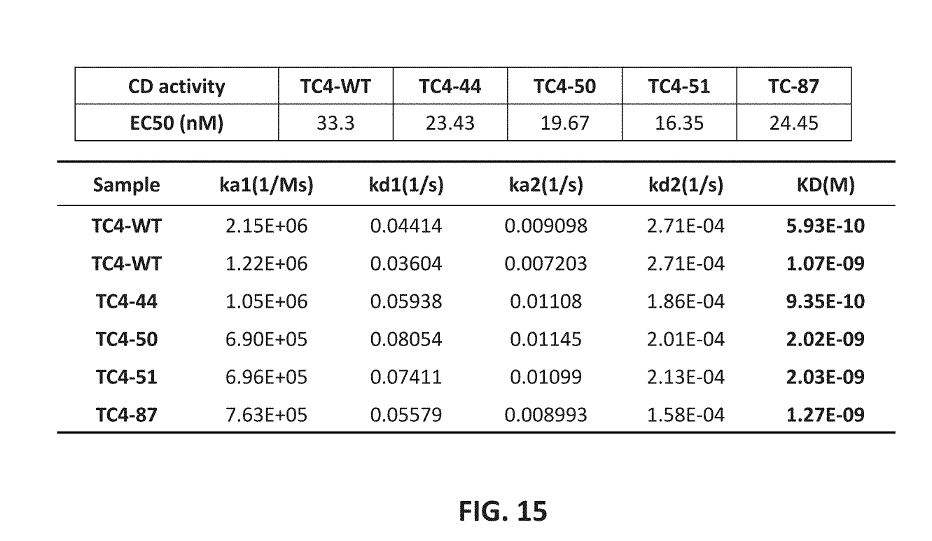

The CD activity and EGFR-binding activity of de-immunized TC4 variants were analyzed and are shown in FIG. 15.

CD activity was assessed by the method described in Example 8.

Binding affinity between TC4 related proteins and EGFR-Fc were measured by Surface Plasmon Resonance (SPR). SPR assay was performed by Biacore T100 (GE Healthcare). Anti-human IgG(Fc) antibody at 25 μg/mL was diluted in immobilization buffer (10 mM NaOAc, pH 5.0). Antibody was immobilized on a CMS chip (Series S Sensor Chip CMS, GE; Cat. No.:29104988) via standard amine coupling chemistry according to manufacture protocol. The procedure should result in immobilization level of ˜9000 RU. The mobile phase was PBST and the temperature in the flow cells was maintained at 25° C. After immobilization, the ligand solution (humans EGFR-Fc protein, 2 μg/mL in mobile phase) was injected into system with contact time 120 s and flow rate 10 μL/min. Next, TC4-wt or TC4-mutants, which were diluted to 0.74, 2.22, 6.67, 20, and 60 nM in PBST, was injected to systems from low concentration to high concentration. The condition for analyte injection was contact time 120 seconds for each concentration and dissociate samples for 600 seconds at the final step. The analysis was performed with Biacore T100 Evaluation Software. The analytical result of single-cycle kinetics analyte for each sample was fit by two state reaction to determine the K_{D} value. The result criterion was that the maximum response (Rₘₐₓ) should be in the range of 50˜250 RU.

Data suggested that the EGFR-binding ability and CD activity of TC4 variants TC4-44, 50, 51, and 87 were similar compared to wild type TC4 (FIG. 15).

What we claim is:

1. A fusion protein comprising formula I or formula II, wherein:
N-(L)n-C; and formula I is
C-(L)n-N; formula II is;
wherein N is a single-domain antibody (sdAb) or a functional variant thereof, L is a peptide linker, n=0-50, and C is a cytosine deaminase (CD) protein or a functional variant thereof.

2. The fusion protein of claim 1, wherein n=0, 1, or 2.

3. The fusion protein of claim 1, wherein the sdAb or functional variant thereof binds to a target.

4. The fusion protein of claim 3, wherein the target is selected from the group consisting of EGFR, 5T4, A33, AFP, Beta-catenin, BRCA1, BRCA2, C242, CCR4, CD152, CD19, CD20, CD200, CD22, CD221, CD23, CD30, CD3, CD37, CD40, CD44, CD5, CD51, CD52, CD56, CD64, CD74, CD80, CDCP1, c-KIT, COX-2, cMET, CSF1R, CTLA-4, ErbB2, ErbB3, EGF2, FGFR1, FGFR2, FGFR3, FLT3, HER2, HER3, HIF-Ia, HLA-DR, IGF-IR, mTOR, NPC-1C, P53, PDGFRα, PDGFRβ, PLGF, PSA, RGMa, RoN, TNF, TP53, TPD52, VEGFR1, VEGFR2, VEGFR3, CA-IX, αvβ3, α5β1, FAP, glycoprotein 75, TAG-72, MUC16, NR-LU-13, SLAMF7, EGP40, BAFF, PRL-3, carcinoembryonic antigen (CEA), prostate-specific membrane antigen, MART-1, gp100, Cancer-testis (CT) antigens (e.g. NY-ESO-1, MAGE-A3, MAGE-A1), hTERT, MCC, Mum-1, ERBB2IP, EpCAM, TfR, integrin α6β4, HGFR, PTP-LAR, CD147, CDCP1, CEACAM6, JAM1, integrin α3β1, integrin αvβ3, PD-L1, AXL, CDH6, DLL3, EDNRB, EFNA4, NEPP3, EPHA2, FOLR1, LewisY, GPNMB, GUCY2C, HAVCR1, Integrin α, LYPD3, Mesothelin, MUC1, NECTIN4, NOTCH3, PTK7, SLC34A2, SLC39A6, SLC44A4, SLITRK6, STEAP1, TACSTD2, TPBG, TIM-1, GD2, and nicotinic acetylcholine receptor (nAChR).

5. The fusion protein of claim 1, wherein the sdAb or functional variant thereof comprises:
(a) a complementarity determining region 1 (CDR1) selected from the group consisting of SEQ ID NOs: 28, 31, and 34; a CDR2 selected from the group consisting of SEQ ID NOs: 29, 32, and 35; and a CDR3 selected from the group consisting of SEQ ID NOs: 30, 33, and 36; or
(b) a CDR1 selected from the group consisting of SEQ ID NOs: 37 and 40, a CDR2 selected from the group consisting of SEQ ID NOs: 38 and 41, and a CDR3 selected from the group consisting of SEQ ID NOs: 39 and 42; or
(c) a CDR1 selected from the group consisting of SEQ ID NOs: 199, 202 and 205; a CDR2 selected from the group consisting of SEQ ID NOs: 200, 203 and 206; and a CDR3 selected from the group consisting of SEQ ID NOs: 201, 204 and 207; or
(d) a CDR1 selected from the group consisting of SEQ ID NOs: 208, a CDR2 selected from the group consisting of SEQ ID NOs: 209, and a CDR3 selected from the group consisting of SEQ ID NOs: 210; or
(e) a CDR1 selected from the group consisting of SEQ ID NOs: 211 and 214, a CDR2 selected from the group consisting of SEQ ID NOs: 212 and 215, and a CDR3 selected from the group consisting of SEQ ID NOs: 213 and 216.

6. The fusion protein of claim 5, wherein the sdAb or functional variant thereof comprises the amino acid sequence of SEQ ID NO: 23 (3VGR19), SEQ ID NO: 24 (4VGR17), SEQ ID NO: 25 (4VGR38), SEQ ID NO: 26 (VHH122), SEQ ID NO: 27 (7D12), SEQ ID NO: 69 (2D3), SEQ ID NO: 70 (5F7), SEQ ID NO: 71 (47D5), SEQ ID NO: 75 (BCD090-M2), SEQ ID NO: 77 (ABS29544.1), or SEQ ID NO: 79 (NbCEA5).

7. The fusion protein of claim 1, wherein at least one peptide linker comprises the amino acid sequence of SEQ ID NO: 1, SEQ ID NO: 4, SEQ ID NO: 5, or SEQ ID NO: 188.

8. The fusion protein of claim 1, wherein the CD protein or functional variant thereof is (i) a bacterial CD protein or a functional variant thereof or (ii) a yeast CD or a functional variant thereof.

9. The fusion protein of claim 8, wherein the CD protein or functional variant thereof comprises an amino acid sequence that is at least 90% identical, at least 91% identical, at least 92% identical, at least 93% identical, at least 94% identical, at least 95% identical, at least 96% identical, at least 97% identical, at least 98% identical, at least 99% identical, or 100% identical to an amino acid sequence selected from the group consisting of SEQ ID NOs: 21, 22, 186, and 187.

10. The fusion protein of claim 9, wherein the CD protein or functional variant thereof is a functional variant of a starting amino acid sequence selected from the group consisting of SEQ ID NOs: 21, 22, 186, and 187; wherein
(a) when the starting amino acid sequence is SEQ ID NO: 21 or SEQ ID NO: 22, the functional variant comprises at least one mutation selected from the group consisting of Y84A, Y84H, T85D, T86E, M92N, M92A, M92K, M92Q, V128A, V128T, V129A, V129L, V129I, V129T, V130A, and V130T; and
(b) when the starting amino acid sequence is SEQ ID NO: 186 or 187, the functional variant comprises at least one mutation selected from the group consisting of Y85A, Y85H, T86D, T87E, M93N, M93A, M93K, M93Q, V129A, V129T, V130A, V130L, V130I, V130T, V131A, and V131T.

11. The fusion protein of claim 1, wherein the fusion protein comprises an amino acid sequence selected from the group consisting of:
(i) SEQ ID NO: 7,
(ii) amino acids 1-297 of SEQ ID NO: 7,
(iii) SEQ ID NO: 9,
(iv) amino acids 1-297 of SEQ ID NO: 9,
(v) SEQ ID NO: 11,
(vi) amino acids 1-297 of SEQ ID NO: 11,
(vii) SEQ ID NO: 13,
(viii) amino acids 1-297 of SEQ ID NO: 13,
(ix) SEQ ID NO: 15,
(x) amino acids 1-297 of SEQ ID NO: 15,
(xi) SEQ ID NO: 17, and
(xii) SEQ ID NO: 19.

12. The fusion protein of claim 1, wherein the fusion protein further comprises at least one de-immunizing mutation in at least one T cell epitope, wherein the at least one T cell epitope is selected from the group consisting of Epitope 1 (SEQ ID NO:
63), Epitope 2 (SEQ ID NO: 64), Epitope 3 (SEQ ID NO: 65), Epitope 4 (SEQ ID NO: 66), Epitope 5 (SEQ ID NO: 67), and Epitope 6 (SEQ ID NO: 68).

13. The fusion protein of claim 1, wherein the fusion protein consists essentially of the amino acid sequence of SEQ ID NO: 17, 19, 93-185, or amino acids 1-297 of any one of SEQ ID NOs: 93-181.

14. The fusion protein of claim 13, wherein the fusion protein consists essentially of an amino acid sequence selected from the group consisting of SEQ ID NOs: 19, 182, 183, 184, and 185.

15. A pharmaceutical composition comprising an effective amount of at least one fusion protein of claim 1 and at least one pharmaceutically acceptable carrier or excipient.

16. A method of treating cancer in a subject in need thereof, the method comprising administering to the subject an effective amount of at least one fusion protein of claim 1.

17. A nucleic acid molecule comprising a nucleic acid sequence encoding a fusion protein of claim 1.

18. A vector comprising the nucleic acid molecule of claim 17.

19. A host cell comprising the vector of claim 18.

20. A method of making a fusion protein comprising expressing the nucleic acid of claim 17 in a host cell.
